(19) United States
(12) Patent Application Publication
(10) Pub. No.: US 2015/0141438 A1
(43) Pub. Date: May 21, 2015

(54) METHODS FOR DELAYING OR PREVENTING THE ONSET OF TYPE 1 DIABETES

(71) Applicant: Pharmacyclics, Inc., Sunnyvale, CA (US)

(72) Inventor: Peggy L. KENDALL, Nashville, TN (US)

(21) Appl. No.: 14/542,458

(22) Filed: Nov. 14, 2014

Related U.S. Application Data
(60) Provisional application No. 61/905,081, filed on Nov. 15, 2013.

(51) Int. Cl. A61K 31/519

(52) U.S. Cl. CPC A61K 31/519
USPC 514/262.1

(57) ABSTRACT

Described herein are methods for preventing or delaying the onset of Type 1 Diabetes, or inhibiting the maturation of anti-insulin B cells, in an individual in need thereof. The methods include administering to an individual in need thereof ibrutinib, alone or in combination with other Type 1 Diabetes treatments.

### CROSS-REFERENCE

This application claims the benefit of U.S. provisional patent application No. 61/905,081, filed on Nov. 15, 2013, which is herein incorporated by reference in its entirety.

### SUMMARY OF THE INVENTION

Disclosed herein, in certain embodiments, are methods for preventing or delaying the onset of type 1 diabetes in an individual in need thereof comprising administering to the individual in need thereof a composition comprising a therapeutically-effective amount of a compound of Formula A:

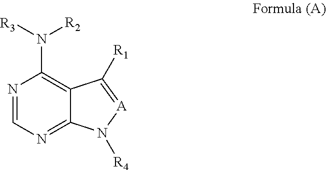

wherein
A is independently selected from N or CR₅;
R₁ is H, L₂-(substituted or unsubstituted alkyl), L₂-(substituted or unsubstituted cycloalkyl), L₂-(substituted or unsubstituted alkenyl), L₂-(substituted or unsubstituted cycloalkenyl), L₂-(substituted or unsubstituted heterocycle), L₂-(substituted or unsubstituted heteroaryl), or L₂-(substituted or unsubstituted aryl), where L₂ is a bond, O, S, —S(═O), —S(═O)₂, C(═O), -(substituted or unsubstituted C₁-C₆ alkyl), or -(substituted or unsubstituted C₂-C₆ alkenyl);
R₂ and R₃ are independently selected from H, lower alkyl and substituted lower alkyl;
R₄ is L₃-X-L₄-G, wherein,
   L₃ is optional, and when present is a bond, optionally substituted or unsubstituted alkyl, optionally substituted or unsubstituted cycloalkyl, optionally substituted or unsubstituted alkenyl, optionally substituted or unsubstituted alkynyl;
   X is optional, and when present is a bond, O, —C(═O), S, —S(═O), —S(═O)₂, —NH, —NR₉, —NHC(O), —C(O)NH, —NR₉C(O), —C(O)NR₉, —S(═O)₂NH, —NHS(═O)₂, —S(═O)₂NR₉—, —NR₉S(═O)₂, —OC(O)NH—, —NHC(O)O—, —OC(O)NR₉—, —NR₉C(O)O—, —CH═NO—, —ON═CH—, —NR₁₀C(O)NR₁₀—, heteroaryl, aryl, —NR₁₀C(═NR₁₁)NR₁₀—, —NR₁₀C(═NR₁₁)—, —C(═NR₁₁)NR₁₀—, —OC(═NR₁₁)—, or —C(═NR₁₁)O—;
   L₄ is optional, and when present is a bond, substituted or unsubstituted alkyl, substituted or unsubstituted cycloalkyl, substituted or unsubstituted alkenyl, substituted or unsubstituted alkynyl, substituted or unsubstituted aryl, substituted or unsubstituted heteroaryl, substituted or unsubstituted heterocycle;
   or L₃, X and L₄ taken together form a nitrogen containing heterocyclic ring;
   G is

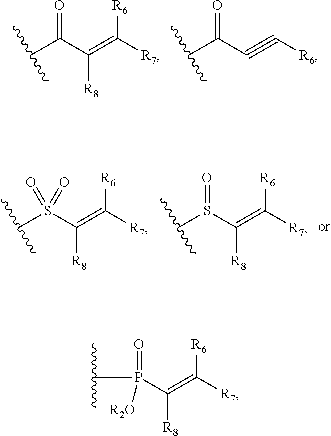

wherein,
   R₆, R₇ and R₈ are independently selected from among H, lower alkyl or substituted lower alkyl, lower heteroalkyl or substituted lower heteroalkyl, substituted or unsubstituted lower cycloalkyl, and substituted or unsubstituted lower heterocycloalkyl;
R₅ is H, halogen, -L₆-(substituted or unsubstituted C₁-C₃ alkyl), -L₆-(substituted or unsubstituted C₂-C₄ alkenyl), -L₆-(substituted or unsubstituted heteroaryl), or -L₆-(substituted or unsubstituted aryl), wherein L₆ is a bond, O, S, —S(═O), S(═O)₂, NH, C(O), —NHC(O)O, —OC(O)NH, —NHC(O), or —C(O)NH;
each R₉ is independently selected from among H, substituted or unsubstituted lower alkyl, and substituted or unsubstituted lower cycloalkyl;
each R₁₀ is independently H, substituted or unsubstituted lower alkyl, or substituted or unsubstituted lower cycloalkyl; or
two R₁₀ groups can together form a 5-, 6-, 7-, or 8-membered heterocyclic ring; or
R₁₀ and R₁₁ can together form a 5-, 6-, 7-, or 8-membered heterocyclic ring; or
each R₁₁ is independently selected from H or alkyl; and
   pharmaceutically active metabolites, pharmaceutically acceptable solvates,
   pharmaceutically acceptable salts, or pharmaceutically acceptable prodrugs thereof.
      In some embodiments, the compound of Formula A is (R)-1-(3-(4-amino-3-(4-phenoxyphenyl)-1H-pyrazolo[3,4-d]pyrimidin-1-yl)piperidin-1-yl)prop-2-en-1-one

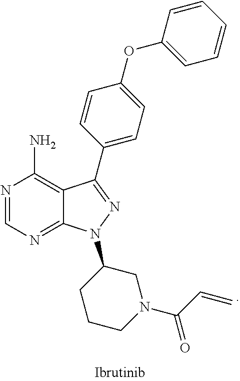 [SMILES: C=CC(=O)N1CCC[C@@H](n2nc(-c3ccc(Oc4ccccc4)cc3)c3c(N)ncnc32)C1 ; "."]

In some embodiments, the method prevents the onset of Type 1 Diabetes. In some embodiments, the method delays the onset of Type 1 Diabetes. In some embodiments, the number of anti-insulin B cells in the individual is reduced. In some embodiments, the individual is genetically predisposed to develop Type 1 Diabetes. In some embodiments, the individual presents with active autoimmunity to at least two of insulin, GAD65, IA-2, and Znt8. In some embodiments, the individual does not present with active autoimmunity to at least two of insulin, GAD65, IA-2, and Znt8. In some embodiments, the individual presents with an elevated level of A1C. In some embodiments, the individual does not present with an elevated level of A1C. In some embodiments, the individual presents with impaired fasting glycemia. In some embodiments, the individual presents with fasting plasma glucose level from 5.6 mmol/L (100 mg/dL) to 6.9 mmol/L (125 mg/dL). In some embodiments, the individual presents with fasting plasma glucose level from 6.1 mmol/L (100 mg/dL) to 6.9 mmol/L (125 mg/dL). In some embodiments, the individual does not present with impaired fasting glycemia. In some embodiments, the individual presents with impaired glucose tolerance. In some embodiments, the individual does not present with impaired glucose tolerance. In some embodiments, the individual is not insulin dependent. In some embodiments, the methods further comprise co-administering an additional therapeutic agent. In some embodiments, the additional therapeutic agent is cyclosporine A. In some embodiments, the additional therapeutic agent is an anti-CD3 antibody. In some embodiments, the anti-CD3 antibody is teplizumab, otelixizumab, visilizumab, or any combinations thereof.

Disclosed herein, in certain embodiments, are methods for preventing or delaying the onset of type 1 diabetes in an individual in need thereof comprising administering to the individual in need thereof a composition comprising a therapeutically-effective amount of (R)-1-(3-(4-amino-3-(4-phenoxyphenyl)-1H-pyrazolo[3,4-d]pyrimidin-1-yl)piperidin-1-yl)prop-2-en-1-one

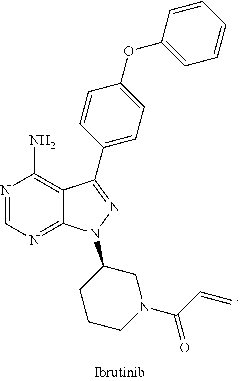 [SMILES: C=CC(=O)N1CCC[C@@H](n2nc(-c3ccc(Oc4ccccc4)cc3)c3c(N)ncnc32)C1 ; "."]

In some embodiments, the method prevents the onset of Type 1 Diabetes. In some embodiments, the method delays the onset of Type 1 Diabetes. In some embodiments, the number of anti-insulin B cells in the individual is reduced. In some embodiments, the individual is genetically predisposed to develop Type 1 Diabetes. In some embodiments, the individual presents with active autoimmunity to at least two of insulin, GAD65, IA-2, and Znt8. In some embodiments, the individual does not present with active autoimmunity to at least two of insulin, GAD65, IA-2, and Znt8. In some embodiments, the individual presents with an elevated level of A1C. In some embodiments, the individual does not present with an elevated level of A1C. In some embodiments, the individual presents with impaired fasting glycemia. In some embodiments, the individual presents with fasting plasma glucose level from 5.6 mmol/L (100 mg/dL) to 6.9 mmol/L (125 mg/dL). In some embodiments, the individual presents with fasting plasma glucose level from 6.1 mmol/L (100 mg/dL) to 6.9 mmol/L (125 mg/dL). In some embodiments, the individual does not present with impaired fasting glycemia. In some embodiments, the individual presents with impaired glucose tolerance. In some embodiments, the individual does not present with impaired glucose tolerance. In some embodiments, the individual is not insulin dependent. In some embodiments, the methods further comprise co-administering an additional therapeutic agent. In some embodiments, the additional therapeutic agent is cyclosporine A. In some embodiments, the additional therapeutic agent is an anti-CD3 antibody. In some embodiments, the anti-CD3 antibody is teplizumab, otelixizumab, visilizumab, or any combinations thereof.

Disclosed herein, in certain embodiments, are methods for inhibiting the maturation of anti-insulin B cells in an individual in need thereof comprising administering to the individual in need thereof a composition comprising a therapeutically-effective amount of a compound of Formula A:

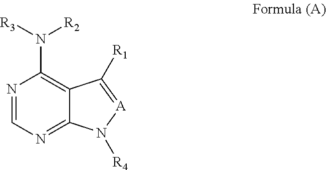

wherein
A is independently selected from N or CR₅;
R₁ is H, L₂-(substituted or unsubstituted alkyl), L₂-(substituted or unsubstituted cycloalkyl), L₂-(substituted or unsubstituted alkenyl), L₂-(substituted or unsubstituted cycloalkenyl), L₂-(substituted or unsubstituted heterocycle), L₂-(substituted or unsubstituted heteroaryl), or L₂-(substituted or unsubstituted aryl), where L₂ is a bond, O, S, —S(═O), —S(═O)₂, C(═O), -(substituted or unsubstituted C₁-C₆ alkyl), or -(substituted or unsubstituted C₂-C₆ alkenyl);
R₂ and R₃ are independently selected from H, lower alkyl and substituted lower alkyl;
R₄ is L₃-X-L₄-G, wherein,
   L₃ is optional, and when present is a bond, optionally substituted or unsubstituted alkyl, optionally substituted or unsubstituted cycloalkyl, optionally substituted or unsubstituted alkenyl, optionally substituted or unsubstituted alkynyl;
   X is optional, and when present is a bond, O, —C(═O), S, —S(═O), —S(═O)₂, —NH, —NR₉, —NHC(O), —C(O)NH, —NR₉C(O), —C(O)NR₉, —S(═O)₂NH, —NHS(═O)₂, —S(═O)₂NR₉—, —NR₉S(═O)₂, —OC(O)NH—, —NHC(O)O—, —OC(O)NR₉—, —NR₉C(O)O—, —CH═NO—, —ON═CH—, —NR₁₀C(O)NR₁₀—, heteroaryl, aryl, —NR₁₀C(═NR₁₁)NR₁₀—, —NR₁₀C(═NR₁₁)—, —C(═NR₁₁)NR₁₀—, —OC(═NR₁₁)—, or —C(═NR₁₁)O—;
   L₄ is optional, and when present is a bond, substituted or unsubstituted alkyl, substituted or unsubstituted cycloalkyl, substituted or unsubstituted alkenyl, substituted or unsubstituted alkynyl, substituted or unsubstituted aryl, substituted or unsubstituted heteroaryl, substituted or unsubstituted heterocycle;
   or L₃, X and L₄ taken together form a nitrogen containing heterocyclic ring;
   G is

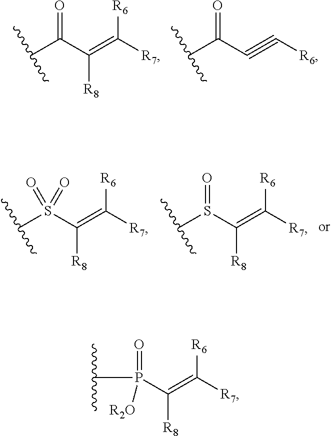

wherein,
   R₆, R₇ and R₈ are independently selected from among H, lower alkyl or substituted lower alkyl, lower heteroalkyl or substituted lower heteroalkyl, substituted or unsubstituted lower cycloalkyl, and substituted or unsubstituted lower heterocycloalkyl;
R₅ is H, halogen, -L₆-(substituted or unsubstituted C₁-C₃ alkyl), -L₆-(substituted or unsubstituted C₂-C₄ alkenyl), -L₆-(substituted or unsubstituted heteroaryl), or -L₆-(substituted or unsubstituted aryl), wherein L₆ is a bond, O, S, —S(═O), S(═O)₂, NH, C(O), —NHC(O)O, —OC(O)NH, —NHC(O), or —C(O)NH;
each R₉ is independently selected from among H, substituted or unsubstituted lower alkyl, and substituted or unsubstituted lower cycloalkyl;
each R₁₀ is independently H, substituted or unsubstituted lower alkyl, or substituted or unsubstituted lower cycloalkyl; or
two R₁₀ groups can together form a 5-, 6-, 7-, or 8-membered heterocyclic ring; or
R₁₀ and R₁₁ can together form a 5-, 6-, 7-, or 8-membered heterocyclic ring; or
each R₁₁ is independently selected from H or alkyl; and
   pharmaceutically active metabolites, pharmaceutically acceptable solvates,
   pharmaceutically acceptable salts, or pharmaceutically acceptable prodrugs thereof.
      In some embodiments, the compound of Formula A is (R)-1-(3-(4-amino-3-(4-phenoxyphenyl)-1H-pyrazolo[3,4-d]pyrimidin-1-yl)piperidin-1-yl)prop-2-en-1-one

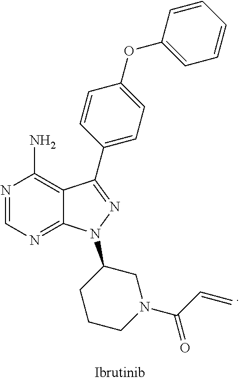 [SMILES: C=CC(=O)N1CCC[C@@H](n2nc(-c3ccc(Oc4ccccc4)cc3)c3c(N)ncnc32)C1 ; "."]

In some embodiments, the number of anti-insulin B cells in the individual is reduced. In some embodiments, the method prevents the onset of Type 1 Diabetes. In some embodiments, the method delays the onset of Type 1 Diabetes. In some embodiments, the individual is genetically predisposed to develop Type 1 Diabetes. In some embodiments, the individual presents with active autoimmunity to at least two of insulin, GAD65, IA-2, and Znt8. In some embodiments, the individual does not present with active autoimmunity to at least two of insulin, GAD65, IA-2, and Znt8. In some embodiments, the individual presents with an elevated level of A1C. In some embodiments, the individual does not present with an elevated level of A1C. In some embodiments, the individual presents with impaired fasting glycemia. In some embodiments, the individual presents with fasting plasma glucose level from 5.6 mmol/L (100 mg/dL) to 6.9 mmol/L (125 mg/dL). In some embodiments, the individual presents with fasting plasma glucose level from 6.1 mmol/L (100 mg/dL) to 6.9 mmol/L (125 mg/dL). In some embodiments, the individual does not present with impaired fasting glycemia. In some embodiments, the individual presents with impaired glucose tolerance. In some embodiments, the individual does not present with impaired glucose tolerance. In some embodiments, the individual is not insulin dependent. In some embodiments, the methods further comprise co-administering an additional therapeutic agent. In some embodiments, the additional therapeutic agent is cyclosporine A. In some embodiments, the additional therapeutic agent is an anti-CD3 antibody. In some embodiments, the anti-CD3 antibody is teplizumab, otelixizumab, visilizumab, or any combinations thereof.

Disclosed herein, in certain embodiments, are methods for inhibiting the maturation of anti-insulin B cells in an individual in need thereof comprising administering to the individual in need thereof a composition comprising a therapeutically-effective amount of (R)-1-(3-(4-amino-3-(4-phenoxyphenyl)-1H-pyrazolo[3,4-d]pyrimidin-1-yl)piperidin-1-yl)prop-2-en-1-one

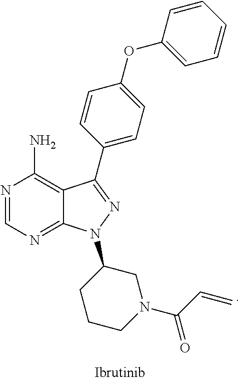 [SMILES: C=CC(=O)N1CCC[C@@H](n2nc(-c3ccc(Oc4ccccc4)cc3)c3c(N)ncnc32)C1 ; "."]

In some embodiments, the number of anti-insulin B cells in the individual is reduced. In some embodiments, the method prevents the onset of Type 1 Diabetes. In some embodiments, the method delays the onset of Type 1 Diabetes. In some embodiments, the individual is genetically predisposed to develop Type 1 Diabetes. In some embodiments, the individual presents with active autoimmunity to at least two of insulin, GAD65, IA-2, and Znt8. In some embodiments, the individual does not present with active autoimmunity to at least two of insulin, GAD65, IA-2, and Znt8. In some embodiments, the individual presents with an elevated level of A1C. In some embodiments, the individual does not present with an elevated level of A1C. In some embodiments, the individual presents with impaired fasting glycemia. In some embodiments, the individual presents with fasting plasma glucose level from 5.6 mmol/L (100 mg/dL) to 6.9 mmol/L (125 mg/dL). In some embodiments, the individual presents with fasting plasma glucose level from 6.1 mmol/L (100 mg/dL) to 6.9 mmol/L (125 mg/dL). In some embodiments, the individual does not present with impaired fasting glycemia. In some embodiments, the individual presents with impaired glucose tolerance. In some embodiments, the individual does not present with impaired glucose tolerance. In some embodiments, the individual is not insulin dependent. In some embodiments, the methods further comprise co-administering an additional therapeutic agent. In some embodiments, the additional therapeutic agent is cyclosporine A. In some embodiments, the additional therapeutic agent is an anti-CD3 antibody. In some embodiments, the anti-CD3 antibody is teplizumab, otelixizumab, visilizumab, or any combinations thereof.

Disclosed herein, in certain embodiments, are methods for decreasing the population of anti-insulin B cells in an individual in need thereof comprising administering to an individual in need thereof a composition comprising a therapeutically-effective amount of a covalent BTK inhibitor. In some embodiments, the covalent BTK inhibitor is an irreversible covalent BTK inhibitor. In some embodiments, the covalent BTK inhibitor is a compound of Formula A

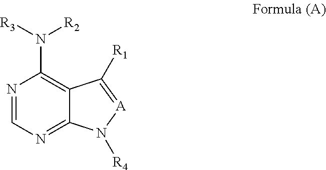

wherein
A is independently selected from N or CR₅;
R₁ is H, L₂-(substituted or unsubstituted alkyl), L₂-(substituted or unsubstituted cycloalkyl), L₂-(substituted or unsubstituted alkenyl), L₂-(substituted or unsubstituted cycloalkenyl), L₂-(substituted or unsubstituted heterocycle), L₂-(substituted or unsubstituted heteroaryl), or L₂-(substituted or unsubstituted aryl), where L₂ is a bond, O, S, —S(═O), —S(═O)₂, C(═O), -(substituted or unsubstituted C₁-C₆ alkyl), or -(substituted or unsubstituted C₂-C₆ alkenyl);
R₂ and R₃ are independently selected from H, lower alkyl and substituted lower alkyl;
R₄ is L₃-X-L₄-G, wherein,
   L₃ is optional, and when present is a bond, optionally substituted or unsubstituted alkyl, optionally substituted or unsubstituted cycloalkyl, optionally substituted or unsubstituted alkenyl, optionally substituted or unsubstituted alkynyl;
   X is optional, and when present is a bond, O, —C(═O), S, —S(═O), —S(═O)₂, —NH, —NR₉, —NHC(O), —C(O)NH, —NR₉C(O), —C(O)NR₉, —S(═O)₂NH, —NHS(═O)₂, —S(═O)₂NR₉—, —NR₉S(═O)₂, —OC(O)NH—, —NHC(O)O—, —OC(O)NR₉—, —NR₉C(O)O—, —CH═NO—, —ON═CH—, —NR₁₀C(O)NR₁₀—, heteroaryl, aryl, —NR₁₀C(═NR₁₁)NR₁₀—, —NR₁₀C(═NR₁₁)—, —C(═NR₁₁)NR₁₀—, —OC(═NR₁₁)—, or —C(═NR₁₁)O—;
   L₄ is optional, and when present is a bond, substituted or unsubstituted alkyl, substituted or unsubstituted cycloalkyl, substituted or unsubstituted alkenyl, substituted or unsubstituted alkynyl, substituted or unsubstituted aryl, substituted or unsubstituted heteroaryl, substituted or unsubstituted heterocycle;
   or L₃, X and L₄ taken together form a nitrogen containing heterocyclic ring;
   G is

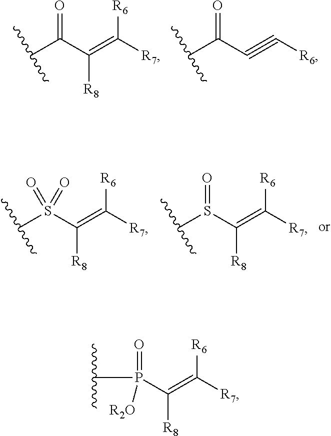

wherein,
   R₆, R₇ and R₈ are independently selected from among H, lower alkyl or substituted lower alkyl, lower heteroalkyl or substituted lower heteroalkyl, substituted or unsubstituted lower cycloalkyl, and substituted or unsubstituted lower heterocycloalkyl;
R₅ is H, halogen, -L₆-(substituted or unsubstituted C₁-C₃ alkyl), -L₆-(substituted or unsubstituted C₂-C₄ alkenyl), -L₆-(substituted or unsubstituted heteroaryl), or -L₆-(substituted or unsubstituted aryl), wherein L₆ is a bond, O, S, —S(═O), S(═O)₂, NH, C(O), —NHC(O)O, —OC(O)NH, —NHC(O), or —C(O)NH;
each R₉ is independently selected from among H, substituted or unsubstituted lower alkyl, and substituted or unsubstituted lower cycloalkyl;
each R₁₀ is independently H, substituted or unsubstituted lower alkyl, or substituted or unsubstituted lower cycloalkyl; or
two R₁₀ groups can together form a 5-, 6-, 7-, or 8-membered heterocyclic ring; or
R₁₀ and R₁₁ can together form a 5-, 6-, 7-, or 8-membered heterocyclic ring; or
each R₁₁ is independently selected from H or alkyl; and
   pharmaceutically active metabolites, pharmaceutically acceptable solvates,
   pharmaceutically acceptable salts, or pharmaceutically acceptable prodrugs thereof.
      In some embodiments, the compound of Formula (A) has the structure:

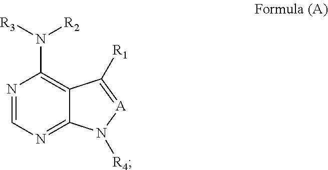

wherein:
A is N;
R₂ and R₃ are each H;
R₁ is phenyl-O-phenyl or phenyl-S-phenyl; and
R₄ is L₃-X-L₄-G, wherein,
   L₃ is optional, and when present is a bond, optionally substituted or unsubstituted alkyl, optionally substituted or unsubstituted cycloalkyl, optionally substituted or unsubstituted alkenyl, optionally substituted or unsubstituted alkynyl;
   X is optional, and when present is a bond, O, —C(═O), S, —S(═O), —S(═O)₂, —NH, —NR₉, —NHC(O), —C(O)NH, —NR₉C(O), —C(O)NR₉, —S(═O)₂NH, —NHS(═O)₂, —S(═O)₂NR₉—, —NR₉S(═O)₂, —OC(O)NH—, —NHC(O)O—, —OC(O)NR₉—, —NR₉C(O)O—, —CH═NO—, —ON═CH—, —NR₁₀C(O)NR₁₀—, heteroaryl, aryl, —NR₁₀C(═NR₁₁)NR₁₀—, —NR₁₀C(═NR₁₁)—, —C(═NR₁₁)NR₁₀—, —OC(═NR₁₁)—, or —C(═NR₁₁)O—;
   L₄ is optional, and when present is a bond, substituted or unsubstituted alkyl, substituted or unsubstituted cycloalkyl, substituted or unsubstituted alkenyl, substituted or unsubstituted alkynyl, substituted or unsubstituted aryl, substituted or unsubstituted heteroaryl, substituted or unsubstituted heterocycle;
   or L₃, X and L₄ taken together form a nitrogen containing heterocyclic ring;
   G is

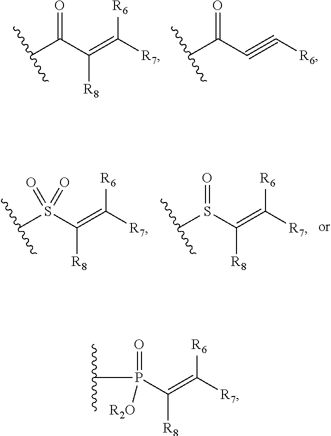

wherein,
R₆, R₇ and R₈ are independently selected from among H, lower alkyl or substituted lower alkyl, lower heteroalkyl or substituted lower heteroalkyl, substituted or unsubstituted lower cycloalkyl, and substituted or unsubstituted lower heterocycloalkyl.
   In some embodiments, the covalent BTK inhibitor is (R)-1-(3-(4-amino-3-(4-phenoxyphenyl)-1H-pyrazolo[3,4-d]pyrimidin-1-yl)piperidin-1-yl)prop-2-en-1-one

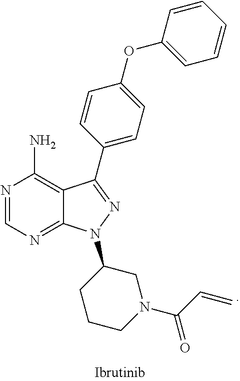 [SMILES: C=CC(=O)N1CCC[C@@H](n2nc(-c3ccc(Oc4ccccc4)cc3)c3c(N)ncnc32)C1 ; "."]

In some embodiments, the anti-insulin B cells are mature anti-insulin B cells. In some embodiments, the population of mature anti-insulin B cells is reduced. In some embodiments, the method delays the onset of Type 1 Diabetes. In some embodiments, the method reduces the severity of Type 1 Diabetes. In some embodiments, the method prevents the development of Type 1 Diabetes. In some embodiments, the method further comprises administering an additional therapeutic agent. In some embodiments, the additional therapeutic agent is cyclosporine A. In some embodiments, the additional therapeutic agent is an anti-CD3 antibody. In some embodiments, the anti-CD3 antibody is teplizumab or otelixizumab.

Disclosed herein, in certain embodiments, are methods for decreasing the population of anti-insulin B cells in an individual in need thereof comprising administering to an individual in need thereof a composition comprising a therapeutically-effective amount of (R)-1-(3-(4-amino-3-(4-phenoxyphenyl)-1H-pyrazolo[3,4-d]pyrimidin-1-yl)piperidin-1-yl)prop-2-en-1-one

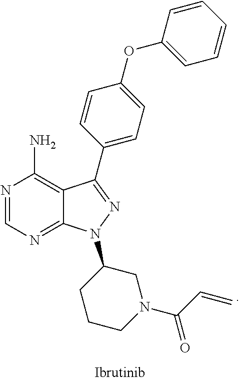 [SMILES: C=CC(=O)N1CCC[C@@H](n2nc(-c3ccc(Oc4ccccc4)cc3)c3c(N)ncnc32)C1 ; "."]

In some embodiments, the anti-insulin B cells are mature anti-insulin B cells. In some embodiments, the population of mature anti-insulin B cells is reduced. In some embodiments, the method delays the onset of Type 1 Diabetes. In some embodiments, the method reduces the severity of Type 1 Diabetes. In some embodiments, the method prevents the development of Type 1 Diabetes. In some embodiments, the method further comprises administering an additional therapeutic agent. In some embodiments, the additional therapeutic agent is cyclosporine A. In some embodiments, the additional therapeutic agent is an anti-CD3 antibody. In some embodiments, the anti-CD3 antibody is teplizumab or otelixizumab.

### BRIEF DESCRIPTION OF THE FIGURES

FIG. 1 exemplifies the effects of Btk-deficiency on anti-insulin B cells and An1 cells in the spleen. Panels A and B illustrate the expression of B220, IgM, and insulin-reactivity was assessed in 125Tg/NOD Btk-sufficient or Btk-deficient splenocytes using flow cytometry. Representative flow cytometry plots are shown in A. Left panel is gated on live lymphocytes, Right panel is gated on B220⁺ IgM^{a+} live lymphocytes. The average number of B cells is shown ±SEM, Btk-sufficient (black), Btk-deficient (white) are shown in B. Panel C shows splenocytes were harvested and CD93+ cells were identified in B220⁺ IgM⁺ live lymphocytes (left); CD93⁺ gated B cells were further divided into the An1 subset (IgM^{lo} CD23⁺) from WT/C57BL/6 Btk-sufficient or Btk-deficient mice (right). Panel D shows the average percentage of An1 cells among total B cells (left) or the total number of An1 cells (right) is indicated for Btk-sufficient (black) or Btk-deficient (white) mice. (Panels A-B) n≧10 8-15 week old male and female mice per group, n=3 experiments, (Panels C-D) n≧7 8-10 week old male and female mice per group, n=2 experiments, *p<0.01, **p<0.001 as calculated by a two-tailed t-test. All mice had a blood glucose <200 mg/dL.

FIG. 2A-FIG. 2D demonstrate that anti-insulin immature B cells do not require BTK to develop or to mobilize calcium following BCR stimulation. FIG. 2A shows representative flow cytometry dotplots of bone marrow isolates from Btk-sufficient or Btk-deficient 125Tg/NOD mice: Left panel is gated on B220⁺ live lymphocytes. Middle and Right panels are gated on B220⁺ IgMa⁺ live lymphocytes. The average percentages (FIG. 2B) or total numbers (FIG. 2C) of pro/pre (IgM^{a-}), immature (IgM^{a+} CD23⁻), or mature recirculating (IgM^{a+} CD23⁺) B cells (B220⁺ live lymphocytes) are shown ±SEM. Btk-sufficient (black), Btk-deficient (white), n≧8 male and female mice 9-16 weeks of age, n=4 experiments, *p<0.05, **p<0.01, ***p<0.001, as calculated by a two-tailed t-test. FIG. 2D shows bone marrow cells from 125Tg/NOD Btk-sufficient (solid line) or deficient (dashed line) mice were cultured with IL-7 for 5 d, then 2 d without IL-7 to generate naïve immature B cells. Cells were stimulated and calcium mobilization was measured as in Methods. Arrow indicates stimulation with 1 μg/mL anti-IgM. Data are representative of n≧4 mice, n=2 experiments.

FIG. 3A-FIG. 3C exemplify the effects of Btk-deficiency on the numbers of anti-insulin B cells various all developmental stages, including mature subsets. Flow cytometry was used to analyze splenic B cell subsets in 125Tg/NOD mice. FIG. 3A shows representative flow cytometry dotplots show the gating scheme for T1, T2, follicular, pre-marginal zone and marginal zone B cells from Btk-sufficient (upper panels) and Btk-deficient (lower panels) 125Tg/NOD mice. Left: B220⁺ IgM^{a+} live lymphocytes, Right: B220⁺ IgM^{a+} CD21^{mid} CD23^{high} live lymphocytes. The average percentages (FIG. 3B) or total numbers (FIG. 3C) of T1 (CD21^{low} CD23^{low}), T2 (CD21^{mid} cD23^{high}), follicular (CD21^{mid} CD23^{high}, IgM^{mid}), pre-marginal zone (CD21^{high}, CD23^{high}, IgM^{high}), and marginal zone (CD21^{high} CD23^{mid}) B cells (B220⁺, IgM⁺ live lymphocytes) are shown ±SEM, for Btk-sufficient (black) and Btk-deficient (white) 125Tg/NOD mice, (FIG. 3A-3B) n≧10 8-15 week old male and female mice per group, n=3 experiments, *p<0.05, **p<0.01, ***p<0.001, as calculated by a two-tailed t-test. All mice had a blood glucose <200 mg/dL.

FIG. 4A-FIG. 4C exemplify the effects of Ibrutinib, a BTK kinase inhibitor, on the numbers of mature anti-insulin B cells in the spleen, draining pancreatic lymph nodes and pancreas. 125Tg/NOD mice were fed 0.24 g Ibrutinib/kg feed, for an average of 38 mg inhibitor/kg consumed per day per mouse. After 5-10 weeks, spleen, draining pancreatic lymph nodes, and pancreas were harvested and flow cytometry was used to analyze B cell subsets. FIG. 4A shows the number of B lymphocytes (B220⁺ IgM⁺ live lymphocytes) and non-B lymphocytes (B220⁻ IgM⁻ live lymphocytes) was assessed in spleens of n=6 placebo (black) and n=7 inhibitor (white) treated male and female mice. FIG. 4B show spleen B cell subsets that were identified as in FIG. 3 and subset numbers were determined for n=3 placebo (black) and n=3 inhibitor (white) treated mice. FIG. 4C shows B cell numbers that were assessed by flow cytometry in freshly isolated pancreatic draining lymph nodes and pancreas of n=3 female mice per group, placebo (black), inhibitor (white). Mice were 12-13 weeks old at time of analysis. Error bars indicate standard deviation, **p<0.01, ***p<0.001, as calculated by a two-tailed t-test.

FIG. 5A-FIG. 5D exemplify the effects of an absence of Btk on anti-insulin B cell transition into follicular and marginal zone B cells. V_{H}125Tg/Vκ125^{SD}/NOD mice were developed as in Methods and bone marrow and splenocyte B cell subsets were identified as in FIG. 3. FIG. 5A shows representative flow cytometry plots of B220⁺ IgM^{a+} gated live lymphocytes from spleens, showing insulin-binding by staining with labeled insulin. Average percentages (FIG. 5B) or total numbers (FIG. 5C) of insulin-binding B cells present in immature, T1, T2, follicular, pre-marginal zone, and marginal zone subsets are shown ±SEM, Btk-sufficient (black), Btk-deficient (white). FIG. 5D shows the fold change in the number of insulin-binding B cells present in each subset from FIG. 5C was calculated by dividing the average number of B cells in the Btk-sufficient subset by the average number in the comparable subset from Btk-sufficient mice (or the reverse for fold increase), shown in grey. The same was also calculated for non-insulin-binding B cells (white). Dashed lines=1 (no change). n≧9 mice, n≧2 experiments, *p<0.05, **p<0.01, ***p<0.001, as calculated by a two-tailed t-test.

FIG. 6A-FIG. 6D exemplify Btk-mediated effects on factors related to B cell trafficking in VH125Tg/Vκ125SD/NOD mice. FIG. 6A-FIG. 6B illustrate sinusoidal positioning for bone-marrow exit: Anti-CD19-PE antibody was injected i.v. and V_{H}125Tg/Vκ125^{SD}/NOD mice were sacrificed after 2 min to preferentially label sinusoidal B cells as in Methods. Bone marrow was immediately harvested and antibodies reactive with B220, IgM, CD19-APC, and CD23 were used to identify immature B cells (left panels) that were divided into parenchymal (CD19-PE^{mid}, middle panels) and sinusoidal (CD19-PE^{high}, right panels) B cell populations. Insulin-binding B cells were further identified using labeled insulin. FIG. 6A shows representative flow cytometry plots and FIG. 6B shows the average percentages of insulin-binding parenchymal or sinusoidal immature B cells which are shown as ±SD, Btk-sufficient (black), Btk-deficient (white), n≧5 mice, n=2 experiments. FIG. 6C-FIG. 6D shows flow cytometry using labeled insulin which was used to assess proportion (FIG. 6C) and number (FIG. 6D) of insulin-binding B cells in pancreas and draining pancreatic lymph nodes (live B220⁺ IgM^{a+} lymphocytes), and follicular spleen B cells (gated as in FIG. 3) in Btk-sufficient (black) or Btk-deficient (white) mice. The average percentage ±SEM of insulin-binding B cells, n≧6 female mice, n≧3 experiments. *p<0.05, **p<0.01, ***p<0.001, as calculated by a two-tailed t-test.

FIG. 7A-FIG. 7C exemplify the effects of Btk deficiency on B cell internalization of insulin autoantigen and capability for supporting diabetes relative to their BTK-sufficient counterparts. FIG. 7A shows 125Tg/NOD Btk-sufficient or Btk-deficient bone marrow or spleens were harvested, cells were loaded with 50 ng/mL biotinylated insulin in media on ice, were washed, then placed at 37° C. for 0-10 min Cells were then stained on ice with streptavidin-fluorochrome to detect remaining surface insulin as well as with antibodies reactive with B220, IgM, CD23 (bone marrow), and CD21 (spleen) to identify developmental subsets as in FIG. 2-3. The MFI of insulin-biotin/streptavidin-fluorochrome (or IgM) at each time point was divided by the insulin-biotin/streptavidin-fluorochrome (or IgM) MFI at t=0, such that 0 min=100% expression for insulin internalization (or Relative Surface IgM). Insulin Internalization (solid lines) and Relative Surface IgM (dashed lines) is plotted. The average ± standard deviation is shown for n=4 Btk-sufficient and n=6 Btk-deficient male and female mice 8-13 wks of age, n=3 experiments. Statistical significance was calculated using a two-tailed t-test for Btk-sufficient vs. Btk-deficient at each time point, *p<0.05. FIG. 7B shows flow cytometry which was used to assess proportion of total (left) and number (right) of insulin-binding B cells in draining pancreatic lymph nodes and pancreas (live B220+ IgMa+ lymphocytes) in Btk-sufficient (black) or Btk-deficient (white) n≧9 female mice. FIG. 7C shows diabetes onset which was monitored in 125Tg/NOD Btk-sufficient (n=12), 125Tg/NOD Btk-deficient (n=15), WT/NOD Btk-sufficient (n=9), or WT/NOD Btk-deficient (n=8) female mice. Mice were considered diabetic after two consecutive blood glucose readings >200 mg/dL. p=0.045 for Non-Tg/NOD Btk-deficient vs. 125Tg/NOD Btk-deficient, p=0.235 for 125Tg/NOD Btk-deficient vs. 125Tg/NOD Btk-sufficient using a log rank test.

### DETAILED DESCRIPTION OF THE INVENTION

Disclosed herein, in certain embodiments, are methods for preventing or delaying the onset of Type 1 Diabetes in an individual in need thereof comprising administering to an individual in need thereof a composition comprising a therapeutically-effective amount of a covalent BTK inhibitor. In some embodiments, the covalent BTK inhibitor is an irreversible covalent BTK inhibitor. In some embodiments, the covalent BTK inhibitor is a compound of Formula A

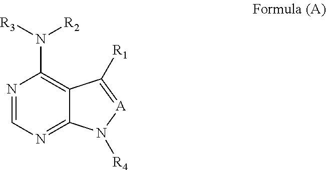

wherein
   A is independently selected from N or CR₅;
   R₁ is H, L₂-(substituted or unsubstituted alkyl), L₂-(substituted or unsubstituted cycloalkyl), L₂-(substituted or unsubstituted alkenyl), L₂-(substituted or unsubstituted cycloalkenyl), L₂-(substituted or unsubstituted heterocycle), L₂-(substituted or unsubstituted heteroaryl), or L₂-(substituted or unsubstituted aryl), where L₂ is a bond, O, S, —S(═O), —S(═O)₂, C(═O), -(substituted or unsubstituted C₁-C₆ alkyl), or -(substituted or unsubstituted C₂-C₆ alkenyl);
   R₂ and R₃ are independently selected from H, lower alkyl and substituted lower alkyl;
   R₄ is L₃-X-L₄-G, wherein,
      L₃ is optional, and when present is a bond, optionally substituted or unsubstituted alkyl, optionally substituted or unsubstituted cycloalkyl, optionally substituted or unsubstituted alkenyl, optionally substituted or unsubstituted alkynyl;
      X is optional, and when present is a bond, O, —C(═O), S, —S(═O), —S(═O)₂, —NH, —NR₉, —NHC(O), —C(O)NH, —NR₉C(O), —C(O)NR₉, —S(═O)₂NH, —NHS(═O)₂, —S(═O)₂NR₉—, —NR₉S(═O)₂, —OC(O)NH—, —NHC(O)O—, —OC(O)NR₉—, —NR₉C(O)O—, —CH═NO—, —ON═CH—, —NR₁₀C(O)NR₁₀—, heteroaryl, aryl, —NR₁₀C(═NR₁₁)NR₁₀—, —NR₁₀C(═NR₁₁)—, —C(═NR₁₁)NR₁₀—, —OC(═NR₁₁)—, or —C(═NR₁₁)O—;
      L₄ is optional, and when present is a bond, substituted or unsubstituted alkyl, substituted or unsubstituted cycloalkyl, substituted or unsubstituted alkenyl, substituted or unsubstituted alkynyl, substituted or unsubstituted aryl, substituted or unsubstituted heteroaryl, substituted or unsubstituted heterocycle;
      or L₃, X and L₄ taken together form a nitrogen containing heterocyclic ring;
      G is

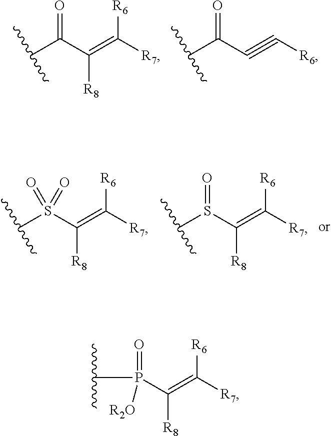

wherein,
   R₆, R₇ and R₈ are independently selected from among H, lower alkyl or substituted lower alkyl, lower heteroalkyl or substituted lower heteroalkyl, substituted or unsubstituted lower cycloalkyl, and substituted or unsubstituted lower heterocycloalkyl;
R₅ is H, halogen, -L₆-(substituted or unsubstituted C₁-C₃ alkyl), -L₆-(substituted or unsubstituted C₂-C₄ alkenyl), -L₆-(substituted or unsubstituted heteroaryl), or -L₆-(substituted or unsubstituted aryl), wherein L₆ is a bond, O, S, —S(═O), S(═O)₂, NH, C(O), —NHC(O)O, —OC(O)NH, —NHC(O), or —C(O)NH;
each R₉ is independently selected from among H, substituted or unsubstituted lower alkyl, and substituted or unsubstituted lower cycloalkyl;
each R₁₀ is independently H, substituted or unsubstituted lower alkyl, or substituted or unsubstituted lower cycloalkyl; or
two R₁₀ groups can together form a 5-, 6-, 7-, or 8-membered heterocyclic ring; or
R₁₀ and R₁₁ can together form a 5-, 6-, 7-, or 8-membered heterocyclic ring; or
each R₁₁ is independently selected from H or alkyl; and
pharmaceutically active metabolites, pharmaceutically acceptable solvates, pharmaceutically acceptable salts, or pharmaceutically acceptable prodrugs thereof.
   In some embodiments, the compound of Formula (A) has the structure:

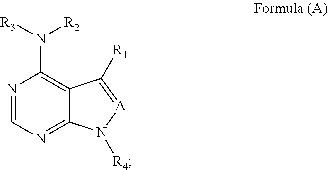

wherein:
A is N;
R₂ and R₃ are each H;
R₁ is phenyl-O-phenyl or phenyl-S-phenyl; and
R₄ is L₃-X-L₄-G, wherein,
   L₃ is optional, and when present is a bond, optionally substituted or unsubstituted alkyl, optionally substituted or unsubstituted cycloalkyl, optionally substituted or unsubstituted alkenyl, optionally substituted or unsubstituted alkynyl;
   X is optional, and when present is a bond, O, —C(═O), S, —S(═O), —S(═O)₂, —NH, —NR₉, —NHC(O), —C(O)NH, —NR₉C(O), —C(O)NR₉, —S(═O)₂NH, —NHS(═O)₂, —S(═O)₂NR₉—, —NR₉S(═O)₂, —OC(O)NH—, —NHC(O)O—, —OC(O)NR₉—, —NR₉C(O)O—, —CH═NO—, —ON═CH—, —NR₁₀C(O)NR₁₀—, heteroaryl, aryl, —NR₁₀C(═NR₁₁)NR₁₀—, —NR₁₀C(═NR₁₁)—, —C(═NR₁₁)NR₁₀—, —OC(═NR₁₁)—, or —C(═NR₁₁)O—;
   L₄ is optional, and when present is a bond, substituted or unsubstituted alkyl, substituted or unsubstituted cycloalkyl, substituted or unsubstituted alkenyl, substituted or unsubstituted alkynyl, substituted or unsubstituted aryl, substituted or unsubstituted heteroaryl, substituted or unsubstituted heterocycle;
   or L₃, X and L₄ taken together form a nitrogen containing heterocyclic ring;
   G is

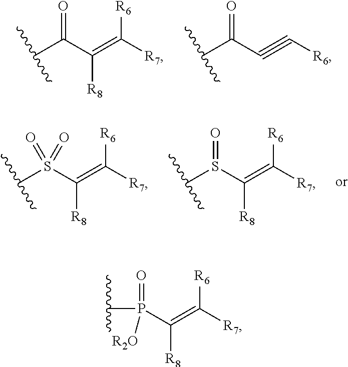

wherein,
R₆, R₇ and R₈ are independently selected from among H, lower alkyl or substituted lower alkyl, lower heteroalkyl or substituted lower heteroalkyl, substituted or unsubstituted lower cycloalkyl, and substituted or unsubstituted lower heterocycloalkyl.
   In some embodiments, the covalent BTK inhibitor is (R)-1-(3-(4-amino-3-(4-phenoxyphenyl)-1H-pyrazolo[3,4-d]pyrimidin-1-yl)piperidin-1-yl)prop-2-en-1-one

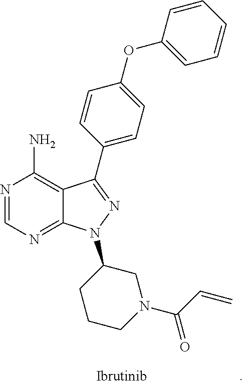 [SMILES: C=CC(=O)N1CCC[C@@H](n2nc(-c3ccc(Oc4ccccc4)cc3)c3c(N)ncnc32)C1]

In some embodiments, the number of anti-insulin B cells is reduced. In some embodiments, the method prevents the onset of Type 1 Diabetes. In some embodiments, the method delays the onset of Type 1 Diabetes. In some embodiments, the method further comprises administering an additional therapeutic agent. In some embodiments, the additional therapeutic agent is cyclosporine A. In some embodiments, the additional therapeutic agent is an anti-CD3 antibody. In some embodiments, the anti-CD3 antibody is teplizumab or otelixizumab.

Disclosed herein, in certain embodiments, are methods for preventing or delaying the onset of Type 1 Diabetes in an individual in need thereof comprising administering to an individual in need thereof a composition comprising a therapeutically-effective amount of (R)-1-(3-(4-amino-3-(4-phenoxyphenyl)-1H-pyrazolo[3,4-d]pyrimidin-1-yl)piperidin-1-yl)prop-2-en-1-one

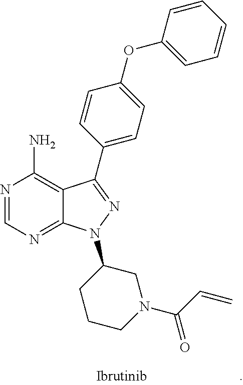 [SMILES: C=CC(=O)N1CCC[C@@H](n2nc(-c3ccc(Oc4ccccc4)cc3)c3c(N)ncnc32)C1]

In some embodiments, the number of anti-insulin B cells is reduced. In some embodiments, the method delays the onset of Type 1 Diabetes. In some embodiments, the method prevents the onset of Type 1 Diabetes. In some embodiments, the method further comprises administering an additional therapeutic agent. In some embodiments, the additional therapeutic agent is cyclosporine A. In some embodiments, the additional therapeutic agent is an anti-CD3 antibody. In some embodiments, the anti-CD3 antibody is teplizumab or otelixizumab.

Disclosed herein, in certain embodiments, are methods for inhibiting the maturation of anti-insulin B cells in an individual in need thereof comprising administering to an individual in need thereof a composition comprising a therapeutically-effective amount of a covalent BTK inhibitor. In some embodiments, the covalent BTK inhibitor is an irreversible covalent BTK inhibitor. In some embodiments, the covalent BTK inhibitor is a compound of Formula A

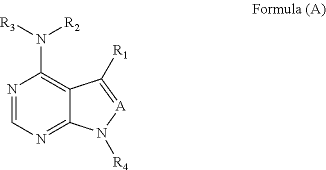

wherein
   A is independently selected from N or CR₅;
   R₁ is H, L₂-(substituted or unsubstituted alkyl), L₂-(substituted or unsubstituted cycloalkyl), L₂-(substituted or unsubstituted alkenyl), L₂-(substituted or unsubstituted cycloalkenyl), L₂-(substituted or unsubstituted heterocycle), L₂-(substituted or unsubstituted heteroaryl), or L₂-(substituted or unsubstituted aryl), where L₂ is a bond, O, S, —S(═O), —S(═O)₂, C(═O), -(substituted or unsubstituted C₁-C₆ alkyl), or -(substituted or unsubstituted C₂-C₆alkenyl);
   R₂ and R₃ are independently selected from H, lower alkyl and substituted lower alkyl;
   R₄ is L₃-X-L₄-G, wherein,
      L₃ is optional, and when present is a bond, optionally substituted or unsubstituted alkyl, optionally substituted or unsubstituted cycloalkyl, optionally substituted or unsubstituted alkenyl, optionally substituted or unsubstituted alkynyl;
      X is optional, and when present is a bond, O, —C(═O), S, —S(═O), —S(═O)₂, —NH, —NR₉, —NHC(O), —C(O)NH, —NR₉C(O), —C(O)NR₉, —S(═O)₂NH, —NHS(═O)₂, —S(═O)₂NR₉—, —NR₉S(═O)₂, —OC(O)NH—, —NHC(O)O—, —OC(O)NR₉—, —NR₉C(O)O—, —CH═NO—, —ON═CH—, —NR₁₀C(O)NR₁₀—, heteroaryl, aryl, —NR₁₀C(═NR₁₁)NR₁₀—, —NR₁₀C(═NR₁₁)—, —C(═NR₁₁)NR₁₀—, —OC(═NR₁₁)—, or —C(═NR₁₁)O—;
      L₄ is optional, and when present is a bond, substituted or unsubstituted alkyl, substituted or unsubstituted cycloalkyl, substituted or unsubstituted alkenyl, substituted or unsubstituted alkynyl, substituted or unsubstituted aryl, substituted or unsubstituted heteroaryl, substituted or unsubstituted heterocycle;
      or L₃, X and L₄ taken together form a nitrogen containing heterocyclic ring;
      G is

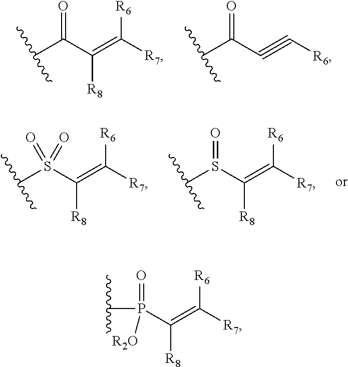

wherein,
   R₆, R₇ and R₈ are independently selected from among H, lower alkyl or substituted lower alkyl, lower heteroalkyl or substituted lower heteroalkyl, substituted or unsubstituted lower cycloalkyl, and substituted or unsubstituted lower heterocycloalkyl;
R₅ is H, halogen, -L₆-(substituted or unsubstituted C₁-C₃ alkyl), -L₆-(substituted or unsubstituted C₂-C₄ alkenyl), -L₆-(substituted or unsubstituted heteroaryl), or -L₆-(substituted or unsubstituted aryl), wherein L₆ is a bond, O, S, —S(═O), S(═O)₂, NH, C(O), —NHC(O)O, —OC(O)NH, —NHC(O), or —C(O)NH;
each R₉ is independently selected from among H, substituted or unsubstituted lower alkyl, and substituted or unsubstituted lower cycloalkyl;
each R₁₀ is independently H, substituted or unsubstituted lower alkyl, or substituted or unsubstituted lower cycloalkyl; or
two R₁₀ groups can together form a 5-, 6-, 7-, or 8-membered heterocyclic ring; or
R₁₀ and R₁₁ can together form a 5-, 6-, 7-, or 8-membered heterocyclic ring; or
each R₁₁ is independently selected from H or alkyl; and
pharmaceutically active metabolites, pharmaceutically acceptable solvates, pharmaceutically acceptable salts, or pharmaceutically acceptable prodrugs thereof.
   In some embodiments, the compound of Formula (A) has the structure:

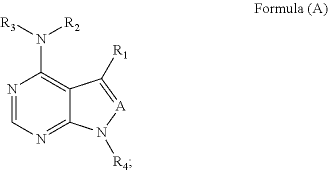

wherein:
A is N;
R₂ and R₃ are each H;
R₁ is phenyl-O-phenyl or phenyl-S-phenyl; and
R₄ is L₃-X-L₄-G, wherein,
   L₃ is optional, and when present is a bond, optionally substituted or unsubstituted alkyl, optionally substituted or unsubstituted cycloalkyl, optionally substituted or unsubstituted alkenyl, optionally substituted or unsubstituted alkynyl;
   X is optional, and when present is a bond, O, —C(═O), S, —S(═O), —S(═O)₂, —NH, —NR₉, —NHC(O), —C(O)NH, —NR₉C(O), —C(O)NR₉, —S(═O)₂NH, —NHS(═O)₂, —S(═O)₂NR₉—, —NR₉S(═O)₂, —OC(O)NH—, —NHC(O)O—, —OC(O)NR₉—, —NR₉C(O)O—, —CH═NO—, —ON═CH—, —NR₁₀C(O)NR₁₀—, heteroaryl, aryl, —NR₁₀C(═NR₁₁)NR₁₀—, —NR₁₀C(═NR₁₁)—, —C(═NR₁₁)NR₁₀—, —OC(═NR₁₁)—, or —C(═NR₁₁)O—;
   L₄ is optional, and when present is a bond, substituted or unsubstituted alkyl, substituted or unsubstituted cycloalkyl, substituted or unsubstituted alkenyl, substituted or unsubstituted alkynyl, substituted or unsubstituted aryl, substituted or unsubstituted heteroaryl, substituted or unsubstituted heterocycle;
   or L₃, X and L₄ taken together form a nitrogen containing heterocyclic ring;
   G is

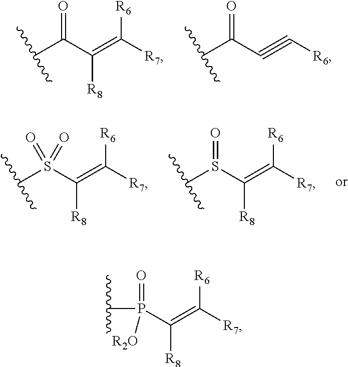

wherein,
R₆, R₇ and R₈ are independently selected from among H, lower alkyl or substituted lower alkyl, lower heteroalkyl or substituted lower heteroalkyl, substituted or unsubstituted lower cycloalkyl, and substituted or unsubstituted lower heterocycloalkyl.
   In some embodiments, the covalent BTK inhibitor is (R)-1-(3-(4-amino-3-(4-phenoxyphenyl)-1H-pyrazolo[3,4-d]pyrimidin-1-yl)piperidin-1-yl)prop-2-en-1-one

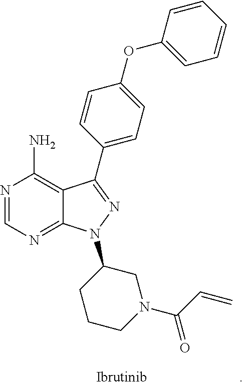 [SMILES: C=CC(=O)N1CCC[C@@H](n2nc(-c3ccc(Oc4ccccc4)cc3)c3c(N)ncnc32)C1]

In some embodiments, the number of anti-insulin B cells is reduced. In some embodiments, the method delays the onset of Type 1 Diabetes. In some embodiments, the method reduces the severity of Type 1 Diabetes. In some embodiments, the method prevents the development of Type 1 Diabetes. In some embodiments, the method further comprises administering an additional therapeutic agent. In some embodiments, the additional therapeutic agent is cyclosporine A. In some embodiments, the additional therapeutic agent is an anti-CD3 antibody. In some embodiments, the anti-CD3 antibody is teplizumab or otelixizumab.

Disclosed herein, in certain embodiments, are methods for inhibiting the maturation of anti-insulin B cells in an individual in need thereof comprising administering to an individual in need thereof a composition comprising a therapeutically-effective amount of (R)-1-(3-(4-amino-3-(4-phenoxyphenyl)-1H-pyrazolo[3,4-d]pyrimidin-1-yl)piperidin-1-yl)prop-2-en-1-one

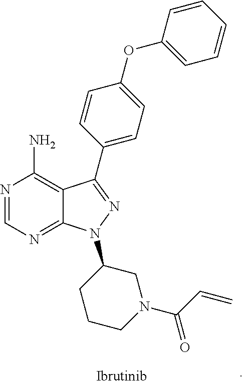 [SMILES: C=CC(=O)N1CCC[C@@H](n2nc(-c3ccc(Oc4ccccc4)cc3)c3c(N)ncnc32)C1]

In some embodiments, the number of mature anti-insulin B cells is reduced. In some embodiments, the method delays the onset of Type 1 Diabetes. In some embodiments, the method reduces the severity of Type 1 Diabetes. In some embodiments, the method prevents the development of Type 1 Diabetes. In some embodiments, the method further comprises administering an additional therapeutic agent. In some embodiments, the additional therapeutic agent is cyclosporine A. In some embodiments, the additional therapeutic agent is an anti-CD3 antibody. In some embodiments, the anti-CD3 antibody is teplizumab or otelixizumab.

Disclosed herein, in certain embodiments, are methods for decreasing the population of anti-insulin B cells in an individual in need thereof comprising administering to an individual in need thereof a composition comprising a therapeutically-effective amount of a covalent BTK inhibitor. In some embodiments, the covalent BTK inhibitor is an irreversible covalent BTK inhibitor. In some embodiments, the covalent BTK inhibitor is a compound of Formula A

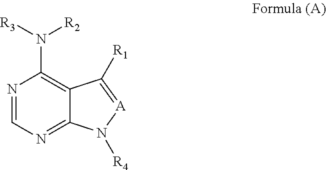

wherein
A is independently selected from N or CR₅;
R₁ is H, L₂-(substituted or unsubstituted alkyl), L₂-(substituted or unsubstituted cycloalkyl), L₂-(substituted or unsubstituted alkenyl), L₂-(substituted or unsubstituted cycloalkenyl), L₂-(substituted or unsubstituted heterocycle), L₂-(substituted or unsubstituted heteroaryl), or L₂-(substituted or unsubstituted aryl), where L₂ is a bond, O, S, —S(═O), —S(═O)₂, C(═O), -(substituted or unsubstituted C₁-C₆ alkyl), or -(substituted or unsubstituted C₂-C₆ alkenyl);
R₂ and R₃ are independently selected from H, lower alkyl and substituted lower alkyl;
R₄ is L₃-X-L₄-G, wherein,
   L₃ is optional, and when present is a bond, optionally substituted or unsubstituted alkyl, optionally substituted or unsubstituted cycloalkyl, optionally substituted or unsubstituted alkenyl, optionally substituted or unsubstituted alkynyl;
   X is optional, and when present is a bond, O, —C(═O), S, —S(═O), —S(═O)₂, —NH, —NR₉, —NHC(O), —C(O)NH, —NR₉C(O), —C(O)NR₉, —S(═O)₂NH, —NHS(═O)₂, —S(═O)₂NR₉—, —NR₉S(═O)₂, —OC(O)NH—, —NHC(O)O—, —OC(O)NR₉—, —NR₉C(O)O—, —CH═NO—, —ON═CH—, —NR₁₀C(O)NR₁₀—, heteroaryl, aryl, —NR₁₀C(═NR₁₁)NR₁₀—, —NR₁₀C(═NR₁₁)—, —C(═NR₁₁)NR₁₀—, —OC(═NR₁₁)—, or —C(═NR₁₁)O—;
   L₄ is optional, and when present is a bond, substituted or unsubstituted alkyl, substituted or unsubstituted cycloalkyl, substituted or unsubstituted alkenyl, substituted or unsubstituted alkynyl, substituted or unsubstituted aryl, substituted or unsubstituted heteroaryl, substituted or unsubstituted heterocycle;
   or L₃, X and L₄ taken together form a nitrogen containing heterocyclic ring;
   G is

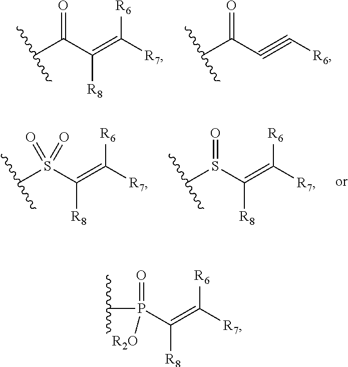

wherein,
   R₆, R₇ and R₈ are independently selected from among H, lower alkyl or substituted lower alkyl, lower heteroalkyl or substituted lower heteroalkyl, substituted or unsubstituted lower cycloalkyl, and substituted or unsubstituted lower heterocycloalkyl;
R₅ is H, halogen, -L₆-(substituted or unsubstituted C₁-C₃ alkyl), -L₆-(substituted or unsubstituted C₂-C₄ alkenyl), -L₆-(substituted or unsubstituted heteroaryl), or -L₆-(substituted or unsubstituted aryl), wherein L₆ is a bond, O, S, —S(═O), S(═O)₂, NH, C(O), —NHC(O)O, —OC(O)NH, —NHC(O), or —C(O)NH;
each R₉ is independently selected from among H, substituted or unsubstituted lower alkyl, and substituted or unsubstituted lower cycloalkyl;
each R₁₀ is independently H, substituted or unsubstituted lower alkyl, or substituted or unsubstituted lower cycloalkyl; or
two R₁₀ groups can together form a 5-, 6-, 7-, or 8-membered heterocyclic ring; or
R₁₀ and R₁₁ can together form a 5-, 6-, 7-, or 8-membered heterocyclic ring; or
each R₁₁ is independently selected from H or alkyl; and
   pharmaceutically active metabolites, pharmaceutically acceptable solvates,
   pharmaceutically acceptable salts, or pharmaceutically acceptable prodrugs thereof.
      In some embodiments, the compound of Formula (A) has the structure:

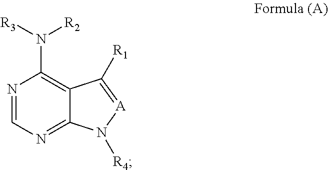

wherein:
A is N;
R₂ and R₃ are each H;
R₁ is phenyl-O-phenyl or phenyl-S-phenyl; and
R₄ is L₃-X-L₄-G, wherein,
   L₃ is optional, and when present is a bond, optionally substituted or unsubstituted alkyl, optionally substituted or unsubstituted cycloalkyl, optionally substituted or unsubstituted alkenyl, optionally substituted or unsubstituted alkynyl;
   X is optional, and when present is a bond, O, —C(═O), S, —S(═O), —S(═O)₂, —NH, —NR₉, —NHC(O), —C(O)NH, —NR₉C(O), —C(O)NR₉, —S(═O)₂NH, —NHS(═O)₂, —S(═O)₂NR₉—, —NR₉S(═O)₂, —OC(O)NH—, —NHC(O)O—, —OC(O)NR₉—, —NR₉C(O)O—, —CH═NO—, —ON═CH—, —NR₁₀C(O)NR₁₀—, heteroaryl, aryl, —NR₁₀C(═NR₁₁)NR₁₀—, —NR₁₀C(═NR₁₁)—, —C(═NR₁₁)NR₁₀—, —OC(═NR₁₁)—, or —C(═NR₁₁)O—;
   L₄ is optional, and when present is a bond, substituted or unsubstituted alkyl, substituted or unsubstituted cycloalkyl, substituted or unsubstituted alkenyl, substituted or unsubstituted alkynyl, substituted or unsubstituted aryl, substituted or unsubstituted heteroaryl, substituted or unsubstituted heterocycle;
   or L₃, X and L₄ taken together form a nitrogen containing heterocyclic ring;
   G is

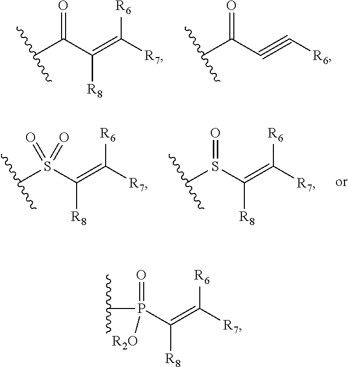

wherein,
R₆, R₇ and R₈ are independently selected from among H, lower alkyl or substituted lower alkyl, lower heteroalkyl or substituted lower heteroalkyl, substituted or unsubstituted lower cycloalkyl, and substituted or unsubstituted lower heterocycloalkyl.
   In some embodiments, the covalent BTK inhibitor is (R)-1-(3-(4-amino-3-(4-phenoxyphenyl)-1H-pyrazolo[3,4-d]pyrimidin-1-yl)piperidin-1-yl)prop-2-en-1-one

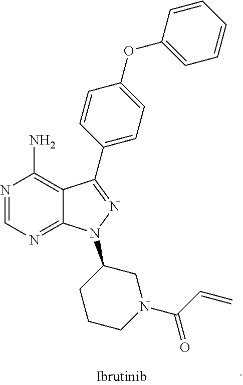 [SMILES: C=CC(=O)N1CCC[C@@H](n2nc(-c3ccc(Oc4ccccc4)cc3)c3c(N)ncnc32)C1]

In some embodiments, the anti-insulin B cells are mature anti-insulin B cells. In some embodiments, the population of mature anti-insulin B cells is reduced. In some embodiments, the method delays the onset of Type 1 Diabetes. In some embodiments, the method reduces the severity of Type 1 Diabetes. In some embodiments, the method prevents the development of Type 1 Diabetes. In some embodiments, the method further comprises administering an additional therapeutic agent. In some embodiments, the additional therapeutic agent is cyclosporine A. In some embodiments, the additional therapeutic agent is an anti-CD3 antibody. In some embodiments, the anti-CD3 antibody is teplizumab or otelixizumab.

Disclosed herein, in certain embodiments, are methods for decreasing the population of anti-insulin B cells in an individual in need thereof comprising administering to an individual in need thereof a composition comprising a therapeutically-effective amount of (R)-1-(3-(4-amino-3-(4-phenoxyphenyl)-1H-pyrazolo[3,4-d]pyrimidin-1-yl)piperidin-1-yl)prop-2-en-1-one

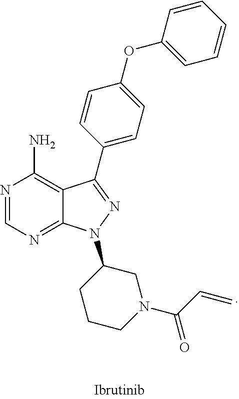 [SMILES: C=CC(=O)N1CCC[C@@H](n2nc(-c3ccc(Oc4ccccc4)cc3)c3c(N)ncnc32)C1]

In some embodiments, the anti-insulin B cells are mature anti-insulin B cells. In some embodiments, the population of mature anti-insulin B cells is reduced. In some embodiments, the method delays the onset of Type 1 Diabetes. In some embodiments, the method reduces the severity of Type 1 Diabetes. In some embodiments, the method prevents the development of Type 1 Diabetes. In some embodiments, the method further comprises administering an additional therapeutic agent. In some embodiments, the additional therapeutic agent is cyclosporine A. In some embodiments, the additional therapeutic agent is an anti-CD3 antibody. In some embodiments, the anti-CD3 antibody is teplizumab or otelixizumab.

### Certain Terminology

It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

As used herein, “amelioration” includes, but is not limited to, lessening of the severity of Type 1 Diabetes, delay of the onset of Type 1 Diabetes, or slowing of development of Type 1 Diabetes, whether permanent or temporary, lasting or transient, that can be attributed to or associated with administration of the compound or composition.

The term “Bruton's tyrosine kinase,” or BTK, as used herein, refers to Bruton's tyrosine kinase from Homo sapiens, as disclosed in, e.g., U.S. Pat. No. 6,326,469 (GenBank Accession No. NP_{—}000052).

The term “Bruton's tyrosine kinase homolog,” as used herein, refers to orthologs of Bruton's tyrosine kinase, e.g., the orthologs from mouse (GenBank Accession No. AAB47246), dog (GenBank Accession No. XP_{—}549139.), rat (GenBank Accession No. NP_{—}001007799), chicken (GenBank Accession No. NP_{—}989564), or zebra fish (GenBank Accession No. XP_{—}698117), and fusion proteins of any of the foregoing that exhibit kinase activity towards one or more substrates of Bruton's tyrosine kinase (e.g. a peptide substrate having the amino acid sequence “AVLESEEELYSSARQ”).

The term “homologous cysteine,” as used herein refers to a cysteine residue found within a sequence position that is homologous to that of cysteine 481 of Bruton's tyrosine kinase, as defined herein. For example, cysteine 482 is the homologous cysteine of the rat ortholog of Bruton's tyrosine kinase; cysteine 479 is the homologous cysteine of the chicken ortholog; and cysteine 481 is the homologous cysteine in the zebra fish ortholog. In another example, the homologous cysteine of TXK, a Tec kinase family member related to Bruton's tyrosine, is Cys 350.

The term “covalent Btk inhibitor”, as used herein, refers to an inhibitor that reacts with Btk to form a covalent complex. In some embodiments, the covalent Btk inhibitor is an irreversible Btk inhibitor.

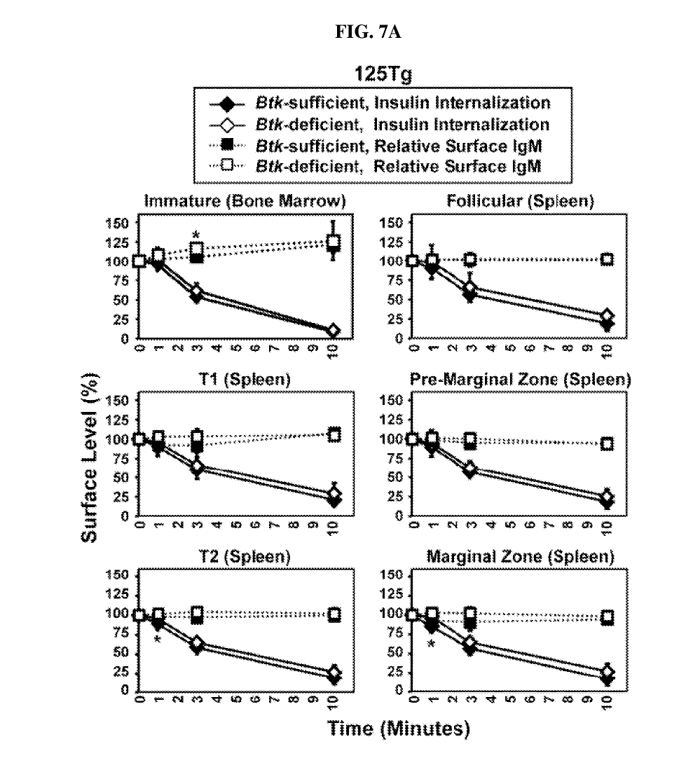

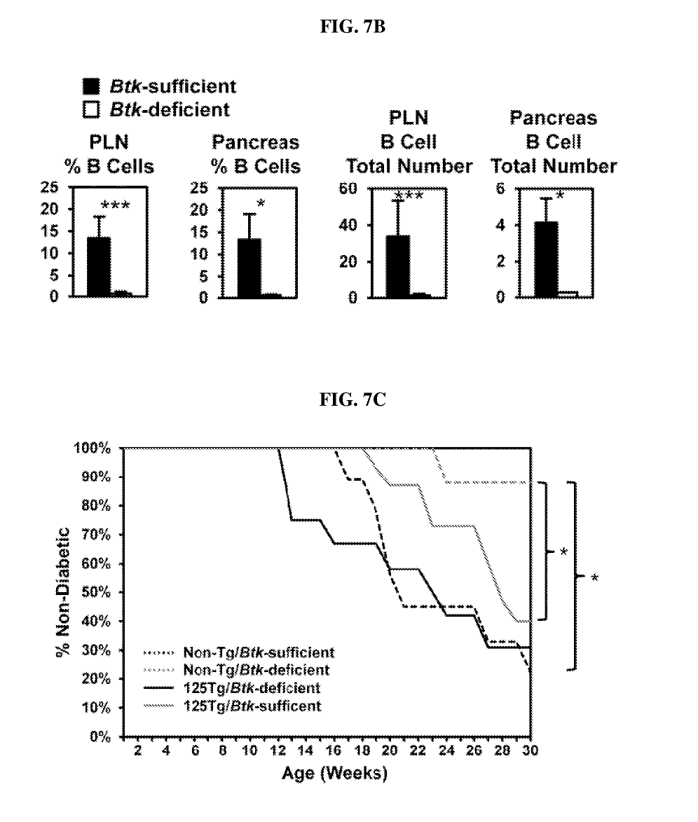

The term “irreversible Btk inhibitor,” as used herein, refers to an inhibitor of Btk that can form a covalent bond with an amino acid residue of Btk. In one embodiment, the irreversible inhibitor of Btk can form a covalent bond with a Cys residue of Btk; in particular embodiments, the irreversible inhibitor can form a covalent bond with a Cys 481 residue (or a homolog thereof) of Btk or a cysteine residue in the homologous corresponding position of another tyrosine kinase, as shown in FIG. 7.

The terms “individual”, “patient” and “subject” are used interchangeable. They refer to a mammal (e.g., a human) which is the object of treatment, or observation. The term is not to be construed as requiring the supervision of a medical practitioner (e.g., a physician, physician's assistant, nurse, orderly, hospice care worker).

The terms “treat,” “treating” or “treatment”, as used herein, include lessening the severity of Type 1 Diabetes, delaying the onset of Type 1 Diabetes, slowing the development of Type 1 Diabetes, causing regression/remission of Type 1 Diabetes, relieving a condition caused by Type 1 Diabetes, or stopping symptoms which result from Type 1 Diabetes. The terms “treat,” “treating” or “treatment”, include, but are not limited to, prophylactic and/or therapeutic treatments.

### Type 1 Diabetes

Described herein, in certain embodiments, are methods of preventing or delaying the onset of Type 1 Diabetes, inhibiting the maturation of anti-insulin B cells, or decreasing the population of anti-insulin B cells, in an individual in need thereof, comprising administering to the individual a composition comprising a therapeutically-effective amount of a Tec inhibitor. In some instances, the Tec inhibitor is a Btk inhibitor or an ITK inhibitor. In some instances, the Tec inhibitor is an ITK inhibitor. In some instances, the Tec inhibitor is a Btk inhibitor. In some cases, the Btk inhibitor is a covalent Btk inhibitor. In some embodiments, described herein include methods of preventing or delaying the onset of Type 1 Diabetes, or inhibiting the maturation of anti-insulin B cells, in an individual in need thereof, comprising administering to the individual a composition comprising a therapeutically-effective amount of a covalent Btk inhibitor. In some embodiments, a Btk inhibitor include PCI-45292, PCI-45466, AVL-101/CC-101 (Avila Therapeutics/Celgene Corporation), AVL-263/CC-263 (Avila Therapeutics/Celgene Corporation), AVL-292/CC-292 (Avila Therapeutics/Celgene Corporation), AVL-291/CC-291 (Avila Therapeutics/Celgene Corporation), CNX 774 (Avila Therapeutics), BMS-488516 (Bristol-Myers Squibb), BMS-509744 (Bristol-Myers Squibb), CGI-1746 (CGI Pharma/Gilead Sciences), CGI-560 (CGI Pharma/Gilead Sciences), CTA-056, GDC-0834 (Genentech), HY-11066 (also, CTK4I7891, HMS3265G21, HMS3265G22, HMS3265H21, HMS3265H22, 439574-61-5, AG-F-54930), ONO-4059 (Ono Pharmaceutical Co., Ltd.), ONO-WG37 (Ono Pharmaceutical Co., Ltd.), PLS-123 (Peking University), RN486 (Hoffmann-La Roche), HM71224 (Hanmi Pharmaceutical Company Limited), LFM-A13, BGB-3111 (Beigene), KBP-7536 (KBP BioSciences), ACP-196 (Acerta Pharma) and JTE-051 (Japan Tobacco Inc). In some embodiments, the covalent Btk inhibitor is an irreversible covalent Btk inhibitor. In some embodiments, the covalent Btk inhibitor is a compound of Formula A

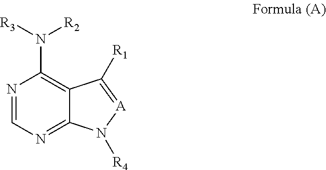

wherein
   A is independently selected from N or CR₅;
   R₁ is H, L₂-(substituted or unsubstituted alkyl), L₂-(substituted or unsubstituted cycloalkyl), L₂-(substituted or unsubstituted alkenyl), L₂-(substituted or unsubstituted cycloalkenyl), L₂-(substituted or unsubstituted heterocycle), L₂-(substituted or unsubstituted heteroaryl), or L₂-(substituted or unsubstituted aryl), where L₂ is a bond, O, S, —S(═O), —S(═O)₂, C(═O), -(substituted or unsubstituted C₁-C₆ alkyl), or -(substituted or unsubstituted C₂-C₆ alkenyl);
   R₂ and R₃ are independently selected from H, lower alkyl and substituted lower alkyl;
   R₄ is L₃-X-L₄-G, wherein,
      L₃ is optional, and when present is a bond, optionally substituted or unsubstituted alkyl, optionally substituted or unsubstituted cycloalkyl, optionally substituted or unsubstituted alkenyl, optionally substituted or unsubstituted alkynyl;
      X is optional, and when present is a bond, O, —C(═O), S, —S(═O), —S(═O)₂, —NH, —NR₉, —NHC(O), —C(O)NH, —NR₉C(O), —C(O)NR₉, —S(═O)₂NH, —NHS(═O)₂, —S(═O)₂NR₉—, —NR₉S(═O)₂, —OC(O)NH—, —NHC(O)O—, —OC(O)NR₉—, —NR₉C(O)O—, —CH═NO—, —ON═CH—, —NR₁₀C(O)NR₁₀—, heteroaryl, aryl, —NR₁₀C(═NR₁₁)NR₁₀—, —NR₁₀C(═NR₁₁)—, —C(═NR₁₁)NR₁₀—, —OC(═NR₁₁)—, or —C(═NR₁₁)O—;
      L₄ is optional, and when present is a bond, substituted or unsubstituted alkyl, substituted or unsubstituted cycloalkyl, substituted or unsubstituted alkenyl, substituted or unsubstituted alkynyl, substituted or unsubstituted aryl, substituted or unsubstituted heteroaryl, substituted or unsubstituted heterocycle;
      or L₃, X and L₄ taken together form a nitrogen containing heterocyclic ring;
      G is

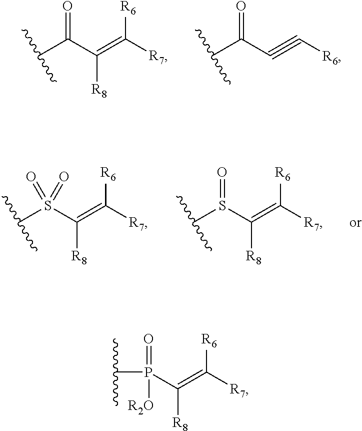

wherein,
   R₆, R₇ and R₈ are independently selected from among H, lower alkyl or substituted lower alkyl, lower heteroalkyl or substituted lower heteroalkyl, substituted or unsubstituted lower cycloalkyl, and substituted or unsubstituted lower heterocycloalkyl;
R₅ is H, halogen, -L₆-(substituted or unsubstituted C₁-C₃ alkyl), -L₆-(substituted or unsubstituted C₂-C₄ alkenyl), -L₆-(substituted or unsubstituted heteroaryl), or -L₆-(substituted or unsubstituted aryl), wherein L₆ is a bond, O, S, —S(═O), S(═O)₂, NH, C(O), —NHC(O)O, —OC(O)NH, —NHC(O), or —C(O)NH;
each R₉ is independently selected from among H, substituted or unsubstituted lower alkyl, and substituted or unsubstituted lower cycloalkyl;
each R₁₀ is independently H, substituted or unsubstituted lower alkyl, or substituted or unsubstituted lower cycloalkyl; or
two R₁₀ groups can together form a 5-, 6-, 7-, or 8-membered heterocyclic ring; or R₁₀ and R₁₁ can together form a 5-, 6-, 7-, or 8-membered heterocyclic ring; or
each R₁₁ is independently selected from H or alkyl; and
pharmaceutically active metabolites, pharmaceutically acceptable solvates, pharmaceutically acceptable salts, or pharmaceutically acceptable prodrugs thereof.
   In some embodiments, the covalent Btk inhibitor is ibrutinib.

Type I diabetes mellitus (T1D) is an autoimmune disease in which insulin-producing 13 cells in the islets of Langerhans of the pancreas are destroyed, resulting in hyperglycemia. Destruction of the islet cells results in loss of the ability to produce insulin. Insulin facilitates the movement of sugar from the bloodstream into cells, resulting in a decrease in the amount of sugar in the bloodstream. In T1D, the sugar builds up in the bloodstream, where it can cause life-threatening complications.

Physical symptoms of T1D include frequent urination, increased thirst, increased hunger, weight loss, fatigue, blurred vision, and numbness in the hands or feet. If blood sugar is extremely high, diabetic ketoacidosis may occur and related symptoms include rapid, deep breathing; dry skin and mouth; flushed face; fruity breath odor; nausea or vomiting; and stomach pain. Life-threatening long-term complications that can result from T1D including heart and blood vessel disease; neuropathy due to the injury of tiny blood vessel walls due to excess sugar; kidney damage (nephropathy); eye damage (diabetic retinopathy) which may lead to blindness; increased risk for cataracts and glaucoma; foot damage due to poor blood flow in the feel; skin and mouth conditions; osteoporosis; and pregnancy complications.

In some instances, T1D is further classified into three subtypes: autoimmune, nonautoimmune fulminant, and nonautoimmune nonfulminant (also known as nonautoimmune chronic) type 1 diabetes mellitus. Autoimmune type 1 diabetes mellitus comprises about 60% of the diabetic patient population and in some cases, is characterized by insulitis and over-expression of class I MHC molecules in the islet cells, a high prevalence of diabetes-related autoantibodies, and a progressive loss of beta-cell function after the onset of diabetes. Fulminant type 1 diabetes mellitus comprises about 10% of the patient population and in some cases, is characterized by a lack of insulitis or over-expression of class I HMC molecules, lymphocytic infiltration in the exocrine pancreatic tissue, elevated serum pancreatic enzyme levels, and absence of diabetes-related autoantibodies, and an aggressive disease course. Nonautoimmune nonfulminant type 1 diabetes mellitus comprises about 30% of the patient population, and in some cases, is characterized by a lack of insulitis or over-expression of class I MHC antigen in the islet cells, a low prevalence of diabetes-related autoantibodies, and a slow progression of beta-cell loss after onset of diabetes.

In some embodiments, described herein are methods of preventing or delaying the onset of a subtype of type 1 diabetes. In some embodiments, described herein are methods of preventing or delaying the onset of autoimmune type 1 diabetes mellitus. In some embodiments, described herein are methods of preventing or delaying the onset of fulminant type 1 diabetes mellitus. In some embodiments, described herein are methods of preventing or delaying the onset of nonautoimmune nonfulminant type 1 diabetes mellitus.

In some embodiments, further described herein are methods of inhibiting the maturation of anti-insulin B cells in a subtype of type 1 diabetes. In some embodiments, described herein methods of inhibiting the maturation of anti-insulin B cells in autoimmune type 1 diabetes mellitus. In some embodiments, described herein methods of inhibiting the maturation of anti-insulin B cells in fulminant type 1 diabetes mellitus. In some embodiments, described herein methods of inhibiting the maturation of anti-insulin B cells in nonautoimmune nonfulminant type 1 diabetes mellitus.

In some instances, additionally described herein are methods of decreasing the population of anti-insulin B cells in a subtype of type 1 diabetes. In some embodiments, described herein methods of decreasing the population of anti-insulin B cells in autoimmune type 1 diabetes mellitus. In some embodiments, described herein methods of decreasing the population of anti-insulin B cells in fulminant type 1 diabetes mellitus. In some embodiments, described herein methods of decreasing the population of anti-insulin B cells in nonautoimmune nonfulminant type 1 diabetes mellitus.

T1D differs from type 2 diabetes because in type 2 diabetes, the islet cells are still functioning, but the body becomes resistant to insulin, or the pancreas doesn't produce enough insulin or both.

Further, Type 1 diabetes can be distinguished from type 2 diabetes due to the presence of autoantibodies to insulin, 65 kDa form of glutamic acid decarboxylase (GAD65), insulinoma antigen 2 (IA-2), and zinc transporter 8 (ZnT8). Expression of one or more of the autoantibodies is a risk factor for development of T1D. Expression of at least two of the four autoantibodies is a significant risk factor for development of T1D.

B cells play an important role in the adaptive immune system. Upon infection, activated B cells are differentiated into plasma cells; this results in the production of antigen specific antibodies. The development of B cells occurs in two stages. In the first stage, the B cell egresses from the bone marrow and IgD and IgM appear on the B cell surface. This development is independent of exogenous antigens. In the second stage, the B cell encounters an antigen and is activated. Antigens can stimulate B cells through BCR crosslinking, expression of other interacting surface molecules or cytokine expression.

Autoreactive B cells play a pathogenic role in T1D by acting as antigen-presenting cells and autoantibody secretors, which lead to T cell-mediated (e.g. CD8+ T cell-mediated) autoimmune destruction of pancreatic B cells. For example, most autoreactive B cells are culled during development in the bone marrow, but a few of the autoreactive B cells can escape this process and emerge into the periphery in an anergic state, a state in which the B cells does not proliferate or produce antibody. However, in some instances these B cells retain the ability to present antigen to autoreactive T cells, a mechanism which support T1D. In humans, about 2.5% of B cells emergy into an anergic, autoreactive-prone phenotype.

In some instances, anti-insulin B cells are anergic autoreactive B cells. In some embodiments, the anti-insulin B cells are mature anti-insulin B cells. In some instances, anti-insulin B cells mediate the development of T1D. In some instances, mature anti-insulin B cells mediate the develoment of T1D.

Bruton's tyrosine kinase (BTK) is a cytosolic Tec kinase family protein that participates in signal propagation from the BCR, resulting in downstream nuclear translocation of transcription factors NF-AT and NF-κB, which leads to modulation of B cell maturation and function. In one study, BTK depletion has been shown to influence the survival of autoreactive B cells, e.g. anti-insulin B cells (Bonami, et al. “Bruton's tyrosine kinase promotes persistence of mature anti-insulin B cells,” J. Immunol 192:1459-1470 (2014)). Indeed, the study showed that genetic targeting of BTK eliminated about 95% of mature anti-insulin B cells, in contrast to about 18% loss of non anti-insulin B cells.

Individuals are considered to be prediabetic if they demonstrate impaired fasting glycaemia and/or impaired glucose tolerance Impaired fasting glycaemia is found where fasting blood glucose is elevated above what is considered normal levels but is not high enough to be classified as diabetes mellitus. Individuals with impaired fasting glycaemia have a 50% risk over 10 years of progressing to overt diabetes Impaired fasting glycaemia is found wherein fasting plasma glucose is between about 6.1 mmol/l (110 mg/dL) to about 6.9 mmol/L (125 mg/dL) or between about 5.6 mmol/L (100 mg/dL) to about 6.9 mmol/L (125 mg/dL).

Impaired glucose tolerance is associated with insulin resistance and increased risk of cardiovascular pathology. According to the criteria of the World Health Organization and the American Diabetes Association, criteria for impaired glucose tolerance are two-hour glucose levels of 140 to 199 mg per dL (7.8 to 11.0 mmol) on the 75-g oral glucose tolerance test.

Additional criteria for the diagnosis of T1D include measurement of the A1C level. A1C, also known as glycated hemoglobin, glycosylated hemoglobin, hemoglobin A1C, or HbA1c, test the blood sugar levels over the span of two to three months. A1C measures the percentage of glycated hemoglobins, or hemoglobin that is coated with sugar. A normal A1C level is from about 4.5 to about 6%. A level of about 6.5% and higher indicates the presence of diabetes in an individual. In some instances, A1C level correlates to average blood sugar level. Table 1 illustrates A1C level and its corresponding estimated average blood sugar level.

TABLE 1

| | Estimated average |
|---|---|
| A1C level | blood sugar level |
| 5 percent | 97 mg/dL (5.4 mmol/L) |
| 6 percent | 126 mg/dL (7 mmol/L) |
| 7 percent | 154 mg/dL (8.5 mmol/L) |
| 8 percent | 183 mg/dL (10.2 mmol/L) |
| 9 percent | 212 mg/dL (11.8 mmol/L) |
| 10 percent | 240 mg/dL (13.3 mmol/L) |
| 11 percent | 269 mg/dL (14.9 mmol/L) |
| 12 percent | 298 mg/dL (16.5 mmol/L) |
| 13 percent | 326 mg/dL (18.1 mmol/L) |
| 14 percent | 355 mg/dL (19.7 mmol/L) |

### Combination Therapies

Described herein, in certain embodiments, are methods of preventing or delaying the onset of Type 1 Diabetes, inhibiting the maturation of anti-insulin B cells, or decreasing the population of anti-insulin B cells, in an individual in need thereof, comprising co-administering to the individual a Tec inhibitor and an additional therapeutic agent. In some embodiments, the Tec inhibitor is a Btk inhibitor or an ITK inhibitor. In some embodiments, the Tec inhibitor is an ITK inhibitor. In some embodiments, the Tec inhibitor is a Btk inhibitor. In some embodiments, the Btk inhibitor include PCI-45292, PCI-45466, AVL-101/CC-101 (Avila Therapeutics/Celgene Corporation), AVL-263/CC-263 (Avila Therapeutics/Celgene Corporation), AVL-292/CC-292 (Avila Therapeutics/Celgene Corporation), AVL-291/CC-291 (Avila Therapeutics/Celgene Corporation), CNX 774 (Avila Therapeutics), BMS-488516 (Bristol-Myers Squibb), BMS-509744 (Bristol-Myers Squibb), CGI-1746 (CGI Pharma/Gilead Sciences), CGI-560 (CGI Pharma/Gilead Sciences), CTA-056, GDC-0834 (Genentech), HY-11066 (also, CTK4I7891, HMS3265G21, HMS3265G22, HMS3265H21, HMS3265H22, 439574-61-5, AG-F-54930), ONO-4059 (Ono Pharmaceutical Co., Ltd.), ONO-WG37 (Ono Pharmaceutical Co., Ltd.), PLS-123 (Peking University), RN486 (Hoffmann-La Roche), HM71224 (Hanmi Pharmaceutical Company Limited), LFM-A13, BGB-3111 (Beigene), KBP-7536 (KBP BioSciences), ACP-196 (Acerta Pharma) and JTE-051 (Japan Tobacco Inc). In some embodiments, the Btk inhibitor is a covalent Btk inhibitor. In some embodiments, described herein include methods of preventing or delaying the onset of Type 1 Diabetes, inhibiting the maturation of anti-insulin B cells, or decreasing the population of B cells, in an individual in need thereof, comprising co-administering to the individual (a) a therapeutically-effective amount of a covalent Btk inhibitor, and (b) a therapeutically-effective amount of an additional therapeutic agent. In some embodiments, the covalent Btk inhibitor is an irreversible covalent Btk inhibitor. In some embodiments, the covalent Btk inhibitor is a compound of Formula A

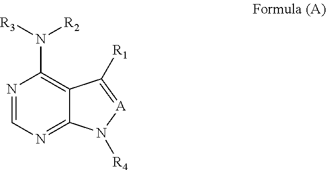

wherein
   A is independently selected from N or CR₅;
   R₁ is H, L₂-(substituted or unsubstituted alkyl), L₂-(substituted or unsubstituted cycloalkyl), L₂-(substituted or unsubstituted alkenyl), L₂-(substituted or unsubstituted cycloalkenyl), L₂-(substituted or unsubstituted heterocycle), L₂-(substituted or unsubstituted heteroaryl), or L₂-(substituted or unsubstituted aryl), where L₂ is a bond, O, S, —S(═O), —S(═O)₂, C(═O), -(substituted or unsubstituted C₁-C₆ alkyl), or -(substituted or unsubstituted C₂-C₆ alkenyl);
   R₂ and R₃ are independently selected from H, lower alkyl and substituted lower alkyl;
   R₄ is L₃-X-L₄-G, wherein,
      L₃ is optional, and when present is a bond, optionally substituted or unsubstituted alkyl, optionally substituted or unsubstituted cycloalkyl, optionally substituted or unsubstituted alkenyl, optionally substituted or unsubstituted alkynyl;
      X is optional, and when present is a bond, O, —C(═O), S, —S(═O), —S(═O)₂, —NH, —NR₉, —NHC(O), —C(O)NH, —NR₉C(O), —C(O)NR₉, —S(═O)₂NH, —NHS(═O)₂, —S(═O)₂NR₉—, —NR₉S(═O)₂, —OC(O)NH—, —NHC(O)O—, —OC(O)NR₉—, —NR₉C(O)O—, —CH═NO—, —ON═CH—, —NR₁₀C(O)NR₁₀—, heteroaryl, aryl, —NR₁₀C(═NR₁₁)NR₁₀—, —NR₁₀C(═NR₁₁)—, —C(═NR₁₁)NR₁₀—, —OC(═NR₁₁)—, or —C(═NR₁₁)O—;
      L₄ is optional, and when present is a bond, substituted or unsubstituted alkyl, substituted or unsubstituted cycloalkyl, substituted or unsubstituted alkenyl, substituted or unsubstituted alkynyl, substituted or unsubstituted aryl, substituted or unsubstituted heteroaryl, substituted or unsubstituted heterocycle;
      or L₃, X and L₄ taken together form a nitrogen containing heterocyclic ring;
      G is

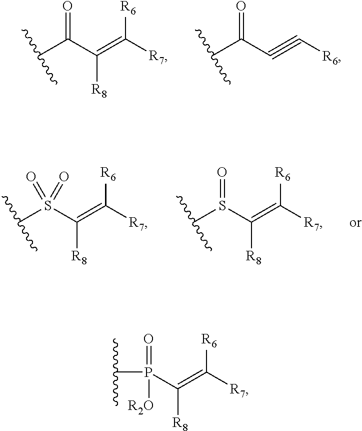

wherein,
   R₆, R₇ and R₈ are independently selected from among H, lower alkyl or substituted lower alkyl, lower heteroalkyl or substituted lower heteroalkyl, substituted or unsubstituted lower cycloalkyl, and substituted or unsubstituted lower heterocycloalkyl;
R₅ is H, halogen, -L₆-(substituted or unsubstituted C₁-C₃ alkyl), -L₆-(substituted or unsubstituted C₂-C₄ alkenyl), -L₆-(substituted or unsubstituted heteroaryl), or -L₆-(substituted or unsubstituted aryl), wherein L₆ is a bond, O, S, —S(═O), S(═O)₂, NH, C(O), —NHC(O)O, —OC(O)NH, —NHC(O), or —C(O)NH;
each R₉ is independently selected from among H, substituted or unsubstituted lower alkyl, and substituted or unsubstituted lower cycloalkyl;
each R₁₀ is independently H, substituted or unsubstituted lower alkyl, or substituted or unsubstituted lower cycloalkyl; or
two R₁₀ groups can together form a 5-, 6-, 7-, or 8-membered heterocyclic ring; or
R₁₀ and R₁₁ can together form a 5-, 6-, 7-, or 8-membered heterocyclic ring; or
each R₁₁ is independently selected from H or alkyl; and
pharmaceutically active metabolites, pharmaceutically acceptable solvates, pharmaceutically acceptable salts, or pharmaceutically acceptable prodrugs thereof. In some embodiments, the covalent Btk inhibitor is ibrutinib.

In some embodiments, the additional therapeutic agent is a Type 1 Diabetes therapeutic agent.

In some embodiments, the additional therapeutic agent is cyclosporine A.

In some embodiments, the additional therapeutic agent is an anti-CD3 antibody. In some embodiments, the anti-CD3 antibody is teplizumab, otelixizumab, or visilizumab. In some instances, the administration of anti-CD3 antibody induces T cell activation. In some instances, the administration of anti-CD3 antibody modulates T cell response. In some instances, the administration of anti-CD3 antibody modulates Th1 cell response, a Th2 cell response, or a combination thereof. In some instances, the administration of anti-CD3 antibody modulates Th1 cell response. In some instances, the administration of anti-CD3 antibody inhibits Th1 cell response, Th2 cell response, or a combination thereof. In some instances, the administration of anti-CD3 antibody inhibits Th1 cell response.

In some embodiments, the additional therapeutic agent is an anti-CD20 antibody. In some embodiments, the anti-CD20 antibody is rituximab, ocrelizumab, ofatumumab. In some embodiments, the additional therapeutic agent is belimumab or atacicept.

In some embodiments, the additional therapeutic agent is insulin. In some embodiments, the additional therapeutic agent is pramlintide acetate. In some embodiments, the additional therapeutic agent is sitagliptin. In some embodiments, the additional therapeutic agent is a proton-pump inhibitor. In some embodiments, the proton-pump inhibitor is lansoprazole. In some embodiments, the additional therapeutic agent is a thiazolidinedione. In some embodiments, the thiazolidinedione is pioglitazone or rosiglitazone. In some embodiments, the additional therapeutic agent is aldesleukin.

In some embodiments, the additional therapeutic agent is a serine protease. In some embodiments, the serine protease is a tissue kallikrein-1 protein. In some embodiments, the tissue kallikrein-1 protein is a human tissue kallikrein-1 protein (KLK1). In some embodiments, the additional therapeutic agent is a human KLK1 as described in Maneva-Radicheva et al. PLoS One, 2014, 9:e107213.

In some embodiments, the additional therapeutic agent is a stem cell. In some instances, the stem cell is an induced pluripotent stem cell (iPS), embryonic stem cell (ESC), or a tissue-derived-stem cell such as a bone marrow-, adipose-, or cord blood-derived-stem cell.

In some embodiments, the additional therapeutic agent is a diabetogenic peptide. In some embodiments, the diabetogenic peptide is P277. In some embodiments, P277 is fused to a heat shock protein, HSP65. In some embodiments, the additional therapeutic agent is a HSP65-P277 fusion protein.

In some embodiments, the additional therapeutic agent is a plant-derived compound. In some embodiments, the plant-derived compound is a flavonol, such as epicatechin. In some embodiments, the additional therapeutic agent is epicatechin (Fu et al., J. Agric Food Chem 2013, 61:4303-4309).

When an additional agent is co-administered with a covalent Btk inhibitor (e.g., an irreversible covalent Btk inhibitor, e.g., ibrutinib), the additional agent and the covalent Btk inhibitor (e.g., an irreversible covalent Btk inhibitor, e.g., ibrutinib) do not have to be administered in the same pharmaceutical composition, and are optionally, because of different physical and chemical characteristics, administered by different routes. The initial administration is made, for example, according to established protocols, and then, based upon the observed effects, the dosage, modes of administration and times of administration are modified.

Or, by way of example only, the therapeutic effectiveness of a covalent Btk inhibitor (e.g., an irreversible covalent Btk inhibitor, e.g., ibrutinib) described herein is enhanced by administration of an adjuvant (i.e., by itself the adjuvant has minimal therapeutic benefit, but in combination with another therapeutic agent, the overall therapeutic benefit to the patient is enhanced). Or, by way of example only, the benefit experienced by an individual is increased by administering a covalent Btk inhibitor (e.g., an irreversible covalent Btk inhibitor, e.g., ibrutinib) described herein with another therapeutic agent (which also includes a therapeutic regimen) that also has therapeutic benefit. In any case, regardless of the disease, disorder being treated, the overall benefit experienced by the patient is in some embodiments simply additive of the two therapeutic agents or in other embodiments, the patient experiences a synergistic benefit.

The particular choice of compounds used will depend upon the diagnosis of the attending physicians and their judgment of the condition of the patient and the appropriate treatment protocol. The compounds are optionally administered concurrently (e.g., simultaneously, essentially simultaneously or within the same treatment protocol) or sequentially, depending upon the nature of the disorder, the condition of the patient, and the actual choice of compounds used. The determination of the order of administration, and the number of repetitions of administration of each therapeutic agent during a treatment protocol, is based on an evaluation of the disease being treated and the condition of the patient.

In some embodiments, therapeutically-effective dosages vary when the drugs are used in treatment combinations. Methods for experimentally determining therapeutically-effective dosages of drugs and other agents for use in combination treatment regimens are described in the literature. For example, the use of metronomic dosing, i.e., providing more frequent, lower doses in order to minimize toxic side effects, has been described extensively in the literature Combination treatment further includes periodic treatments that start and stop at various times to assist with the clinical management of the patient.

For combination therapies described herein, dosages of the co-administered compounds will of course vary depending on the type of co-drug employed, on the specific drug employed, on the disorder being treated and so forth. In addition, when co-administered with an additional therapeutic agent, a covalent Btk inhibitor (e.g., an irreversible covalent Btk inhibitor, e.g., ibrutinib) described herein is administered either simultaneously with the additional therapeutic agent, or sequentially. If administered sequentially, the attending physician will decide on the appropriate sequence of administering protein in combination with the biologically active agent(s).

If the additional therapeutic agent and the covalent Btk inhibitor (e.g., an irreversible covalent Btk inhibitor, e.g., ibrutinib) are administered simultaneously, the multiple therapeutic agents are optionally provided in a single, unified form, or in multiple forms (by way of example only, either as a single pill or as two separate pills). In some embodiments, one of the therapeutic agents is given in multiple doses, or both are given as multiple doses. If not simultaneous, the timing between the multiple doses is from about more than zero weeks to less than about four weeks. In addition, the combination methods, compositions and formulations are not to be limited to the use of only two agents; the use of multiple therapeutic combinations is also envisioned.

It is understood that the dosage regimen to treat, prevent, or ameliorate the condition(s) for which relief is sought, can be modified in accordance with a variety of factors. These factors include the disorder from which the subject suffers, as well as the age, weight, sex, diet, and medical condition of the subject. Thus, the dosage regimen actually employed can vary widely and therefore can deviate from the dosage regimens set forth herein.

In some embodiments, the pharmaceutical agents which make up the combination therapy disclosed herein are administered in a combined dosage form, or in separate dosage forms intended for substantially simultaneous administration. In some embodiments, the pharmaceutical agents that make up the combination therapy are administered sequentially, with either therapeutic compound being administered by a regimen calling for two-step administration. In some embodiments, the two-step administration regimen calls for sequential administration of the active agents or spaced-apart administration of the separate active agents. The time period between the multiple administration steps ranges from a few minutes to several hours, depending upon the properties of each pharmaceutical agent, such as potency, solubility, bioavailability, plasma half-life and kinetic profile of the pharmaceutical agent. In some embodiments, circadian variation of the target molecule concentration determines the optimal dose interval.

In some embodiments, the covalent Btk inhibitor (e.g., an irreversible covalent Btk inhibitor, e.g., ibrutinib) and the additional therapeutic agent are administered in a unified dosage form. In some embodiments, the covalent Btk inhibitor (e.g., an irreversible covalent Btk inhibitor, e.g., ibrutinib) and the additional therapeutic agent are administered in separate dosage forms. In some embodiments, the covalent Btk inhibitor (e.g., an irreversible covalent Btk inhibitor, e.g., ibrutinib) and the additional therapeutic agent are administered simultaneously or sequentially.

### Administration

Described herein methods of preventing or delaying the onset of Type 1 Diabetes, inhibiting the maturation of anti-insulin B cells, or decreasing the population of anti-insulin B cells in an individual in need thereof comprising administering to the individual a composition comprising a therapeutically-effective amount of a Tec inhibitor. In some instances, the Tec inhibitor is a Btk inhibitor or an ITK inhibitor. In some instances, the Tec inhibitor is an ITK inhibitor. In some instances, the Tec inhibitor is a Btk inhibitor. In some instances, the Btk inhibitor include PCI-45292, PCI-45466, AVL-101/CC-101 (Avila Therapeutics/Celgene Corporation), AVL-263/CC-263 (Avila Therapeutics/Celgene Corporation), AVL-292/CC-292 (Avila Therapeutics/Celgene Corporation), AVL-291/CC-291 (Avila Therapeutics/Celgene Corporation), CNX 774 (Avila Therapeutics), BMS-488516 (Bristol-Myers Squibb), BMS-509744 (Bristol-Myers Squibb), CGI-1746 (CGI Pharma/Gilead Sciences), CGI-560 (CGI Pharma/Gilead Sciences), CTA-056, GDC-0834 (Genentech), HY-11066 (also, CTK4I7891, HMS3265G21, HMS3265G22, HMS3265H21, HMS3265H22, 439574-61-5, AG-F-54930), ONO-4059 (Ono Pharmaceutical Co., Ltd.), ONO-WG37 (Ono Pharmaceutical Co., Ltd.), PLS-123 (Peking University), RN486 (Hoffmann-La Roche), HM71224 (Hanmi Pharmaceutical Company Limited), LFM-A13, BGB-3111 (Beigene), KBP-7536 (KBP BioSciences), ACP-196 (Acerta Pharma) and JTE-051 (Japan Tobacco Inc). In some instances, the Btk inhibitor is a covalent Btk inhibitor. In some embodiments, described herein methods of preventing or delaying the onset of Type 1 Diabetes, inhibiting the maturation of anti-insulin B cells, or decreasing the population of anti-insulin B cells in an individual in need thereof comprising administering to the individual a composition comprising a therapeutically-effective amount of a covalent Btk inhibitor (e.g., an irreversible covalent Btk inhibitor, e.g., ibrutinib). In some embodiments, the covalent Btk inhibitor is (R)-1-(3-(4-amino-3-(4-phenoxyphenyl)-1H-pyrazolo[3,4-d]pyrimidin-1-yl)piperidin-1-yl)prop-2-en-1-one (i.e. PCI-32765/ibrutinib).

The covalent Btk inhibitor (e.g., an irreversible covalent Btk inhibitor, e.g., ibrutinib) is administered before, during or after the development of Type 1 Diabetes. In some embodiments, the covalent Btk inhibitor (e.g., an irreversible covalent Btk inhibitor, e.g., ibrutinib) is used as a prophylactic and is administered continuously to subjects with a propensity to develop Type 1 Diabetes. In some embodiments, the covalent Btk inhibitor (e.g., an irreversible covalent Btk inhibitor, e.g., ibrutinib) is administered to an individual during or as soon as possible after the development of Type 1 Diabetes.

In some embodiments, the initial administration of the covalent Btk inhibitor (e.g., an irreversible covalent Btk inhibitor, e.g., ibrutinib) is via any route practical, such as, for example, an intravenous injection, a bolus injection, infusion over 5 minutes to about 5 hours, a pill, a capsule, transdermal patch, buccal delivery, and the like, or combination thereof.

The covalent Btk inhibitor (e.g., an irreversible covalent Btk inhibitor, e.g., ibrutinib) should be administered as soon as is practicable after the onset of a disorder is detected or suspected, and for a length of time necessary for the treatment of the disease, such as, for example, from about 1 month to about 3 months, or continuously throughout the individual's life. The length of treatment can vary for each subject, and the length can be determined using the known criteria. In some embodiments, the covalent Btk inhibitor (e.g., an irreversible covalent Btk inhibitor, e.g., ibrutinib) is administered for at least 2 weeks, between about 1 month to about 5 years, or from about 1 month to about 3 years. In some embodiments, the covalent Btk inhibitor (e.g., an irreversible covalent Btk inhibitor, e.g., ibrutinib) is administered throughout the individual's life.

Therapeutically effective amounts will depend on the severity and course of the disorder, previous therapy, the patient's health status, weight, and response to the drugs, and the judgment of the treating physician. Prophylactically effective amounts depend on the patient's state of health, weight, the severity and course of the disease, previous therapy, response to the drugs, and the judgment of the treating physician.

In some embodiments, the covalent Btk inhibitor (e.g., an irreversible covalent Btk inhibitor, e.g., ibrutinib) is administered to the patient on a regular basis, e.g., three times a day, two times a day, once a day, every other day or every 3 days. In other embodiments, the covalent Btk inhibitor (e.g., an irreversible covalent Btk inhibitor, e.g., ibrutinib) is administered to the patient on an intermittent basis, e.g., twice a day followed by once a day followed by three times a day; or the first two days of every week; or the first, second and third day of a week. In some embodiments, intermittent dosing is as effective as regular dosing. In further or alternative embodiments, the covalent Btk inhibitor (e.g., an irreversible covalent Btk inhibitor, e.g., ibrutinib) is administered only when the patient exhibits a particular symptom, e.g., the onset of pain, or the onset of a fever, or the onset of an inflammation, or the onset of a skin disorder. Dosing schedules of each compound may depend on the other or may be independent of the other.

In the case wherein the patient's condition does not improve, upon the doctor's discretion the administration of the compounds may be administered chronically, that is, for an extended period of time, including throughout the duration of the patient's life in order to ameliorate or otherwise control or limit the symptoms of the patient's disorder.

In the case wherein the patient's status does improve, upon the doctor's discretion the administration of the compounds may be given continuously; alternatively, the dose of drug being administered may be temporarily reduced or temporarily suspended for a certain length of time (i.e., a “drug holiday”). The length of the drug holiday can vary between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday may be from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance regimen is administered if necessary. Subsequently, the dosage or the frequency of administration, or both, of the covalent Btk inhibitor (e.g., an irreversible covalent Btk inhibitor, e.g., ibrutinib) can be reduced, as a function of the symptoms, to a level at which the individual's improved condition is retained. Individuals can, however, require intermittent treatment on a long-term basis upon any recurrence of symptoms.

The amount of the covalent Btk inhibitor (e.g., an irreversible covalent Btk inhibitor, e.g., ibrutinib) will vary depending upon factors such as the particular compound, disorder and its severity, the identity (e.g., weight) of the subject or host in need of treatment, and is determined according to the particular circumstances surrounding the case, including, e.g., the specific agents being administered, the routes of administration, and the subject or host being treated. In general, however, doses employed for adult human treatment will typically be in the range of 0.02-5000 mg per day, or from about 1-3000 mg per day. The desired dose may be presented in a single dose or as divided doses administered simultaneously (or over a short period of time) or at appropriate intervals, for example as two, three, four or more sub-doses per day.

In some embodiments, the therapeutic amount of the covalent Btk inhibitor (e.g., an irreversible covalent Btk inhibitor, e.g., ibrutinib) is from 100 mg/day up to, and including, 2000 mg/day. In some embodiments, the amount of the covalent Btk inhibitor (e.g., an irreversible covalent Btk inhibitor, e.g., ibrutinib) is from 140 mg/day up to, and including, 840 mg/day. In some embodiments, the amount of the covalent Btk inhibitor (e.g., an irreversible covalent Btk inhibitor, e.g., ibrutinib) is from 420 mg/day up to, and including, 840 mg/day. In some embodiments, the amount of the covalent Btk inhibitor (e.g., an irreversible covalent Btk inhibitor, e.g., ibrutinib) is about 140 mg/day. In some embodiments, the amount of the covalent Btk inhibitor (e.g., an irreversible covalent Btk inhibitor, e.g., ibrutinib) is about 280 mg/day. In some embodiments, the amount of the covalent Btk inhibitor (e.g., an irreversible covalent Btk inhibitor, e.g., ibrutinib) is about 420 mg/day. In some embodiments, the amount of the covalent Btk inhibitor (e.g., an irreversible covalent Btk inhibitor, e.g., ibrutinib) is about 560 mg/day. In some embodiments, the amount of the covalent Btk inhibitor (e.g., an irreversible covalent Btk inhibitor, e.g., ibrutinib) is about 700 mg/day. In some embodiments, the amount of the covalent Btk inhibitor (e.g., an irreversible covalent Btk inhibitor, e.g., ibrutinib) is about 840 mg/day. In some embodiments, the amount of the covalent Btk inhibitor (e.g., an irreversible covalent Btk inhibitor, e.g., ibrutinib) is about 980 mg/day. In some embodiments, the amount of the covalent Btk inhibitor (e.g., an irreversible covalent Btk inhibitor, e.g., ibrutinib) is about 1120 mg/day. In some embodiments, the amount of the covalent Btk inhibitor (e.g., an irreversible covalent Btk inhibitor, e.g., ibrutinib) is about 1260 mg/day. In some embodiments, the amount of the covalent Btk inhibitor (e.g., an irreversible covalent Btk inhibitor, e.g., ibrutinib) is about 140 mg/day.

In some embodiments, the dosage of the covalent Btk inhibitor (e.g., an irreversible covalent Btk inhibitor, e.g., ibrutinib) is escalated over time. In some embodiments, the dosage of the covalent Btk inhibitor (e.g., an irreversible covalent Btk inhibitor, e.g., ibrutinib) is escalated from at or about 1.25 mg/kg/day to at or about 12.5 mg/kg/day over a predetermined period of time. In some embodiments the predetermined period of time is over 1 month, over 2 months, over 3 months, over 4 months, over 5 months, over 6 months, over 7 months, over 8 months, over 9 months, over 10 months, over 11 months, over 12 months, over 18 months, over 24 months or longer.

The covalent Btk inhibitor (e.g., an irreversible covalent Btk inhibitor, e.g., ibrutinib) may be formulated into unit dosage forms suitable for single administration of precise dosages. In unit dosage form, the formulation is divided into unit doses containing appropriate quantities of one or both compounds. The unit dosage may be in the form of a package containing discrete quantities of the formulation. Non-limiting examples are packaged tablets or capsules, and powders in vials or ampoules. Aqueous suspension compositions can be packaged in single-dose non-reclosable containers. Alternatively, multiple-dose reclosable containers can be used, in which case it is typical to include a preservative in the composition. By way of example only, formulations for parenteral injection may be presented in unit dosage form, which include, but are not limited to ampoules, or in multi-dose containers, with an added preservative.

It is understood that a medical professional will determine the dosage regimen in accordance with a variety of factors. These factors include the solid tumor from which the subject suffers, the degree of metastasis, as well as the age, weight, sex, diet, and medical condition of the subject.

### Compounds

Described herein methods of preventing or delaying the onset of Type 1 Diabetes, inhibiting the maturation of anti-insulin B cells, or decreasing the population of anti-insulin B cells, in an individual in need thereof comprising administering to the individual a composition comprising a therapeutically-effective amount of a TEC inhibitor (e.g., BTK inhibitor or ITK inhibitor). In some instances, the BTK inhibitor is a covalent Btk inhibitor. In some embodiments, described herein are methods of preventing or delaying the onset of Type 1 Diabetes, inhibiting the maturation of anti-insulin B cells, or decreasing the population of anti-insulin B cells, in an individual in need thereof comprising administering to the individual a composition comprising a therapeutically-effective amount of a covalent Btk inhibitor (e.g., an irreversible covalent Btk inhibitor, e.g., ibrutinib).

Definition of standard chemistry terms are found in reference works, including Carey and Sundberg “ADVANCED ORGANIC CHEMISTRY 4^{TH} ED.” Vols. A (2000) and B (2001), Plenum Press, New York. Unless otherwise indicated, conventional methods of mass spectroscopy, NMR, HPLC, protein chemistry, biochemistry, recombinant DNA techniques and pharmacology, within the skill of the art are employed. Unless specific definitions are provided, the nomenclature employed in connection with, and the laboratory procedures and techniques of, analytical chemistry, synthetic organic chemistry, and medicinal and pharmaceutical chemistry described herein are those known in the art. Standard techniques are optionally used for chemical syntheses, chemical analyses, pharmaceutical preparation, formulation, and delivery, and treatment of patients. Standard techniques are optionally used for recombinant DNA, oligonucleotide synthesis, and tissue culture and transformation (e.g., electroporation, lipofection). Reactions and purification techniques are performed using documented methodologies or as described herein.

It is to be understood that the methods and compositions described herein are not limited to the particular methodology, protocols, cell lines, constructs, and reagents described herein and as such optionally vary. It is also to be understood that the terminology used herein is for the purpose of describing particular embodiments only, and is not intended to limit the scope of the methods and compositions described herein, which will be limited only by the appended claims.

Unless stated otherwise, the terms used for complex moieties (i.e., multiple chains of moieties) are to be read equivalently either from left to right or right to left. For example, the group alkylenecycloalkylene refers both to an alkylene group followed by a cycloalkylene group or as a cycloalkylene group followed by an alkylene group.

The suffix “one” appended to a group indicates that such a group is a diradical. By way of example only, a methylene is a diradical of a methyl group, that is, it is a —CH₂— group; and an ethylene is a diradical of an ethyl group, i.e., —CH₂CH₂—.

An “alkyl” group refers to an aliphatic hydrocarbon group. The alkyl moiety includes a “saturated alkyl” group, which means that it does not contain any alkene or alkyne moieties. The alkyl moiety also includes an “unsaturated alkyl” moiety, which means that it contains at least one alkene or alkyne moiety. An “alkene” moiety refers to a group that has at least one carbon-carbon double bond, and an “alkyne” moiety refers to a group that has at least one carbon-carbon triple bond. The alkyl moiety, whether saturated or unsaturated, includes branched, straight chain, or cyclic moieties. Depending on the structure, an alkyl group includes a monoradical or a diradical (i.e., an alkylene group), and if a “lower alkyl” having 1 to 6 carbon atoms.

As used herein, C₁-Cₓ includes C₁-C₂, C₁-C₃ . . . C₁-Cₓ.

The “alkyl” moiety optionally has 1 to 10 carbon atoms (whenever it appears herein, a numerical range such as “1 to 10” refers to each integer in the given range; e.g., “1 to 10 carbon atoms” means that the alkyl group is selected from a moiety having 1 carbon atom, 2 carbon atoms, 3 carbon atoms, etc., up to and including 10 carbon atoms, although the present definition also covers the occurrence of the term “alkyl” where no numerical range is designated). The alkyl group of the compounds described herein may be designated as “C₁-C₄ alkyl” or similar designations. By way of example only, “C₁-C₄ alkyl” indicates that there are one to four carbon atoms in the alkyl chain, i.e., the alkyl chain is selected from among methyl, ethyl, propyl, iso-propyl, n-butyl, iso-butyl, sec-butyl, and t-butyl. Thus C₁-C₄ alkyl includes C₁-C₂ alkyl and C₁-C₃ alkyl. Alkyl groups are optionally substituted or unsubstituted. Typical alkyl groups include, but are in no way limited to, methyl, ethyl, propyl, isopropyl, butyl, isobutyl, tertiary butyl, pentyl, hexyl, ethenyl, propenyl, butenyl, cyclopropyl, cyclobutyl, cyclopentyl, cyclohexyl, and the like.

The term “alkenyl” refers to a type of alkyl group in which the first two atoms of the alkyl group form a double bond that is not part of an aromatic group. That is, an alkenyl group begins with the atoms —C(R)═C(R)—R, wherein R refers to the remaining portions of the alkenyl group, which are either the same or different. The alkenyl moiety is optionally branched, straight chain, or cyclic (in which case, it is also known as a “cycloalkenyl” group). Depending on the structure, an alkenyl group includes a monoradical or a diradical (i.e., an alkenylene group). Alkenyl groups are optionally substituted. Non-limiting examples of an alkenyl group include —CH═CH₂, —C(CH₃)═CH₂, —CH═CHCH₃, —C(CH₃)═CHCH₃. Alkenylene groups include, but are not limited to, —CH═CH—, —C(CH₃)═CH—, —CH═CHCH₂—, —CH═CHCH₂CH₂— and —C(CH₃)═CHCH₂—. Alkenyl groups optionally have 2 to 10 carbons, and if a “lower alkenyl” having 2 to 6 carbon atoms.

The term “alkynyl” refers to a type of alkyl group in which the first two atoms of the alkyl group form a triple bond. That is, an alkynyl group begins with the atoms —C≡C—R, wherein R refers to the remaining portions of the alkynyl group, which is either the same or different. The “R” portion of the alkynyl moiety may be branched, straight chain, or cyclic. Depending on the structure, an alkynyl group includes a monoradical or a diradical (i.e., an alkynylene group). Alkynyl groups are optionally substituted. Non-limiting examples of an alkynyl group include, but are not limited to, —C≡CH, —C≡CCH₃, —C≡CCH₂CH₃, and —C≡CCH₂—. Alkynyl groups optionally have 2 to 10 carbons, and if a “lower alkynyl” having 2 to 6 carbon atoms.

An “alkoxy” group refers to a (alkyl)O— group, where alkyl is as defined herein.

“Hydroxyalkyl” refers to an alkyl radical, as defined herein, substituted with at least one hydroxy group. Non-limiting examples of a hydroxyalkyl include, but are not limited to, hydroxymethyl, 2-hydroxyethyl, 2-hydroxypropyl, 3-hydroxypropyl, 1-(hydroxymethyl)-2-methylpropyl, 2-hydroxybutyl, 3-hydroxybutyl, 4-hydroxybutyl, 2,3-dihydroxypropyl, 1-(hydroxymethyl)-2-hydroxyethyl, 2,3-dihydroxybutyl, 3,4-dihydroxybutyl and 2-(hydroxymethyl)-3-hydroxypropyl.

“Alkoxyalkyl” refers to an alkyl radical, as defined herein, substituted with an alkoxy group, as defined herein.

The term “alkylamine” refers to the —N(alkyl)ₓH_{y} group, where x and y are selected from among x=1, y=1 and x=2, y=0. When x=2, the alkyl groups, taken together with the N atom to which they are attached, optionally form a cyclic ring system.

“Alkylaminoalkyl” refers to an alkyl radical, as defined herein, substituted with an alkylamine, as defined herein.

“Hydroxyalkylaminoalkyl” refers to an alkyl radical, as defined herein, substituted with an alkylamine, and alkylhydroxy, as defined herein.

“Alkoxyalkylaminoalkyl” refers to an alkyl radical, as defined herein, substituted with an alkylamine and substituted with an alkylalkoxy, as defined herein.

An “amide” is a chemical moiety with the formula —C(O)NHR or —NHC(O)R, where R is selected from among alkyl, cycloalkyl, aryl, heteroaryl (bonded through a ring carbon) and heteroalicyclic (bonded through a ring carbon). In some embodiments, an amide moiety forms a linkage between an amino acid or a peptide molecule and a compound described herein, thereby forming a prodrug. Any amine, or carboxyl side chain on the compounds described herein can be amidified. The procedures and specific groups to make such amides are found in sources such as Greene and Wuts, Protective Groups in Organic Synthesis, 3^{rd} Ed., John Wiley & Sons, New York, N.Y., 1999, which is incorporated herein by reference for this disclosure.

The term “ester” refers to a chemical moiety with formula —COOR, where R is selected from among alkyl, cycloalkyl, aryl, heteroaryl (bonded through a ring carbon) and heteroalicyclic (bonded through a ring carbon). Any hydroxy, or carboxyl side chain on the compounds described herein can be esterified. The procedures and specific groups to make such esters are found in sources such as Greene and Wuts, Protective Groups in Organic Synthesis, 3^{rd} Ed., John Wiley & Sons, New York, N.Y., 1999, which is incorporated herein by reference for this disclosure.

As used herein, the term “ring” refers to any covalently closed structure. Rings include, for example, carbocycles (e.g., aryls and cycloalkyls), heterocycles (e.g., heteroaryls and non-aromatic heterocycles), aromatics (e.g. aryls and heteroaryls), and non-aromatics (e.g., cycloalkyls and non-aromatic heterocycles). Rings can be optionally substituted. Rings can be monocyclic or polycyclic.

As used herein, the term “ring system” refers to one, or more than one ring.

The term “membered ring” can embrace any cyclic structure. The term “membered” is meant to denote the number of skeletal atoms that constitute the ring. Thus, for example, cyclohexyl, pyridine, pyran and thiopyran are 6-membered rings and cyclopentyl, pyrrole, furan, and thiophene are 5-membered rings.

The term “fused” refers to structures in which two or more rings share one or more bonds.

The term “carbocyclic” or “carbocycle” refers to a ring wherein each of the atoms forming the ring is a carbon atom. Carbocycle includes aryl and cycloalkyl. The term thus distinguishes carbocycle from heterocycle (“heterocyclic”) in which the ring backbone contains at least one atom which is different from carbon (i.e. a heteroatom). Heterocycle includes heteroaryl and heterocycloalkyl. Carbocycles and heterocycles can be optionally substituted.

The term “aromatic” refers to a planar ring having a delocalized π-electron system containing 4n+2π electrons, where n is an integer. Aromatic rings can be formed from five, six, seven, eight, nine, or more than nine atoms. Aromatics can be optionally substituted. The term “aromatic” includes both carbocyclic aryl (e.g., phenyl) and heterocyclic aryl (or “heteroaryl” or “heteroaromatic”) groups (e.g., pyridine). The term includes monocyclic or fused-ring polycyclic (i.e., rings which share adjacent pairs of carbon atoms) groups.

As used herein, the term “aryl” refers to an aromatic ring wherein each of the atoms forming the ring is a carbon atom. Aryl rings can be formed by five, six, seven, eight, nine, or more than nine carbon atoms. Aryl groups can be optionally substituted. Examples of aryl groups include, but are not limited to phenyl, naphthalenyl, phenanthrenyl, anthracenyl, fluorenyl, and indenyl. Depending on the structure, an aryl group can be a monoradical or a diradical (i.e., an arylene group).

An “aryloxy” group refers to an (aryl)O— group, where aryl is as defined herein.

The term “carbonyl” as used herein refers to a group containing a moiety selected from the group consisting of —C(O)—, —S(O)—, —S(O)2-, and —C(S)—, including, but not limited to, groups containing a least one ketone group, and/or at least one aldehyde group, and/or at least one ester group, and/or at least one carboxylic acid group, and/or at least one thioester group. Such carbonyl groups include ketones, aldehydes, carboxylic acids, esters, and thioesters. In some embodiments, such groups are a part of linear, branched, or cyclic molecules.

The term “cycloalkyl” refers to a monocyclic or polycyclic radical that contains only carbon and hydrogen, and is optionally saturated, partially unsaturated, or fully unsaturated. Cycloalkyl groups include groups having from 3 to 10 ring atoms. Illustrative examples of cycloalkyl groups include the following moieties:

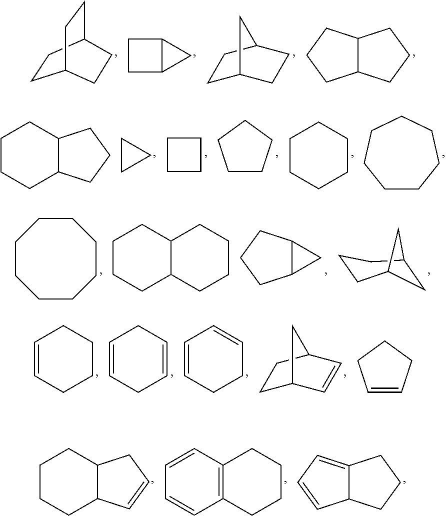

and the like. Depending on the structure, a cycloalkyl group is either a monoradical or a diradical (e.g., an cycloalkylene group), and if a “lower cycloalkyl” having 3 to 8 carbon atoms.

“Cycloalkylalkyl” means an alkyl radical, as defined herein, substituted with a cycloalkyl group. Non-limiting cycloalkylalkyl groups include cyclopropylmethyl, cyclobutylmethyl, cyclopentylmethyl, cyclohexylmethyl, and the like.

The term “heterocycle” refers to heteroaromatic and heteroalicyclic groups containing one to four heteroatoms each selected from O, S and N, wherein each heterocyclic group has from 4 to 10 atoms in its ring system, and with the proviso that the ring of said group does not contain two adjacent O or S atoms. Herein, whenever the number of carbon atoms in a heterocycle is indicated (e.g., C₁-C₆ heterocycle), at least one other atom (the heteroatom) must be present in the ring. Designations such as “C₁-C₆ heterocycle” refer only to the number of carbon atoms in the ring and do not refer to the total number of atoms in the ring. It is understood that the heterocylic ring can have additional heteroatoms in the ring. Designations such as “4-6 membered heterocycle” refer to the total number of atoms that are contained in the ring (i.e., a four, five, or six membered ring, in which at least one atom is a carbon atom, at least one atom is a heteroatom and the remaining two to four atoms are either carbon atoms or heteroatoms). In heterocycles that have two or more heteroatoms, those two or more heteroatoms can be the same or different from one another. Heterocycles can be optionally substituted. Binding to a heterocycle can be at a heteroatom or via a carbon atom. Non-aromatic heterocyclic groups include groups having only 4 atoms in their ring system, but aromatic heterocyclic groups must have at least 5 atoms in their ring system. The heterocyclic groups include benzo-fused ring systems. An example of a 4-membered heterocyclic group is azetidinyl (derived from azetidine). An example of a 5-membered heterocyclic group is thiazolyl. An example of a 6-membered heterocyclic group is pyridyl, and an example of a 10-membered heterocyclic group is quinolinyl. Examples of non-aromatic heterocyclic groups are pyrrolidinyl, tetrahydrofuranyl, dihydrofuranyl, tetrahydrothienyl, tetrahydropyranyl, dihydropyranyl, tetrahydrothiopyranyl, piperidino, morpholino, thiomorpholino, thioxanyl, piperazinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl and quinolizinyl. Examples of aromatic heterocyclic groups are pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. The foregoing groups, as derived from the groups listed above, are optionally C-attached or N-attached where such is possible. For instance, a group derived from pyrrole includes pyrrol-1-yl (N-attached) or pyrrol-3-yl (C-attached). Further, a group derived from imidazole includes imidazol-1-yl or imidazol-3-yl (both N-attached) or imidazol-2-yl, imidazol-4-yl or imidazol-5-yl (all C-attached). The heterocyclic groups include benzo-fused ring systems and ring systems substituted with one or two oxo (═O) moieties such as pyrrolidin-2-one. Depending on the structure, a heterocycle group can be a monoradical or a diradical (i.e., a heterocyclene group).

The terms “heteroaryl” or, alternatively, “heteroaromatic” refers to an aromatic group that includes one or more ring heteroatoms selected from nitrogen, oxygen and sulfur. An N-containing “heteroaromatic” or “heteroaryl” moiety refers to an aromatic group in which at least one of the skeletal atoms of the ring is a nitrogen atom. Illustrative examples of heteroaryl groups include the following moieties:

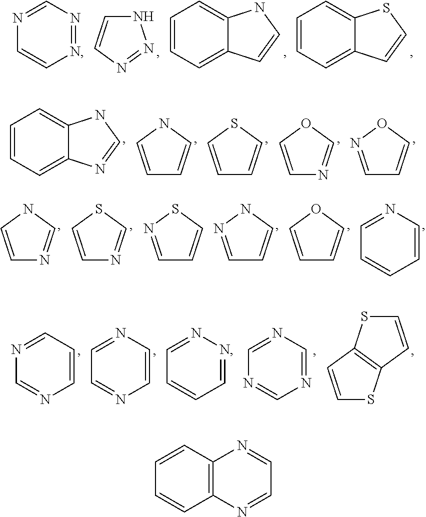

and the like. Depending on the structure, a heteroaryl group can be a monoradical or a diradical (i.e., a heteroarylene group).

As used herein, the term “non-aromatic heterocycle”, “heterocycloalkyl” or “heteroalicyclic” refers to a non-aromatic ring wherein one or more atoms forming the ring is a heteroatom. A “non-aromatic heterocycle” or “heterocycloalkyl” group refers to a cycloalkyl group that includes at least one heteroatom selected from nitrogen, oxygen and sulfur. In some embodiments, the radicals are fused with an aryl or heteroaryl. Heterocycloalkyl rings can be formed by three, four, five, six, seven, eight, nine, or more than nine atoms. Heterocycloalkyl rings can be optionally substituted. In certain embodiments, non-aromatic heterocycles contain one or more carbonyl or thiocarbonyl groups such as, for example, oxo- and thio-containing groups. Examples of heterocycloalkyls include, but are not limited to, lactams, lactones, cyclic imides, cyclic thioimides, cyclic carbamates, tetrahydrothiopyran, 4H-pyran, tetrahydropyran, piperidine, 1,3-dioxin, 1,3-dioxane, 1,4-dioxin, 1,4-dioxane, piperazine, 1,3-oxathiane, 1,4-oxathiin, 1,4-oxathiane, tetrahydro-1,4-thiazine, 2H-1,2-oxazine, maleimide, succinimide, barbituric acid, thiobarbituric acid, dioxopiperazine, hydantoin, dihydrouracil, morpholine, trioxane, hexahydro-1,3,5-triazine, tetrahydrothiophene, tetrahydrofuran, pyrroline, pyrrolidine, pyrrolidone, pyrrolidione, pyrazoline, pyrazolidine, imidazoline, imidazolidine, 1,3-dioxole, 1,3-dioxolane, 1,3-dithiole, 1,3-dithiolane, isoxazoline, isoxazolidine, oxazoline, oxazolidine, oxazolidinone, thiazoline, thiazolidine, and 1,3-oxathiolane. Illustrative examples of heterocycloalkyl groups, also referred to as non-aromatic heterocycles, include:

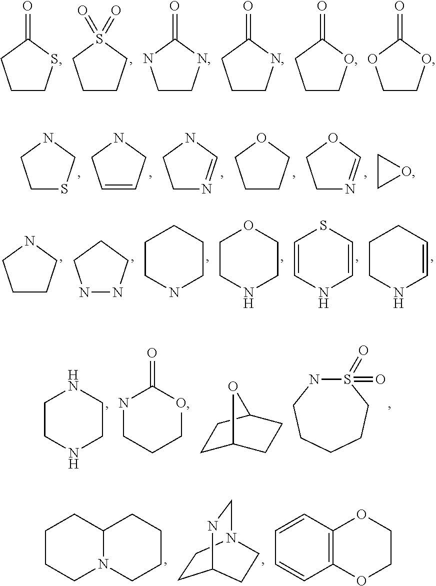

and the like. The term heteroalicyclic also includes all ring forms of the carbohydrates, including but not limited to the monosaccharides, the disaccharides and the oligosaccharides. Depending on the structure, a heterocycloalkyl group can be a monoradical or a diradical (i.e., a heterocycloalkylene group).

The term “halo” or, alternatively, “halogen” or “halide” means fluoro, chloro, bromo and iodo.

The term “haloalkyl,” refers to alkyl structures in which at least one hydrogen is replaced with a halogen atom. In certain embodiments in which two or more hydrogen atoms are replaced with halogen atoms, the halogen atoms are all the same as one another. In other embodiments in which two or more hydrogen atoms are replaced with halogen atoms, the halogen atoms are not all the same as one another.

The term “fluoroalkyl,” as used herein, refers to alkyl group in which at least one hydrogen is replaced with a fluorine atom. Examples of fluoroalkyl groups include, but are not limited to, —CF₃, —CH₂CF₃, —CF₂CF₃, —CH₂CH₂CF₃ and the like.

As used herein, the term “heteroalkyl” refers to optionally substituted alkyl radicals in which one or more skeletal chain atoms is a heteroatom, e.g., oxygen, nitrogen, sulfur, silicon, phosphorus or combinations thereof. The heteroatom(s) are placed at any interior position of the heteroalkyl group or at the position at which the heteroalkyl group is attached to the remainder of the molecule. Examples include, but are not limited to, —CH₂—O—CH₃, —CH₂—CH₂—O—CH₃, —CH₂—NH—CH₃, —CH₂—CH₂—NH—CH₃, —CH₂—N(CH₃)—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, and —CH═CH—N(CH₃)—CH₃. In addition, in some embodiments, up to two heteroatoms are consecutive, such as, by way of example, —CH₂—NH—OCH₃ and —CH₂—O—Si(CH₃)₃.

The term “heteroatom” refers to an atom other than carbon or hydrogen. Heteroatoms are typically independently selected from among oxygen, sulfur, nitrogen, silicon and phosphorus, but are not limited to these atoms. In embodiments in which two or more heteroatoms are present, the two or more heteroatoms can all be the same as one another, or some or all of the two or more heteroatoms can each be different from the others.

The term “bond” or “single bond” refers to a chemical bond between two atoms, or two moieties when the atoms joined by the bond are considered to be part of larger substructure.

The term “moiety” refers to a specific segment or functional group of a molecule. Chemical moieties are often recognized chemical entities embedded in or appended to a molecule.

A “thioalkoxy” or “alkylthio” group refers to a —S-alkyl group.

A “SH” group is also referred to either as a thiol group or a sulfhydryl group.

The term “optionally substituted” or “substituted” means that the referenced group may be substituted with one or more additional group(s) individually and independently selected from alkyl, cycloalkyl, aryl, heteroaryl, heteroalicyclic, hydroxy, alkoxy, aryloxy, alkylthio, arylthio, alkylsulfoxide, arylsulfoxide, alkylsulfone, arylsulfone, cyano, halo, acyl, nitro, haloalkyl, fluoroalkyl, amino, including mono- and di-substituted amino groups, and the protected derivatives thereof. By way of example an optional substituents may be LₛRₛ, wherein each L, is independently selected from a bond, —O—, —C(═O)—, —S—, —S(═O)—, —S(═O)₂—, —NH—, —NHC(O)—, —C(O)NH—, S(═O)₂NH—, —NHS(═O)₂, —OC(O)NH—, —NHC(O)O—, -(substituted or unsubstituted C₁-C₆ alkyl), or -(substituted or unsubstituted C₂-C₆ alkenyl); and each Rₛ is independently selected from H, (substituted or unsubstituted C₁-C₄alkyl), (substituted or unsubstituted C₃-C₆cycloalkyl), heteroaryl, or heteroalkyl. The protecting groups that form the protective derivatives of the above substituents include those found in sources such as Greene and Wuts, above.

### Covalent BTK Inhibitor Compounds

Described herein methods of preventing or delaying the onset of Type 1 Diabetes, inhibiting the maturation of anti-insulin B cells, or decreasing the population of anti-insulin B cells, in an individual in need thereof comprising administering to the individual a composition comprising a therapeutically-effective amount of a covalent Btk inhibitor (e.g., an irreversible covalent Btk inhibitor, e.g., ibrutinib).

In some embodiments, the covalent Btk inhibitor is a compound of Formula (A):

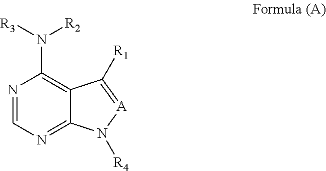

wherein
   A is independently selected from N or CR₅;
   R₁ is H, L₂-(substituted or unsubstituted alkyl), L₂-(substituted or unsubstituted cycloalkyl), L₂-(substituted or unsubstituted alkenyl), L₂-(substituted or unsubstituted cycloalkenyl), L₂-(substituted or unsubstituted heterocycle), L₂-(substituted or unsubstituted heteroaryl), or L₂-(substituted or unsubstituted aryl), where L₂ is a bond, O, S, —S(═O), —S(═O)₂, C(═O), -(substituted or unsubstituted C₁-C₆ alkyl), or -(substituted or unsubstituted C₂-C₆ alkenyl);
   R₂ and R₃ are independently selected from H, lower alkyl and substituted lower alkyl;
   R₄ is L₃-X-L₄-G, wherein,
      L₃ is optional, and when present is a bond, optionally substituted or unsubstituted alkyl, optionally substituted or unsubstituted cycloalkyl, optionally substituted or unsubstituted alkenyl, optionally substituted or unsubstituted alkynyl;
      X is optional, and when present is a bond, O, —C(═O), S, —S(═O), —S(═O)₂, —NH, —NR₉, —NHC(O), —C(O)NH, —NR₉C(O), —C(O)NR₉, —S(═O)₂NH, —NHS(═O)₂, —S(═O)₂NR₉—, —NR₉S(═O)₂, —OC(O)NH—, —NHC(O)O—, —OC(O)NR₉—, —NR₉C(O)O—, —CH═NO—, —ON═CH—, —NR₁₀C(O)NR₁₀—, heteroaryl, aryl, —NR₁₀C(═NR₁₁)NR₁₀—, —NR₁₀C(═NR₁₁)—, —C(═NR₁₁)NR₁₀—, —OC(═NR₁₁)—, or —C(═NR₁₁)O—;
      L₄ is optional, and when present is a bond, substituted or unsubstituted alkyl, substituted or unsubstituted cycloalkyl, substituted or unsubstituted alkenyl, substituted or unsubstituted alkynyl, substituted or unsubstituted aryl, substituted or unsubstituted heteroaryl, substituted or unsubstituted heterocycle;
      or L₃, X and L₄ taken together form a nitrogen containing heterocyclic ring;
      G is

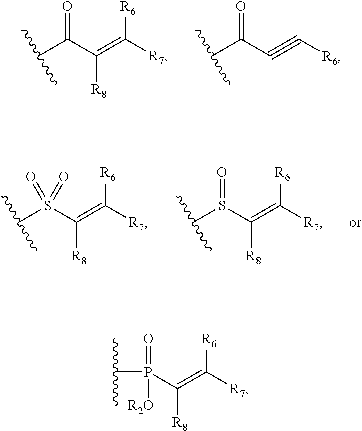

wherein,
   R₆, R₇ and R₈ are independently selected from among H, lower alkyl or substituted lower alkyl, lower heteroalkyl or substituted lower heteroalkyl, substituted or unsubstituted lower cycloalkyl, and substituted or unsubstituted lower heterocycloalkyl;
R₅ is H, halogen, -L₆-(substituted or unsubstituted C₁-C₃ alkyl), -L₆-(substituted or unsubstituted C₂-C₄ alkenyl), -L₆-(substituted or unsubstituted heteroaryl), or -L₆-(substituted or unsubstituted aryl), wherein L₆ is a bond, O, S, —S(═O), S(═O)₂, NH, C(O), —NHC(O)O, —OC(O)NH, —NHC(O), or —C(O)NH;
each R₉ is independently selected from among H, substituted or unsubstituted lower alkyl, and substituted or unsubstituted lower cycloalkyl;
each R₁₀ is independently H, substituted or unsubstituted lower alkyl, or substituted or unsubstituted lower cycloalkyl; or
two R₁₀ groups can together form a 5-, 6-, 7-, or 8-membered heterocyclic ring; or
R₁₀ and R₁₁ can together form a 5-, 6-, 7-, or 8-membered heterocyclic ring; or
each R₁₁ is independently selected from H or alkyl; and
pharmaceutically active metabolites, pharmaceutically acceptable solvates, pharmaceutically acceptable salts, or pharmaceutically acceptable prodrugs thereof.

In one embodiment, the compound of Formula (A) has the structure:

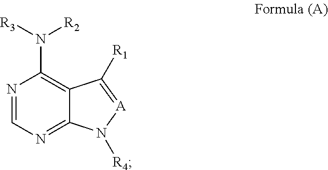

wherein:
A is N;
R₂ and R₃ are each H;
R₁ is phenyl-O-phenyl or phenyl-S-phenyl; and
R₄ is L₃-X-L₄-G, wherein,
   L₃ is optional, and when present is a bond, optionally substituted or unsubstituted alkyl, optionally substituted or unsubstituted cycloalkyl, optionally substituted or unsubstituted alkenyl, optionally substituted or unsubstituted alkynyl;
   X is optional, and when present is a bond, O, —C(═O), S, —S(═O), —S(═O)₂, —NH, —NR₉, —NHC(O), —C(O)NH, —NR₉C(O), —C(O)NR₉, —S(═O)₂NH, —NHS(═O)₂, —S(═O)₂NR₉—, —NR₉S(═O)₂, —OC(O)NH—, —NHC(O)O—, —OC(O)NR₉—, —NR₉C(O)O—, —CH═NO—, —ON═CH—, —NR₁₀C(O)NR₁₀—, heteroaryl, aryl, —NR₁₀C(═NR₁₁)NR₁₀—, —NR₁₀C(═NR₁₁)—, —C(═NR₁₁)NR₁₀—, —OC(═NR₁₁)—, or —C(═NR₁₁)O—;
   L₄ is optional, and when present is a bond, substituted or unsubstituted alkyl, substituted or unsubstituted cycloalkyl, substituted or unsubstituted alkenyl, substituted or unsubstituted alkynyl, substituted or unsubstituted aryl, substituted or unsubstituted heteroaryl, substituted or unsubstituted heterocycle;
   or L₃, X and L₄ taken together form a nitrogen containing heterocyclic ring;
   G is

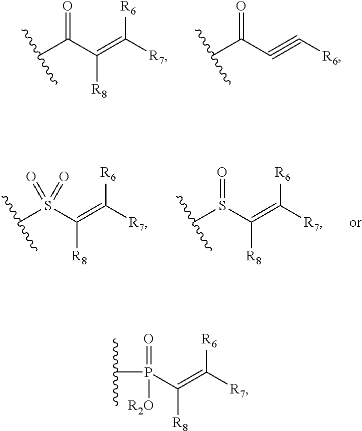

wherein,
R₆, R₇ and R₈ are independently selected from among H, lower alkyl or substituted lower alkyl, lower heteroalkyl or substituted lower heteroalkyl, substituted or unsubstituted lower cycloalkyl, and substituted or unsubstituted lower heterocycloalkyl.

In some embodiments, the covalent BTK inhibitor is (R)-1-(3-(4-amino-3-(4-phenoxyphenyl)-1H-pyrazolo[3,4-d]pyrimidin-1-yl)piperidin-1-yl)prop-2-en-1-one (i.e. PCI-32765/ibrutinib)

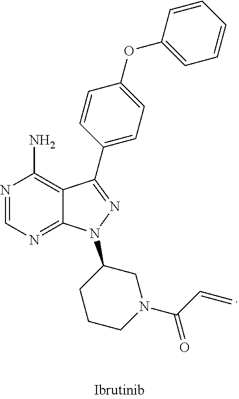 [SMILES: C=CC(=O)N1CCC[C@@H](n2nc(-c3ccc(Oc4ccccc4)cc3)c3c(N)ncnc32)C1]

Further examples of Btk inhibitors may be found in the following patents and patent applications, all of which are incorporated herein in their entirety by reference: U.S. Pat. No. 7,514,444; U.S. Pat. No. 7,960,396; U.S. Pat. No. 8,236,812; U.S. Pat. No. 8,497,277; U.S. Pat. No. 8,563,563; U.S. Pat. No. 8,399,470; U.S. Pat. No. 8,088,781; U.S. Pat. No. 8,501,751; U.S. Pat. No. 8,008,309; U.S. Pat. No. 8,552,010; U.S. Pat. No. 7,732,454; U.S. Pat. No. 7,825,118; U.S. Pat. No. 8,377,946; U.S. Pat. No. 8,501,724; US Patent Pub. No. 2011-0039868; U.S. Pat. No. 8,232,280; U.S. Pat. No. 8,158,786; US Patent Pub. No. 2011-0281322; US Patent Pub. No. 2012-0088912; US Patent Pub. No. 2012-0108612; US Patent Pub. No. 2012-0115889; US Patent Pub. No. 2013-0005745; US Patent Pub. No. 2012-0122894; US Patent Pub. No. 2012-0135944; US Patent Pub. No. 2012-0214826; US Patent Pub. No. 2012-0252821; US Patent Pub. No. 2012-0252822; US Patent Pub. No. 2012-0277254; US Patent Pub. No. 2010-0022561; US Patent Pub. No. 2010-0324050; US Patent Pub. No. 2012-0283276; US Patent Pub. No. 2012-0065201; US Patent Pub. No. 2012-0178753; US Patent Pub. No. 2012-0101113; US Patent Pub. No. 2012-0101114; US Patent Pub. No. 2012-0165328; US Patent Pub. No. 2012-0184013; US Patent Pub. No. 2012-0184567; US Patent Pub. No. 2012-0202264; US Patent Pub. No. 2012-0277225; US Patent Pub. No. 2012-0277255; US Patent Pub. No. 2012-0296089; US Patent Pub. No. 2013-0035334; US Patent Pub. No. 2012-0329130; US Patent Pub. No. 2013-0018060; US Patent Pub. No. 2010-0254905; U.S. Patent App. No. 60/826,720; U.S. Patent App. No. 60/828,590; U.S. patent application Ser. No. 13/654,173; U.S. patent application Ser. No. 13/849,399; U.S. patent application Ser. No. 13/890,498; U.S. patent application Ser. No. 13/952,531; U.S. patent application Ser. No. 14/033,344; U.S. patent application Ser. No. 14/073,543; U.S. patent application Ser. No. 14/073,594; U.S. patent application Ser. No. 14/079,508; U.S. patent application Ser. No. 14/080,640; U.S. patent application Ser. No. 14/080,649; U.S. patent application Ser. No. 14/069,222; PCT App. No. PCT/US2008/58528; PCT App. No. PCT/US2012/046779; U.S. Patent App. No. 61/582,199; U.S. patent application Ser. No. 13/619,466; PCT App. No. PCT/US2012/72043; U.S. Patent App. No. 61/593,146; U.S. Patent App. No. 61/637,765; PCT App. No. PCT/US2013/23918; U.S. Patent App. No. 61/781,975; U.S. Patent App. No. 61/727,031; PCT App. No. PCT/US2013/7016; U.S. Patent App. No. 61/647,956; PCT App. No. PCT/US2013/41242; U.S. Patent App. No. 61/769,103; U.S. Patent App. No. 61/842,321; and U.S. Patent App. No. 61/884,888.

Additional examples of Btk inhibitors include PCI-45292, PCI-45466, AVL-101/CC-101 (Avila Therapeutics/Celgene Corporation), AVL-263/CC-263 (Avila Therapeutics/Celgene Corporation), AVL-292/CC-292 (Avila Therapeutics/Celgene Corporation), AVL-291/CC-291 (Avila Therapeutics/Celgene Corporation), CNX 774 (Avila Therapeutics), BMS-488516 (Bristol-Myers Squibb), BMS-509744 (Bristol-Myers Squibb), CGI-1746 (CGI Pharma/Gilead Sciences), CGI-560 (CGI Pharma/Gilead Sciences), CTA-056, GDC-0834 (Genentech), HY-11066 (also, CTK4I7891, HMS3265G21, HMS3265G22, HMS3265H21, HMS3265H22, 439574-61-5, AG-F-54930), ONO-4059 (Ono Pharmaceutical Co., Ltd.), ONO-WG37 (Ono Pharmaceutical Co., Ltd.), PLS-123 (Peking University), RN486 (Hoffmann-La Roche), HM71224 (Hanmi Pharmaceutical Company Limited), LFM-A13, BGB-3111 (Beigene), KBP-7536 (KBP BioSciences), ACP-196 (Acerta Pharma) and JTE-051 (Japan Tobacco Inc).

### Additional TEC Family Kinase Inhibitors

BTK is a member of the Tyrosine-protein kinase (TEC) family of kinases. In some embodiments, the TEC family comprises BTK, ITK, TEC, RLK and BMX. In some embodiments, a covalent TEC family kinase inhibitor inhibits the kinase activity of BTK, ITK, TEC, RLK and BMX. In some embodiments, a covalent TEC family kinase inhibitor is a BTK inhibitor, which is disclosed elsewhere herein. In some embodiments, a covalent TEC family kinase inhibitor is an ITK inhibitor. In some embodiments, a covalent TEC family kinase inhibitor is a TEC inhibitor. In some embodiments, a covalent TEC family kinase inhibitor is a RLK inhibitor. In some embodiments, a covalent TEC family kinase inhibitor is a BMK inhibitor.

In some embodiments, the ITK inhibitor covalently binds to Cysteine 442 of ITK. In some embodiments, the Itk inhibitor is an Itk inhibitor compound described in WO2002/0500071, which is incorporated by reference in its entirety. In some embodiments, the Itk inhibitor is an Itk inhibitor compound described in WO2005/070420, which is incorporated by reference in its entirety. In some embodiments, the Itk inhibitor is an Itk inhibitor compound described in WO2005/079791, which is incorporated by reference in its entirety. In some embodiments, the Itk inhibitor is an Itk inhibitor compound described in WO2007/076228, which is incorporated by reference in its entirety. In some embodiments, the Itk inhibitor is an Itk inhibitor compound described in WO2007/058832, which is incorporated by reference in its entirety. In some embodiments, the Itk inhibitor is an Itk inhibitor compound described in WO2004/016610, which is incorporated by reference in its entirety. In some embodiments, the Itk inhibitor is an Itk inhibitor compound described in WO2004/016611, which is incorporated by reference in its entirety. In some embodiments, the Itk inhibitor is an Itk inhibitor compound described in WO2004/016600, which is incorporated by reference in its entirety. In some embodiments, the Itk inhibitor is an Itk inhibitor compound described in WO2004/016615, which is incorporated by reference in its entirety. In some embodiments, the Itk inhibitor is an Itk inhibitor compound described in WO2005/026175, which is incorporated by reference in its entirety. In some embodiments, the Itk inhibitor is an Itk inhibitor compound described in WO2006/065946, which is incorporated by reference in its entirety. In some embodiments, the Itk inhibitor is an Itk inhibitor compound described in WO2007/027594, which is incorporated by reference in its entirety. In some embodiments, the Itk inhibitor is an Itk inhibitor compound described in WO2007/017455, which is incorporated by reference in its entirety. In some embodiments, the Itk inhibitor is an Itk inhibitor compound described in WO2008/025820, which is incorporated by reference in its entirety. In some embodiments, the Itk inhibitor is an Itk inhibitor compound described in WO2008/025821, which is incorporated by reference in its entirety. In some embodiments, the Itk inhibitor is an Itk inhibitor compound described in WO2008/025822, which is incorporated by reference in its entirety. In some embodiments, the Itk inhibitor is an Itk inhibitor compound described in WO2011/017219, which is incorporated by reference in its entirety. In some embodiments, the Itk inhibitor is an Itk inhibitor compound described in WO2011/090760, which is incorporated by reference in its entirety. In some embodiments, the Itk inhibitor is an Itk inhibitor compound described in WO2009/158571, which is incorporated by reference in its entirety. In some embodiments, the Itk inhibitor is an Itk inhibitor compound described in WO2009/051822, which is incorporated by reference in its entirety. In some embodiments, the Itk inhibitor is an Itk inhibitor compound described in US20110281850, which is incorporated by reference in its entirety. In some embodiments, the Itk inhibitor is an Itk inhibitor compound described in WO2014/082085, which is incorporated by reference in its entirety. In some embodiments, the Itk inhibitor is an Itk inhibitor compound described in WO2014/093383, which is incorporated by reference in its entirety. In some embodiments, the Itk inhibitor is an Itk inhibitor compound described in U.S. Pat. No. 8,759,358, which is incorporated by reference in its entirety. In some embodiments, the Itk inhibitor is an Itk inhibitor compound described in WO2014/105958, which is incorporated by reference in its entirety. In some embodiments, the Itk inhibitor is an Itk inhibitor compound described in US2014/0256704, which is incorporated by reference in its entirety. In some embodiments, the Itk inhibitor is an Itk inhibitor compound described in US20140315909, which is incorporated by reference in its entirety. In some embodiments, the Itk inhibitor is an Itk inhibitor compound described in US20140303161, which is incorporated by reference in its entirety. In some embodiments, the Itk inhibitor is an Itk inhibitor compound described in WO2014/145403, which is incorporated by reference in its entirety.

In some embodiments, the Itk inhibitor has a structure selected from:

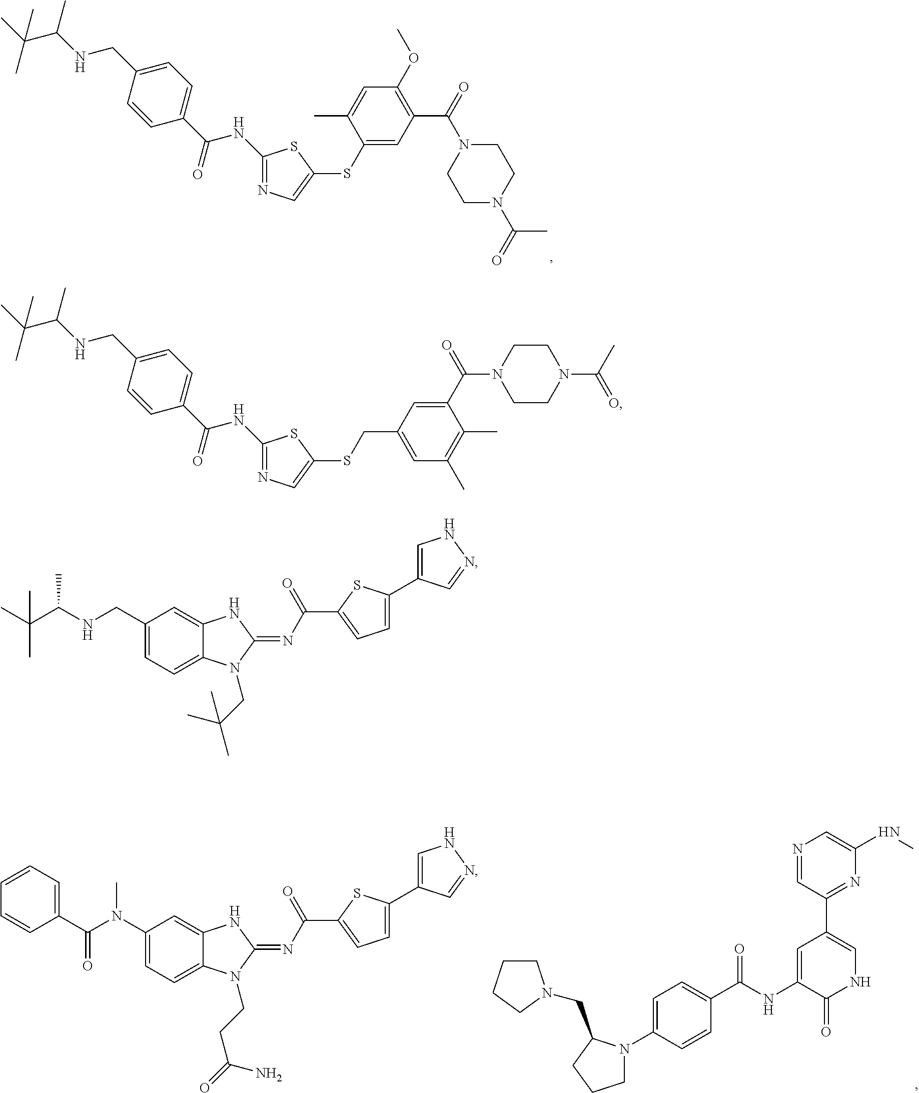

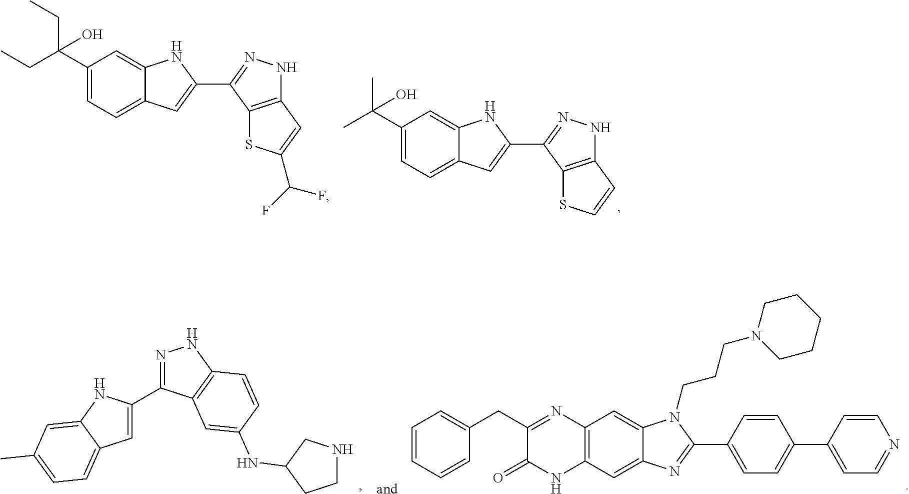

### Pharmaceutical Compositions/Formulations

Disclosed herein, in certain embodiments, are compositions comprising a therapeutically effective amount of a Tec inhibitor, and a pharmaceutically acceptable excipient for use in preventing or delaying the onset of Type 1 Diabetes, inhibiting the maturation of anti-insulin B cells, or decreasing the population of anti-insulin B cells, in an individual in need thereof. In some instances, the Tec inhibitor is a Btk inhibitor or an ITK inhibitor. In some instances, the Tec inhibitor is a Btk inhibitor. In some instances, the Btk inhibitor is a covalent Btk inhibitor. In some embodiments, described herein are compositions comprising a therapeutically effective amount of a covalent Btk inhibitor (e.g., an irreversible covalent Btk inhibitor, e.g., ibrutinib), and a pharmaceutically acceptable excipient for use in preventing or delaying the onset of Type 1 Diabetes, inhibiting the maturation of anti-insulin B cells, or decreasing the population of anti-insulin B cells, in an individual in need thereof. In some embodiments, the covalent Btk inhibitor is a compound of Formula (A). In some embodiments, the covalent Btk inhibitor is (R)-1-(3-(4-amino-3-(4-phenoxyphenyl)-1H-pyrazolo[3,4-dlpyrimidin-1-yl]piperidin-1-yl)prop-2-en-1-one (i.e. PCI-32765/ibrutinib).

Pharmaceutical compositions of covalent Btk inhibitors (e.g., an irreversible covalent Btk inhibitor, e.g., ibrutinib) are formulated in a conventional manner using one or more physiologically acceptable carriers including excipients and auxiliaries which facilitate processing of the active compounds into preparations which can be used pharmaceutically. Proper formulation is dependent upon the route of administration chosen. A summary of pharmaceutical compositions described herein is found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999).

A pharmaceutical composition, as used herein, refers to a mixture of a covalent Btk inhibitor (e.g., an irreversible covalent Btk inhibitor, e.g., ibrutinib) with other chemical components, such as carriers, stabilizers, diluents, dispersing agents, suspending agents, thickening agents, and/or excipients.

Pharmaceutical compositions are optionally manufactured in a conventional manner, such as, by way of example only, by means of conventional mixing, dissolving, granulating, dragee-making, levigating, emulsifying, encapsulating, entrapping or compression processes.

In some embodiments, the pharmaceutical combination and/or composition described herein also include one or more pH adjusting agents or buffering agents, including acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

In some embodiments, the pharmaceutical compositions also include one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

The pharmaceutical formulations described herein can be administered to a subject by multiple administration routes, including but not limited to, oral, parenteral (e.g., intravenous, subcutaneous, intramuscular), intranasal, buccal, topical, rectal, or transdermal administration routes. The pharmaceutical formulations described herein include, but are not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations, and mixed immediate and controlled release formulations.

In some embodiments, pharmaceutical compositions including a compound described herein are manufactured in a conventional manner, such as, by way of example only, by means of conventional mixing, dissolving, granulating, dragee-making, levigating, emulsifying, encapsulating, entrapping or compression processes.

“Antifoaming agents” reduce foaming during processing which can result in coagulation of aqueous dispersions, bubbles in the finished film, or generally impair processing. Exemplary anti-foaming agents include silicon emulsions or sorbitan sesquoleate.

“Antioxidants” include, for example, butylated hydroxytoluene (BHT), sodium ascorbate, ascorbic acid, sodium metabisulfite and tocopherol. In certain embodiments, antioxidants enhance chemical stability where required.

In some embodiments, compositions provided herein also include one or more preservatives to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide and cetylpyridinium chloride.

In some embodiments, formulations described herein benefit from antioxidants, metal chelating agents, thiol containing compounds and other general stabilizing agents. Examples of such stabilizing agents, include, but are not limited to: (a) about 0.5% to about 2% w/v glycerol, (b) about 0.1% to about 1% w/v methionine, (c) about 0.1% to about 2% w/v monothioglycerol, (d) about 1 mM to about 10 mM EDTA, (e) about 0.01% to about 2% w/v ascorbic acid, (f) 0.003% to about 0.02% w/v polysorbate 80, (g) 0.001% to about 0.05% w/v. polysorbate 20, (h) arginine, (i) heparin, (j) dextran sulfate, (k) cyclodextrins, (l) pentosan polysulfate and other heparinoids, (m) divalent cations such as magnesium and zinc; or (n) combinations thereof.

“Binders” impart cohesive qualities and include, e.g., alginic acid and salts thereof; cellulose derivatives such as carboxymethylcellulose, methylcellulose (e.g., Methocel®), hydroxypropylmethylcellulose, hydroxyethylcellulose, hydroxypropylcellulose (e.g., Klucer), ethylcellulose (e.g., Ethocel®), and microcrystalline cellulose (e.g., Avicel®); microcrystalline dextrose; amylose; magnesium aluminum silicate; polysaccharide acids; bentonites; gelatin; polyvinylpyrrolidone/vinyl acetate copolymer; crospovidone; povidone; starch; pregelatinized starch; tragacanth, dextrin, a sugar, such as sucrose (e.g., Dipac®), glucose, dextrose, molasses, mannitol, sorbitol, xylitol (e.g., Xylitab®), and lactose; a natural or synthetic gum such as acacia, tragacanth, ghatti gum, mucilage of isapol husks, polyvinylpyrrolidone (e.g., Polyvidone® CL, Kollidon® CL, Polyplasdone® XL-10), larch arabogalactan, Veegum®, polyethylene glycol, waxes, sodium alginate, and the like.

A “carrier” or “carrier materials” include any commonly used excipients in pharmaceutics and should be selected on the basis of compatibility with compounds disclosed herein, such as, compounds of ibrutinib, and the release profile properties of the desired dosage form. Exemplary carrier materials include, e.g., binders, suspending agents, disintegration agents, filling agents, surfactants, solubilizers, stabilizers, lubricants, wetting agents, diluents, and the like. “Pharmaceutically compatible carrier materials” include, but are not limited to, acacia, gelatin, colloidal silicon dioxide, calcium glycerophosphate, calcium lactate, maltodextrin, glycerine, magnesium silicate, polyvinylpyrrollidone (PVP), cholesterol, cholesterol esters, sodium caseinate, soy lecithin, taurocholic acid, phosphotidylcholine, sodium chloride, tricalcium phosphate, dipotassium phosphate, cellulose and cellulose conjugates, sugars sodium stearoyl lactylate, carrageenan, monoglyceride, diglyceride, pregelatinized starch, and the like. See, e.g., Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999).

“Dispersing agents,” and/or “viscosity modulating agents” include materials that control the diffusion and homogeneity of a drug through liquid media or a granulation method or blend method. In some embodiments, these agents also facilitate the effectiveness of a coating or eroding matrix. Exemplary diffusion facilitators/dispersing agents include, e.g., hydrophilic polymers, electrolytes, Tween® 60 or 80, PEG, polyvinylpyrrolidone (PVP; commercially known as Plasdone®), and the carbohydrate-based dispersing agents such as, for example, hydroxypropyl celluloses (e.g., HPC, HPC-SL, and HPC-L), hydroxypropyl methylcelluloses (e.g., HPMC K100, HPMC K4M, HPMC K15M, and HPMC K100M), carboxymethylcellulose sodium, methylcellulose, hydroxyethylcellulose, hydroxypropylcellulose, hydroxypropylmethylcellulose phthalate, hydroxypropylmethylcellulose acetate stearate (HPMCAS), noncrystalline cellulose, magnesium aluminum silicate, triethanolamine, polyvinyl alcohol (PVA), vinyl pyrrolidone/vinyl acetate copolymer (S630), 4-(1,1,3,3-tetramethylbutyl)-phenol polymer with ethylene oxide and formaldehyde (also known as tyloxapol), poloxamers (e.g., Pluronics F68®, F88®, and F108®, which are block copolymers of ethylene oxide and propylene oxide); and poloxamines (e.g., Tetronic 908®, also known as Poloxamine 908®, which is a tetrafunctional block copolymer derived from sequential addition of propylene oxide and ethylene oxide to ethylenediamine (BASF Corporation, Parsippany, N.J.)), polyvinylpyrrolidone K12, polyvinylpyrrolidone K17, polyvinylpyrrolidone K25, or polyvinylpyrrolidone K30, polyvinylpyrrolidone/vinyl acetate copolymer (S-630), polyethylene glycol, e.g., the polyethylene glycol can have a molecular weight of about 300 to about 6000, or about 3350 to about 4000, or about 7000 to about 5400, sodium carboxymethylcellulose, methylcellulose, polysorbate-80, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, polysorbate-80, sodium alginate, polyethoxylated sorbitan monolaurate, polyethoxylated sorbitan monolaurate, povidone, carbomers, polyvinyl alcohol (PVA), alginates, chitosans and combinations thereof. Plasticizers such as cellulose or triethyl cellulose can also be used as dispersing agents. Dispersing agents particularly useful in liposomal dispersions and self-emulsifying dispersions are dimyristoyl phosphatidyl choline, natural phosphatidyl choline from eggs, natural phosphatidyl glycerol from eggs, cholesterol and isopropyl myristate.

Combinations of one or more erosion facilitator with one or more diffusion facilitator can also be used in the present compositions.

The term “diluent” refers to chemical compounds that are used to dilute the compound of interest prior to delivery. Diluents can also be used to stabilize compounds because they can provide a more stable environment. Salts dissolved in buffered solutions (which also can provide pH control or maintenance) are utilized as diluents in the art, including, but not limited to a phosphate buffered saline solution. In certain embodiments, diluents increase bulk of the composition to facilitate compression or create sufficient bulk for homogenous blend for capsule filling. Such compounds include e.g., lactose, starch, mannitol, sorbitol, dextrose, microcrystalline cellulose such as Avicel®; dibasic calcium phosphate, dicalcium phosphate dihydrate; tricalcium phosphate, calcium phosphate; anhydrous lactose, spray-dried lactose; pregelatinized starch, compressible sugar, such as Di-Pac® (Amstar); mannitol, hydroxypropylmethylcellulose, hydroxypropylmethylcellulose acetate stearate, sucrose-based diluents, confectioner's sugar; monobasic calcium sulfate monohydrate, calcium sulfate dihydrate; calcium lactate trihydrate, dextrates; hydrolyzed cereal solids, amylose; powdered cellulose, calcium carbonate; glycine, kaolin; mannitol, sodium chloride; inositol, bentonite, and the like.

The term “disintegrate” includes both the dissolution and dispersion of the dosage form when contacted with gastrointestinal fluid. “Disintegration agents or disintegrants” facilitate the breakup or disintegration of a substance. Examples of disintegration agents include a starch, e.g., a natural starch such as corn starch or potato starch, a pregelatinized starch such as National 1551 or Amijel®, or sodium starch glycolate such as Promogel® or Explotab®, a cellulose such as a wood product, methylcrystalline cellulose, e.g., Avicel®, Avicel® PH101, Avicel® PH102, Avicel® PH105, Elcema® P100, Emcocel®, Vivacel®, Ming Tia®, and Solka-Floc®, methylcellulose, croscarmellose, or a cross-linked cellulose, such as cross-linked sodium carboxymethylcellulose (Ac-Di-Sol®), cross-linked carboxymethylcellulose, or cross-linked croscarmellose, a cross-linked starch such as sodium starch glycolate, a cross-linked polymer such as crospovidone, a cross-linked polyvinylpyrrolidone, alginate such as alginic acid or a salt of alginic acid such as sodium alginate, a clay such as Veegum® HV (magnesium aluminum silicate), a gum such as agar, guar, locust bean, Karaya, pectin, or tragacanth, sodium starch glycolate, bentonite, a natural sponge, a surfactant, a resin such as a cation-exchange resin, citrus pulp, sodium lauryl sulfate, sodium lauryl sulfate in combination starch, and the like.

“Drug absorption” or “absorption” typically refers to the process of movement of drug from site of administration of a drug across a barrier into a blood vessel or the site of action, e.g., a drug moving from the gastrointestinal tract into the portal vein or lymphatic system.

An “enteric coating” is a substance that remains substantially intact in the stomach but dissolves and releases the drug in the small intestine or colon. Generally, the enteric coating comprises a polymeric material that prevents release in the low pH environment of the stomach but that ionizes at a higher pH, typically a pH of 6 to 7, and thus dissolves sufficiently in the small intestine or colon to release the active agent therein.

“Erosion facilitators” include materials that control the erosion of a particular material in gastrointestinal fluid. Erosion facilitators are generally known to those of ordinary skill in the art. Exemplary erosion facilitators include, e.g., hydrophilic polymers, electrolytes, proteins, peptides, and amino acids.

“Filling agents” include compounds such as lactose, calcium carbonate, calcium phosphate, dibasic calcium phosphate, calcium sulfate, microcrystalline cellulose, cellulose powder, dextrose, dextrates, dextran, starches, pregelatinized starch, sucrose, xylitol, lactitol, mannitol, sorbitol, sodium chloride, polyethylene glycol, and the like.

“Flavoring agents” and/or “sweeteners” useful in the formulations described herein, include, e.g., acacia syrup, acesulfame K, alitame, anise, apple, aspartame, banana, Bavarian cream, berry, black currant, butterscotch, calcium citrate, camphor, caramel, cherry, cherry cream, chocolate, cinnamon, bubble gum, citrus, citrus punch, citrus cream, cotton candy, cocoa, cola, cool cherry, cool citrus, cyclamate, cylamate, dextrose, eucalyptus, eugenol, fructose, fruit punch, ginger, glycyrrhetinate, glycyrrhiza (licorice) syrup, grape, grapefruit, honey, isomalt, lemon, lime, lemon cream, monoammonium glyrrhizinate (MagnaSweet®), maltol, mannitol, maple, marshmallow, menthol, mint cream, mixed berry, neohesperidine DC, neotame, orange, pear, peach, peppermint, peppermint cream, Prosweet® Powder, raspberry, root beer, rum, saccharin, safrole, sorbitol, spearmint, spearmint cream, strawberry, strawberry cream, stevia, sucralose, sucrose, sodium saccharin, saccharin, aspartame, acesulfame potassium, mannitol, talin, sylitol, sucralose, sorbitol, Swiss cream, tagatose, tangerine, thaumatin, tutti fruitti, vanilla, walnut, watermelon, wild cherry, wintergreen, xylitol, or any combination of these flavoring ingredients, e.g., anise-menthol, cherry-anise, cinnamon-orange, cherry-cinnamon, chocolate-mint, honey-lemon, lemon-lime, lemon-mint, menthol-eucalyptus, orange-cream, vanilla-mint, and mixtures thereof.

“Lubricants” and “glidants” are compounds that prevent, reduce or inhibit adhesion or friction of materials. Exemplary lubricants include, e.g., stearic acid, calcium hydroxide, talc, sodium stearyl fumerate, a hydrocarbon such as mineral oil, or hydrogenated vegetable oil such as hydrogenated soybean oil (Sterotex®), higher fatty acids and their alkali-metal and alkaline earth metal salts, such as aluminum, calcium, magnesium, zinc, stearic acid, sodium stearates, glycerol, talc, waxes, Stearowet®, boric acid, sodium benzoate, sodium acetate, sodium chloride, leucine, a polyethylene glycol (e.g., PEG-4000) or a methoxypolyethylene glycol such as Carbowax™, sodium oleate, sodium benzoate, glyceryl behenate, polyethylene glycol, magnesium or sodium lauryl sulfate, colloidal silica such as Syloid™, Cab-O-Sil®, a starch such as corn starch, silicone oil, a surfactant, and the like.

A “measurable serum concentration” or “measurable plasma concentration” describes the blood serum or blood plasma concentration, typically measured in mg, μg, or ng of therapeutic agent per mL, dL, or L of blood serum, absorbed into the bloodstream after administration. As used herein, measurable plasma concentrations are typically measured in ng/ml or μg/ml.

“Pharmacodynamics” refers to the factors which determine the biologic response observed relative to the concentration of drug at a site of action.

“Pharmacokinetics” refers to the factors which determine the attainment and maintenance of the appropriate concentration of drug at a site of action.

“Plasticizers” are compounds used to soften the microencapsulation material or film coatings to make them less brittle. Suitable plasticizers include, e.g., polyethylene glycols such as PEG 300, PEG 400, PEG 600, PEG 1450, PEG 3350, and PEG 800, stearic acid, propylene glycol, oleic acid, triethyl cellulose and triacetin. In some embodiments, plasticizers can also function as dispersing agents or wetting agents.

“Solubilizers” include compounds such as triacetin, triethylcitrate, ethyl oleate, ethyl caprylate, sodium lauryl sulfate, sodium doccusate, vitamin E TPGS, dimethylacetamide, N-methylpyrrolidone, N-hydroxyethylpyrrolidone, polyvinylpyrrolidone, hydroxypropylmethyl cellulose, hydroxypropyl cyclodextrins, ethanol, n-butanol, isopropyl alcohol, cholesterol, bile salts, polyethylene glycol 200-600, glycofurol, transcutol, propylene glycol, and dimethyl isosorbide and the like.

“Stabilizers” include compounds such as any antioxidation agents, buffers, acids, preservatives and the like.

“Steady state,” as used herein, is when the amount of drug administered is equal to the amount of drug eliminated within one dosing interval resulting in a plateau or constant plasma drug exposure.

“Suspending agents” include compounds such as polyvinylpyrrolidone, e.g., polyvinylpyrrolidone K12, polyvinylpyrrolidone K17, polyvinylpyrrolidone K25, or polyvinylpyrrolidone K30, vinyl pyrrolidone/vinyl acetate copolymer (S630), polyethylene glycol, e.g., the polyethylene glycol can have a molecular weight of about 300 to about 6000, or about 3350 to about 4000, or about 7000 to about 5400, sodium carboxymethylcellulose, methylcellulose, hydroxypropylmethylcellulose, hydroxymethylcellulose acetate stearate, polysorbate-80, hydroxyethylcellulose, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, hydroxypropylmethylcellulose, hydroxyethylcellulose, polysorbate-80, sodium alginate, polyethoxylated sorbitan monolaurate, polyethoxylated sorbitan monolaurate, povidone and the like.

“Surfactants” include compounds such as sodium lauryl sulfate, sodium docusate, Tween 60 or 80, triacetin, vitamin E TPGS, sorbitan monooleate, polyoxyethylene sorbitan monooleate, polysorbates, polaxomers, bile salts, glyceryl monostearate, copolymers of ethylene oxide and propylene oxide, e.g., Pluronic® (BASF), and the like. Some other surfactants include polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40. In some embodiments, surfactants are included to enhance physical stability or for other purposes.

“Viscosity enhancing agents” include, e.g., methyl cellulose, xanthan gum, carboxymethyl cellulose, hydroxypropyl cellulose, hydroxypropylmethyl cellulose, hydroxypropylmethyl cellulose acetate stearate, hydroxypropylmethyl cellulose phthalate, carbomer, polyvinyl alcohol, alginates, acacia, chitosans and combinations thereof.

“Wetting agents” include compounds such as oleic acid, glyceryl monostearate, sorbitan monooleate, sorbitan monolaurate, triethanolamine oleate, polyoxyethylene sorbitan monooleate, polyoxyethylene sorbitan monolaurate, sodium docusate, sodium oleate, sodium lauryl sulfate, sodium doccusate, triacetin, Tween 80, vitamin E TPGS, ammonium salts and the like.

### Dosage Forms

In certain embodiments, described herein are dosage forms which comprise a Tec inhibitor. In some instances, the Tec inhibitor includes a Btk inhibitor or an ITK inhibitor. In some embodiments, the Btk inhibitor is a covalent Btk inhibitor. In some embodiments, described herein are dosage forms which comprise a covalent Btk inhibitor, (e.g. an irreversible covalent Btk inhibitor, e.g., ibrutinib).

In some embodiments, the pharmaceutical compositions are formulated such that the amount of the covalent Btk inhibitor (e.g., an irreversible covalent Btk inhibitor, e.g., ibrutinib) in each unit dosage form is between about 10 mg to about 1000 mg. In some embodiments, the pharmaceutical compositions are formulated such that the amount of the covalent Btk inhibitor (e.g., an irreversible covalent Btk inhibitor, e.g., ibrutinib) in each unit dosage form is about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 250 mg, about 300 mg, about 350 mg, about 400 mg, about 450 mg, about 500 mg, about 550 mg, about 600 mg, about 700 mg or about 800 mg. In some embodiments, the therapeutically-effective amount of Ibrutinib is between about 40 mg and about 140 mg. In some embodiments, the pharmaceutical compositions are formulated such that the amount of the covalent Btk inhibitor (e.g., an irreversible covalent Btk inhibitor, e.g., ibrutinib) in each unit dosage form is about 140 mg per.

In some instances, the pharmaceutical formulations described herein are administered by any suitable administration route, including but not limited to, oral, parenteral (e.g., intravenous, subcutaneous, intramuscular), intranasal, buccal, topical, rectal, or transdermal administration routes.

In some instances, the pharmaceutical compositions described herein are formulated into any suitable dosage form, including but not limited to, aqueous oral dispersions, liquids, gels, syrups, elixirs, slurries, suspensions and the like, for oral ingestion by an individual to be treated, solid oral dosage forms, aerosols, controlled release formulations, fast melt formulations, effervescent formulations, lyophilized formulations, tablets, powders, pills, dragees, capsules, delayed release formulations, extended release formulations, pulsatile release formulations, multiparticulate formulations, and mixed immediate release and controlled release formulations. In some embodiments, the compositions are formulated into capsules. In some embodiments, the compositions are formulated into solutions (for example, for IV administration).

Pharmaceutical preparations for oral use can be obtained by mixing one or more solid excipient with one or more of the compounds described herein, optionally grinding the resulting mixture, and processing the mixture of granules, after adding suitable auxiliaries, if desired, to obtain tablets or dragee cores. Suitable excipients include, for example, fillers such as sugars, including lactose, sucrose, mannitol, or sorbitol; cellulose preparations such as, for example, maize starch, wheat starch, rice starch, potato starch, gelatin, gum tragacanth, methylcellulose, microcrystalline cellulose, hydroxypropylmethylcellulose, sodium carboxymethylcellulose; or others such as: polyvinylpyrrolidone (PVP or povidone) or calcium phosphate. In some embodiments, disintegrating agents are added, such as the cross-linked croscarmellose sodium, polyvinylpyrrolidone, agar, or alginic acid or a salt thereof such as sodium alginate.

Dragee cores are provided with suitable coatings. For this purpose, in some embodiments, concentrated sugar solutions are used, which, in particular embodiments, optionally contain gum arabic, talc, polyvinylpyrrolidone, carbopol gel, polyethylene glycol, and/or titanium dioxide, lacquer solutions, and suitable organic solvents or solvent mixtures. In some embodiments, dyestuffs or pigments are added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In some embodiments, in soft capsules, the active compounds are dissolved or suspended in suitable liquids, such as fatty oils, liquid paraffin, or liquid polyethylene glycols. In addition, in some embodiments, stabilizers are added. All formulations for oral administration should be in dosages suitable for such administration.

In some embodiments, the solid dosage forms disclosed herein are in the form of a tablet, (including a suspension tablet, a fast-melt tablet, a bite-disintegration tablet, a rapid-disintegration tablet, an effervescent tablet, or a caplet), a pill, a powder (including a sterile packaged powder, a dispensable powder, or an effervescent powder) a capsule (including both soft or hard capsules, e.g., capsules made from animal-derived gelatin or plant-derived HPMC, or “sprinkle capsules”), solid dispersion, solid solution, bioerodible dosage form, controlled release formulations, pulsatile release dosage forms, multiparticulate dosage forms, pellets, granules, or an aerosol. In other embodiments, the pharmaceutical formulation is in the form of a powder. In still other embodiments, the pharmaceutical formulation is in the form of a tablet, including but not limited to, a fast-melt tablet. Additionally, in some embodiments, pharmaceutical formulations described herein are administered as a single capsule or in multiple capsule dosage form. In some embodiments, the pharmaceutical formulation is administered in two, or three, or four, capsules or tablets.

In some embodiments, solid dosage forms, e.g., tablets, effervescent tablets, and capsules, are prepared by mixing particles of ibrutinib, with one or more pharmaceutical excipients to form a bulk blend composition. When referring to these bulk blend compositions as homogeneous, it is meant that the particles of ibrutinib are dispersed evenly throughout the composition so that the composition can be readily subdivided into equally effective unit dosage forms, such as tablets, pills, and capsules. In some embodiments, the individual unit dosages also include film coatings, which disintegrate upon oral ingestion or upon contact with diluent. These formulations can be manufactured by conventional pharmacological techniques.

Conventional pharmacological techniques include, e.g., one or a combination of methods: (1) dry mixing, (2) direct compression, (3) milling, (4) dry or non-aqueous granulation, (5) wet granulation, or (6) fusion. See, e.g., Lachman et al., The Theory and Practice of Industrial Pharmacy (1986). Other methods include, e.g., spray drying, pan coating, melt granulation, granulation, fluidized bed spray drying or coating (e.g., wurster coating), tangential coating, top spraying, tableting, extruding and the like.

The pharmaceutical solid dosage forms described herein can include a compound described herein and one or more pharmaceutically acceptable additives such as a compatible carrier, binder, filling agent, suspending agent, flavoring agent, sweetening agent, disintegrating agent, dispersing agent, surfactant, lubricant, colorant, diluent, solubilizer, moistening agent, plasticizer, stabilizer, penetration enhancer, wetting agent, anti-foaming agent, antioxidant, preservative, or one or more combination thereof. In still other aspects, using standard coating procedures, such as those described in Remington's Pharmaceutical Sciences, 20th Edition (2000), a film coating is provided around the formulation of ibrutinib. In another embodiment, some or all of the particles of ibrutinib, are not microencapsulated and are uncoated.

Suitable carriers for use in the solid dosage forms described herein include, but are not limited to, acacia, gelatin, colloidal silicon dioxide, calcium glycerophosphate, calcium lactate, maltodextrin, glycerine, magnesium silicate, sodium caseinate, soy lecithin, sodium chloride, tricalcium phosphate, dipotassium phosphate, sodium stearoyl lactylate, carrageenan, monoglyceride, diglyceride, pregelatinized starch, hydroxypropylmethylcellulose, hydroxypropylmethylcellulose acetate stearate, sucrose, microcrystalline cellulose, lactose, mannitol and the like.

Suitable filling agents for use in the solid dosage forms described herein include, but are not limited to, lactose, calcium carbonate, calcium phosphate, dibasic calcium phosphate, calcium sulfate, microcrystalline cellulose, cellulose powder, dextrose, dextrates, dextran, starches, pregelatinized starch, hydroxypropylmethycellulose (HPMC), hydroxypropylmethycellulose phthalate, hydroxypropylmethylcellulose acetate stearate (HPMCAS), sucrose, xylitol, lactitol, mannitol, sorbitol, sodium chloride, polyethylene glycol, and the like.

In order to release the compound of one or more of the therapeutic agents described herein, from a solid dosage form matrix as efficiently as possible, disintegrants are often used in the formulation, especially when the dosage forms are compressed with binder. Disintegrants help rupturing the dosage form matrix by swelling or capillary action when moisture is absorbed into the dosage form. Suitable disintegrants for use in the solid dosage forms described herein include, but are not limited to, natural starch such as corn starch or potato starch, a pregelatinized starch such as National 1551 or Amijel®, or sodium starch glycolate such as Promogel® or Explotab®, a cellulose such as a wood product, methylcrystalline cellulose, e.g., Avicel®, Avicel® PH101, Avicel® PH102, Avicel® PH105, Elcema® P100, Emcocel®, Vivacel®, Ming Tia®, and Solka-Floc®, methylcellulose, croscarmellose, or a cross-linked cellulose, such as cross-linked sodium carboxymethylcellulose (Ac-Di-Sol®), cross-linked carboxymethylcellulose, or cross-linked croscarmellose, a cross-linked starch such as sodium starch glycolate, a cross-linked polymer such as crospovidone, a cross-linked polyvinylpyrrolidone, alginate such as alginic acid or a salt of alginic acid such as sodium alginate, a clay such as Veegum® HV (magnesium aluminum silicate), a gum such as agar, guar, locust bean, Karaya, pectin, or tragacanth, sodium starch glycolate, bentonite, a natural sponge, a surfactant, a resin such as a cation-exchange resin, citrus pulp, sodium lauryl sulfate, sodium lauryl sulfate in combination starch, and the like.

Binders impart cohesiveness to solid oral dosage form formulations: for powder filled capsule formulation, they aid in plug formation that can be filled into soft or hard shell capsules and for tablet formulation, they ensure the tablet remaining intact after compression and help assure blend uniformity prior to a compression or fill step. Materials suitable for use as binders in the solid dosage forms described herein include, but are not limited to, carboxymethylcellulose, methylcellulose (e.g., Methocel®), hydroxypropylmethylcellulose (e.g. Hypromellose USP Pharmacoat-603, hydroxypropylmethylcellulose acetate stearate (Aqoate HS-LF and HS), hydroxyethylcellulose, hydroxypropylcellulose (e.g., Klucer), ethylcellulose (e.g., Ethocel®), and microcrystalline cellulose (e.g., Avicel®), microcrystalline dextrose, amylose, magnesium aluminum silicate, polysaccharide acids, bentonites, gelatin, polyvinylpyrrolidone/vinyl acetate copolymer, crospovidone, povidone, starch, pregelatinized starch, tragacanth, dextrin, a sugar, such as sucrose (e.g., Dipac®), glucose, dextrose, molasses, mannitol, sorbitol, xylitol (e.g., Xylitab®), lactose, a natural or synthetic gum such as acacia, tragacanth, ghatti gum, mucilage of isapol husks, starch, polyvinylpyrrolidone (e.g., Povidone® CL, Kollidon® CL, Polyplasdone® XL-10, and Povidone® K-12), larch arabogalactan, Veegum®, polyethylene glycol, waxes, sodium alginate, and the like.

In general, binder levels of 20-70% are used in powder-filled gelatin capsule formulations. Binder usage level in tablet formulations varies whether direct compression, wet granulation, roller compaction, or usage of other excipients such as fillers which itself can act as moderate binder. Formulators skilled in art can determine the binder level for the formulations, but binder usage level of up to 70% in tablet formulations is common.

Suitable lubricants or glidants for use in the solid dosage forms described herein include, but are not limited to, stearic acid, calcium hydroxide, talc, corn starch, sodium stearyl fumerate, alkali-metal and alkaline earth metal salts, such as aluminum, calcium, magnesium, zinc, stearic acid, sodium stearates, magnesium stearate, zinc stearate, waxes, Stearowet®, boric acid, sodium benzoate, sodium acetate, sodium chloride, leucine, a polyethylene glycol or a methoxypolyethylene glycol such as Carbowax™, PEG 4000, PEG 5000, PEG 6000, propylene glycol, sodium oleate, glyceryl behenate, glyceryl palmitostearate, glyceryl benzoate, magnesium or sodium lauryl sulfate, and the like.

Suitable diluents for use in the solid dosage forms described herein include, but are not limited to, sugars (including lactose, sucrose, and dextrose), polysaccharides (including dextrates and maltodextrin), polyols (including mannitol, xylitol, and sorbitol), cyclodextrins and the like.

The term “non water-soluble diluent” represents compounds typically used in the formulation of pharmaceuticals, such as calcium phosphate, calcium sulfate, starches, modified starches and microcrystalline cellulose, and microcellulose (e.g., having a density of about 0.45 g/cm³, e.g. Avicel, powdered cellulose), and talc.

Suitable wetting agents for use in the solid dosage forms described herein include, for example, oleic acid, glyceryl monostearate, sorbitan monooleate, sorbitan monolaurate, triethanolamine oleate, polyoxyethylene sorbitan monooleate, polyoxyethylene sorbitan monolaurate, quaternary ammonium compounds (e.g., Polyquat 10®), sodium oleate, sodium lauryl sulfate, magnesium stearate, sodium docusate, triacetin, vitamin E TPGS and the like.

Suitable surfactants for use in the solid dosage forms described herein include, for example, sodium lauryl sulfate, sorbitan monooleate, polyoxyethylene sorbitan monooleate, polysorbates, polaxomers, bile salts, glyceryl monostearate, copolymers of ethylene oxide and propylene oxide, e.g., Pluronic® (BASF), and the like.

Suitable suspending agents for use in the solid dosage forms described here include, but are not limited to, polyvinylpyrrolidone, e.g., polyvinylpyrrolidone K12, polyvinylpyrrolidone K17, polyvinylpyrrolidone K25, or polyvinylpyrrolidone K30, polyethylene glycol, e.g., the polyethylene glycol can have a molecular weight of about 300 to about 6000, or about 3350 to about 4000, or about 7000 to about 5400, vinyl pyrrolidone/vinyl acetate copolymer (S630), sodium carboxymethylcellulose, methylcellulose, hydroxy-propylmethylcellulose, polysorbate-80, hydroxyethylcellulose, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, hydroxypropylmethylcellulose, hydroxyethylcellulose, polysorbate-80, sodium alginate, polyethoxylated sorbitan monolaurate, polyethoxylated sorbitan monolaurate, povidone and the like.

Suitable antioxidants for use in the solid dosage forms described herein include, for example, e.g., butylated hydroxytoluene (BHT), sodium ascorbate, and tocopherol.

It should be appreciated that there is considerable overlap between additives used in the solid dosage forms described herein. Thus, the above-listed additives should be taken as merely exemplary, and not limiting, of the types of additives that can be included in solid dosage forms described herein. The amounts of such additives can be readily determined by one skilled in the art, according to the particular properties desired.

In other embodiments, one or more layers of the pharmaceutical formulation are plasticized. Illustratively, a plasticizer is generally a high boiling point solid or liquid. Suitable plasticizers can be added from about 0.01% to about 50% by weight (w/w) of the coating composition. Plasticizers include, but are not limited to, diethyl phthalate, citrate esters, polyethylene glycol, glycerol, acetylated glycerides, triacetin, polypropylene glycol, polyethylene glycol, triethyl citrate, dibutyl sebacate, stearic acid, stearol, stearate, and castor oil.

Compressed tablets are solid dosage forms prepared by compacting the bulk blend of the formulations described above. In various embodiments, compressed tablets which are designed to dissolve in the mouth will include one or more flavoring agents. In other embodiments, the compressed tablets will include a film surrounding the final compressed tablet.

In some embodiments, the film coating can provide a delayed release of ibrutinib or the second agent, from the formulation. In other embodiments, the film coating aids in patient compliance (e.g., Opadry® coatings or sugar coating). Film coatings including Opadry® typically range from about 1% to about 3% of the tablet weight. In other embodiments, the compressed tablets include one or more excipients.

In some embodiments, a capsule is prepared, for example, by placing the bulk blend of the formulation of ibrutinib or the second agent, described above, inside of a capsule. In some embodiments, the formulations (non-aqueous suspensions and solutions) are placed in a soft gelatin capsule. In other embodiments, the formulations are placed in standard gelatin capsules or non-gelatin capsules such as capsules comprising HPMC. In other embodiments, the formulation is placed in a sprinkle capsule, wherein the capsule can be swallowed whole or the capsule can be opened and the contents sprinkled on food prior to eating. In some embodiments, the therapeutic dose is split into multiple (e.g., two, three, or four) capsules. In some embodiments, the entire dose of the formulation is delivered in a capsule form.

In various embodiments, the particles of ibrutinib, and one or more excipients are dry blended and compressed into a mass, such as a tablet, having a hardness sufficient to provide a pharmaceutical composition that substantially disintegrates within less than about 30 minutes, less than about 35 minutes, less than about 40 minutes, less than about 45 minutes, less than about 50 minutes, less than about 55 minutes, or less than about 60 minutes, after oral administration, thereby releasing the formulation into the gastrointestinal fluid.

In another aspect, in some embodiments, dosage forms include microencapsulated formulations. In some embodiments, one or more other compatible materials are present in the microencapsulation material. Exemplary materials include, but are not limited to, pH modifiers, erosion facilitators, anti-foaming agents, antioxidants, flavoring agents, and carrier materials such as binders, suspending agents, disintegration agents, filling agents, surfactants, solubilizers, stabilizers, lubricants, wetting agents, and diluents.

Materials useful for the microencapsulation described herein include materials compatible with ibrutinib, which sufficiently isolate the compound of any of ibrutinib, from other non-compatible excipients. Materials compatible with compounds of any of ibrutinib, are those that delay the release of the compounds of any of ibrutinib, in vivo.

Exemplary microencapsulation materials useful for delaying the release of the formulations including compounds described herein, include, but are not limited to, hydroxypropyl cellulose ethers (HPC) such as Klucel® or Nisso HPC, low-substituted hydroxypropyl cellulose ethers (L-HPC), hydroxypropyl methyl cellulose ethers (HPMC) such as Seppifilm-LC, Pharmacoat®, Metolose SR, Methocel®-E, Opadry YS, PrimaFlo, Benecel MP824, and Benecel MP843, methylcellulose polymers such as Methocel®-A, hydroxypropylmethylcellulose acetate stearate Aqoat (HF-LS, HF-LG,HF-MS) and Metolose®, Ethylcelluloses (EC) and mixtures thereof such as E461, Ethocel®, Aqualon®-EC, Surelease®, Polyvinyl alcohol (PVA) such as Opadry AMB, hydroxyethylcelluloses such as Natrosol®, carboxymethylcelluloses and salts of carboxymethylcelluloses (CMC) such as Aqualon®-CMC, polyvinyl alcohol and polyethylene glycol co-polymers such as Kollicoat IR®, monoglycerides (Myverol), triglycerides (KLX), polyethylene glycols, modified food starch, acrylic polymers and mixtures of acrylic polymers with cellulose ethers such as Eudragit® EPO, Eudragit® L30D-55, Eudragit® FS 30D Eudragit® L100-55, Eudragit® L100, Eudragit® 5100, Eudragit® RD100, Eudragit® E100, Eudragit® L12.5, Eudragit® 512.5, Eudragit® NE30D, and Eudragit® NE 40D, cellulose acetate phthalate, sepifilms such as mixtures of HPMC and stearic acid, cyclodextrins, and mixtures of these materials.

In still other embodiments, plasticizers such as polyethylene glycols, e.g., PEG 300, PEG 400, PEG 600, PEG 1450, PEG 3350, and PEG 800, stearic acid, propylene glycol, oleic acid, and triacetin are incorporated into the microencapsulation material. In other embodiments, the microencapsulating material useful for delaying the release of the pharmaceutical compositions is from the USP or the National Formulary (NF). In yet other embodiments, the microencapsulation material is Klucel. In still other embodiments, the microencapsulation material is methocel.

In some embodiments, microencapsulated compounds of any of ibrutinib, are formulated by methods known by one of ordinary skill in the art. Such known methods include, e.g., spray drying processes, spinning disk-solvent processes, hot melt processes, spray chilling methods, fluidized bed, electrostatic deposition, centrifugal extrusion, rotational suspension separation, polymerization at liquid-gas or solid-gas interface, pressure extrusion, or spraying solvent extraction bath. In addition to these, several chemical techniques, e.g., complex coacervation, solvent evaporation, polymer-polymer incompatibility, interfacial polymerization in liquid media, in situ polymerization, in-liquid drying, and desolvation in liquid media could also be used. Furthermore, in some embodiments, other methods such as roller compaction, extrusion/spheronization, coacervation, or nanoparticle coating are used.

In one embodiment, the particles of compounds of any of ibrutinib, are microencapsulated prior to being formulated into one of the above forms. In still another embodiment, some or most of the particles are coated prior to being further formulated by using standard coating procedures, such as those described in Remington's Pharmaceutical Sciences, 20th Edition (2000).

In other embodiments, the solid dosage formulations of the compounds of any of ibrutinib, are plasticized (coated) with one or more layers. Illustratively, a plasticizer is generally a high boiling point solid or liquid. Suitable plasticizers can be added from about 0.01% to about 50% by weight (w/w) of the coating composition. Plasticizers include, but are not limited to, diethyl phthalate, citrate esters, polyethylene glycol, glycerol, acetylated glycerides, triacetin, polypropylene glycol, polyethylene glycol, triethyl citrate, dibutyl sebacate, stearic acid, stearol, stearate, and castor oil.

In other embodiments, a powder including the formulations with a compound of any of ibrutinib, described herein, is formulated to include one or more pharmaceutical excipients and flavors. In some embodiments, such a powder is prepared, for example, by mixing the formulation and optional pharmaceutical excipients to form a bulk blend composition. Additional embodiments also include a suspending agent and/or a wetting agent. This bulk blend is uniformly subdivided into unit dosage packaging or multi-dosage packaging units.

In still other embodiments, effervescent powders are also prepared in accordance with the present disclosure. Effervescent salts have been used to disperse medicines in water for oral administration. Effervescent salts are granules or coarse powders containing a medicinal agent in a dry mixture, usually composed of sodium bicarbonate, citric acid and/or tartaric acid. When salts of the compositions described herein are added to water, the acids and the base react to liberate carbon dioxide gas, thereby causing “effervescence.” Examples of effervescent salts include, e.g., the following ingredients: sodium bicarbonate or a mixture of sodium bicarbonate and sodium carbonate, citric acid and/or tartaric acid. Any acid-base combination that results in the liberation of carbon dioxide can be used in place of the combination of sodium bicarbonate and citric and tartaric acids, as long as the ingredients were suitable for pharmaceutical use and result in a pH of about 6.0 or higher.

In some embodiments, the solid dosage forms described herein can be formulated as enteric coated delayed release oral dosage forms, i.e., as an oral dosage form of a pharmaceutical composition as described herein which utilizes an enteric coating to affect release in the small intestine of the gastrointestinal tract. In some embodiments, the enteric coated dosage form is a compressed or molded or extruded tablet/mold (coated or uncoated) containing granules, powder, pellets, beads or particles of the active ingredient and/or other composition components, which are themselves coated or uncoated. In some embodiments, the enteric coated oral dosage form is a capsule (coated or uncoated) containing pellets, beads or granules of the solid carrier or the composition, which are themselves coated or uncoated.

The term “delayed release” as used herein refers to the delivery so that the release can be accomplished at some generally predictable location in the intestinal tract more distal to that which would have been accomplished if there had been no delayed release alterations. In some embodiments the method for delay of release is coating. Any coatings should be applied to a sufficient thickness such that the entire coating does not dissolve in the gastrointestinal fluids at pH below about 5, but does dissolve at pH about 5 and above. It is expected that any anionic polymer exhibiting a pH-dependent solubility profile can be used as an enteric coating in the methods and compositions described herein to achieve delivery to the lower gastrointestinal tract. In some embodiments the polymers described herein are anionic carboxylic polymers. In other embodiments, the polymers and compatible mixtures thereof, and some of their properties, include, but are not limited to:

Shellac, also called purified lac, a refined product obtained from the resinous secretion of an insect. This coating dissolves in media of pH >7;

Acrylic polymers. The performance of acrylic polymers (primarily their solubility in biological fluids) can vary based on the degree and type of substitution. Examples of suitable acrylic polymers include methacrylic acid copolymers and ammonium methacrylate copolymers.

The Eudragit series E, L, S, RL, RS and NE (Rohm Pharma) are available as solubilized in organic solvent, aqueous dispersion, or dry powders. The Eudragit series RL, NE, and RS are insoluble in the gastrointestinal tract but are permeable and are used primarily for colonic targeting. The Eudragit series E dissolve in the stomach. The Eudragit series L, L-30D and S are insoluble in stomach and dissolve in the intestine;

Cellulose Derivatives. Examples of suitable cellulose derivatives are: ethyl cellulose; reaction mixtures of partial acetate esters of cellulose with phthalic anhydride. The performance can vary based on the degree and type of substitution. Cellulose acetate phthalate (CAP) dissolves in pH >6. Aquateric (FMC) is an aqueous based system and is a spray dried CAP psuedolatex with particles <1 μm. Other components in Aquateric can include pluronics, Tweens, and acetylated monoglycerides. Other suitable cellulose derivatives include: cellulose acetate trimellitate (Eastman); methylcellulose (Pharmacoat, Methocel); hydroxypropylmethyl cellulose phthalate (HPMCP); hydroxypropylmethyl cellulose succinate (HPMCS); and hydroxypropylmethylcellulose acetate succinate (e.g., AQOAT (Shin Etsu)). The performance can vary based on the degree and type of substitution. For example, HPMCP such as, HP-50, HP-55, HP-555, HP-55F grades are suitable. The performance can vary based on the degree and type of substitution. For example, suitable grades of hydroxypropylmethylcellulose acetate succinate include, but are not limited to, AS-LG (LF), which dissolves at pH 5, AS-MG (MF), which dissolves at pH 5.5, and AS-HG (HF), which dissolves at higher pH. These polymers are offered as granules, or as fine powders for aqueous dispersions; Poly Vinyl Acetate Phthalate (PVAP). PVAP dissolves in pH >5, and it is much less permeable to water vapor and gastric fluids.

In some embodiments, the coating can, and usually does, contain a plasticizer and possibly other coating excipients such as colorants, talc, and/or magnesium stearate, which are well known in the art. Suitable plasticizers include triethyl citrate (Citroflex 2), triacetin (glyceryl triacetate), acetyl triethyl citrate (Citroflec A2), Carbowax 400 (polyethylene glycol 400), diethyl phthalate, tributyl citrate, acetylated monoglycerides, glycerol, fatty acid esters, propylene glycol, and dibutyl phthalate. In particular, anionic carboxylic acrylic polymers usually will contain 10-25% by weight of a plasticizer, especially dibutyl phthalate, polyethylene glycol, triethyl citrate and triacetin. Conventional coating techniques such as spray or pan coating are employed to apply coatings. The coating thickness must be sufficient to ensure that the oral dosage form remains intact until the desired site of topical delivery in the intestinal tract is reached.

In some embodiments, colorants, detackifiers, surfactants, antifoaming agents, lubricants (e.g., carnuba wax or PEG) are added to the coatings besides plasticizers to solubilize or disperse the coating material, and to improve coating performance and the coated product.

In other embodiments, the formulations described herein, which include ibrutinib, are delivered using a pulsatile dosage form. A pulsatile dosage form is capable of providing one or more immediate release pulses at predetermined time points after a controlled lag time or at specific sites. Many other types of controlled release systems known to those of ordinary skill in the art and are suitable for use with the formulations described herein. Examples of such delivery systems include, e.g., polymer-based systems, such as polylactic and polyglycolic acid, plyanhydrides and polycaprolactone; porous matrices, nonpolymer-based systems that are lipids, including sterols, such as cholesterol, cholesterol esters and fatty acids, or neutral fats, such as mono-, di- and triglycerides; hydrogel release systems; silastic systems; peptide-based systems; wax coatings, bioerodible dosage forms, compressed tablets using conventional binders and the like. See, e.g., Liberman et al., Pharmaceutical Dosage Forms, 2 Ed., Vol. 1, pp. 209-214 (1990); Singh et al., Encyclopedia of Pharmaceutical Technology, 2^{nd} Ed., pp. 751-753 (2002); U.S. Pat. Nos. 4,327,725, 4,624,848, 4,968,509, 5,461,140, 5,456,923, 5,516,527, 5,622,721, 5,686,105, 5,700,410, 5,977,175, 6,465,014 and 6,932,983.

In some embodiments, pharmaceutical formulations are provided that include particles of ibrutinib, described herein and at least one dispersing agent or suspending agent for oral administration to a subject. In some embodiments, the formulations are a powder and/or granules for suspension, and upon admixture with water, a substantially uniform suspension is obtained.

Liquid formulation dosage forms for oral administration can be aqueous suspensions selected from the group including, but not limited to, pharmaceutically acceptable aqueous oral dispersions, emulsions, solutions, elixirs, gels, and syrups. See, e.g., Singh et al., Encyclopedia of Pharmaceutical Technology, 2^{nd} Ed., pp. 754-757 (2002). In addition, in some embodiments, the liquid dosage forms include additives, such as: (a) disintegrating agents; (b) dispersing agents; (c) wetting agents; (d) at least one preservative, (e) viscosity enhancing agents, (f) at least one sweetening agent, and (g) at least one flavoring agent. In some embodiments, the aqueous dispersions can further include a crystalline inhibitor.

The aqueous suspensions and dispersions described herein can remain in a homogenous state, as defined in The USP Pharmacists' Pharmacopeia (2005 edition, chapter 905), for at least 4 hours. The homogeneity should be determined by a sampling method consistent with regard to determining homogeneity of the entire composition. In one embodiment, an aqueous suspension can be re-suspended into a homogenous suspension by physical agitation lasting less than 1 minute. In another embodiment, an aqueous suspension can be re-suspended into a homogenous suspension by physical agitation lasting less than 45 seconds. In yet another embodiment, an aqueous suspension can be re-suspended into a homogenous suspension by physical agitation lasting less than 30 seconds. In still another embodiment, no agitation is necessary to maintain a homogeneous aqueous dispersion.

Examples of disintegrating agents for use in the aqueous suspensions and dispersions include, but are not limited to, a starch, e.g., a natural starch such as corn starch or potato starch, a pregelatinized starch such as National 1551 or Amijel®, or sodium starch glycolate such as Promogel® or Explotab®; a cellulose such as a wood product, methylcrystalline cellulose, e.g., Avicel®, Avicel® PH101, Avicel® PH102, Avicel® PH105, Elcema® P100, Emcocel®, Vivacel®, Ming Tia®, and Solka-Floc®, methylcellulose, croscarmellose, or a cross-linked cellulose, such as cross-linked sodium carboxymethylcellulose (Ac-Di-Sol®), cross-linked carboxymethylcellulose, or cross-linked croscarmellose; a cross-linked starch such as sodium starch glycolate; a cross-linked polymer such as crospovidone; a cross-linked polyvinylpyrrolidone; alginate such as alginic acid or a salt of alginic acid such as sodium alginate; a clay such as Veegum® HV (magnesium aluminum silicate); a gum such as agar, guar, locust bean, Karaya, pectin, or tragacanth; sodium starch glycolate; bentonite; a natural sponge; a surfactant; a resin such as a cation-exchange resin; citrus pulp; sodium lauryl sulfate; sodium lauryl sulfate in combination starch; and the like.

In some embodiments, the dispersing agents suitable for the aqueous suspensions and dispersions described herein are known in the art and include, for example, hydrophilic polymers, electrolytes, Tween® 60 or 80, PEG, polyvinylpyrrolidone (PVP; commercially known as Plasdone®), and the carbohydrate-based dispersing agents such as, for example, hydroxypropylcellulose and hydroxypropyl cellulose ethers (e.g., HPC, HPC-SL, and HPC-L), hydroxypropyl methylcellulose and hydroxypropyl methylcellulose ethers (e.g. HPMC K100, HPMC K4M, HPMC K15M, and HPMC K100M), carboxymethylcellulose sodium, methylcellulose, hydroxyethylcellulose, hydroxypropylmethyl-cellulose phthalate, hydroxypropylmethyl-cellulose acetate stearate, noncrystalline cellulose, magnesium aluminum silicate, triethanolamine, polyvinyl alcohol (PVA), polyvinylpyrrolidone/vinyl acetate copolymer (Plasdone®, e.g., S-630), 4-(1,1,3,3-tetramethylbutyl)-phenol polymer with ethylene oxide and formaldehyde (also known as tyloxapol), poloxamers (e.g., Pluronics F68®, F88®, and F108®, which are block copolymers of ethylene oxide and propylene oxide); and poloxamines (e.g., Tetronic 908®, also known as Poloxamine 908®, which is a tetrafunctional block copolymer derived from sequential addition of propylene oxide and ethylene oxide to ethylenediamine (BASF Corporation, Parsippany, N.J.)). In other embodiments, the dispersing agent is selected from a group not comprising one of the following agents: hydrophilic polymers; electrolytes; Tween® 60 or 80; PEG; polyvinylpyrrolidone (PVP); hydroxypropylcellulose and hydroxypropyl cellulose ethers (e.g., HPC, HPC-SL, and HPC-L); hydroxypropyl methylcellulose and hydroxypropyl methylcellulose ethers (e.g. HPMC K100, HPMC K4M, HPMC K15M, HPMC K100M, and Pharmacoat® USP 2910 (Shin-Etsu)); carboxymethylcellulose sodium; methylcellulose; hydroxyethylcellulose; hydroxypropylmethyl-cellulose phthalate; hydroxypropylmethyl-cellulose acetate stearate; non-crystalline cellulose; magnesium aluminum silicate; triethanolamine; polyvinyl alcohol (PVA); 4-(1,1,3,3-tetramethylbutyl)-phenol polymer with ethylene oxide and formaldehyde; poloxamers (e.g., Pluronics F68®, F88®, and F108®, which are block copolymers of ethylene oxide and propylene oxide); or poloxamines (e.g., Tetronic 908®, also known as Poloxamine 908®).

Wetting agents suitable for the aqueous suspensions and dispersions described herein are known in the art and include, but are not limited to, cetyl alcohol, glycerol monostearate, polyoxyethylene sorbitan fatty acid esters (e.g., the commercially available Tweens® such as e.g., Tween 20® and Tween 80® (ICI Specialty Chemicals)), and polyethylene glycols (e.g., Carbowaxs 3350® and 1450®, and Carbopol 934® (Union Carbide)), oleic acid, glyceryl monostearate, sorbitan monooleate, sorbitan monolaurate, triethanolamine oleate, polyoxyethylene sorbitan monooleate, polyoxyethylene sorbitan monolaurate, sodium oleate, sodium lauryl sulfate, sodium docusate, triacetin, vitamin E TPGS, sodium taurocholate, simethicone, phosphotidylcholine and the like.

Suitable preservatives for the aqueous suspensions or dispersions described herein include, for example, potassium sorbate, parabens (e.g., methylparaben and propylparaben), benzoic acid and its salts, other esters of parahydroxybenzoic acid such as butylparaben, alcohols such as ethyl alcohol or benzyl alcohol, phenolic compounds such as phenol, or quaternary compounds such as benzalkonium chloride. Preservatives, as used herein, are incorporated into the dosage form at a concentration sufficient to inhibit microbial growth.

Suitable viscosity enhancing agents for the aqueous suspensions or dispersions described herein include, but are not limited to, methyl cellulose, xanthan gum, carboxymethyl cellulose, hydroxypropyl cellulose, hydroxypropylmethyl cellulose, Plasdon® S-630, carbomer, polyvinyl alcohol, alginates, acacia, chitosans and combinations thereof. The concentration of the viscosity enhancing agent will depend upon the agent selected and the viscosity desired.

Examples of sweetening agents suitable for the aqueous suspensions or dispersions described herein include, for example, acacia syrup, acesulfame K, alitame, anise, apple, aspartame, banana, Bavarian cream, berry, black currant, butterscotch, calcium citrate, camphor, caramel, cherry, cherry cream, chocolate, cinnamon, bubble gum, citrus, citrus punch, citrus cream, cotton candy, cocoa, cola, cool cherry, cool citrus, cyclamate, cylamate, dextrose, eucalyptus, eugenol, fructose, fruit punch, ginger, glycyrrhetinate, glycyrrhiza (licorice) syrup, grape, grapefruit, honey, isomalt, lemon, lime, lemon cream, monoammonium glyrrhizinate (MagnaSweet®), maltol, mannitol, maple, marshmallow, menthol, mint cream, mixed berry, neohesperidine DC, neotame, orange, pear, peach, peppermint, peppermint cream, Prosweet® Powder, raspberry, root beer, rum, saccharin, safrole, sorbitol, spearmint, spearmint cream, strawberry, strawberry cream, stevia, sucralose, sucrose, sodium saccharin, saccharin, aspartame, acesulfame potassium, mannitol, talin, sucralose, sorbitol, swiss cream, tagatose, tangerine, thaumatin, tutti fruitti, vanilla, walnut, watermelon, wild cherry, wintergreen, xylitol, or any combination of these flavoring ingredients, e.g., anise-menthol, cherry-anise, cinnamon-orange, cherry-cinnamon, chocolate-mint, honey-lemon, lemon-lime, lemon-mint, menthol-eucalyptus, orange-cream, vanilla-mint, and mixtures thereof. In one embodiment, the aqueous liquid dispersion can comprise a sweetening agent or flavoring agent in a concentration ranging from about 0.001% to about 1.0% the volume of the aqueous dispersion. In another embodiment, the aqueous liquid dispersion can comprise a sweetening agent or flavoring agent in a concentration ranging from about 0.005% to about 0.5% the volume of the aqueous dispersion. In yet another embodiment, the aqueous liquid dispersion can comprise a sweetening agent or flavoring agent in a concentration ranging from about 0.01% to about 1.0% the volume of the aqueous dispersion.

In addition to the additives listed above, the liquid formulations can also include inert diluents commonly used in the art, such as water or other solvents, solubilizing agents, and emulsifiers. Exemplary emulsifiers are ethyl alcohol, isopropyl alcohol, ethyl carbonate, ethyl acetate, benzyl alcohol, benzyl benzoate, propyleneglycol, 1,3-butyleneglycol, dimethylformamide, sodium lauryl sulfate, sodium doccusate, cholesterol, cholesterol esters, taurocholic acid, phosphotidylcholine, oils, such as cottonseed oil, groundnut oil, corn germ oil, olive oil, castor oil, and sesame oil, glycerol, tetrahydrofurfuryl alcohol, polyethylene glycols, fatty acid esters of sorbitan, or mixtures of these substances, and the like.

In some embodiments, the pharmaceutical formulations described herein can be self-emulsifying drug delivery systems (SEDDS). Emulsions are dispersions of one immiscible phase in another, usually in the form of droplets. Generally, emulsions are created by vigorous mechanical dispersion. SEDDS, as opposed to emulsions or microemulsions, spontaneously form emulsions when added to an excess of water without any external mechanical dispersion or agitation. An advantage of SEDDS is that only gentle mixing is required to distribute the droplets throughout the solution. Additionally, water or the aqueous phase can be added just prior to administration, which ensures stability of an unstable or hydrophobic active ingredient. Thus, the SEDDS provides an effective delivery system for oral and parenteral delivery of hydrophobic active ingredients. In some embodiments, SEDDS provide improvements in the bioavailability of hydrophobic active ingredients. Methods of producing self-emulsifying dosage forms are known in the art and include, but are not limited to, for example, U.S. Pat. Nos. 5,858,401, 6,667,048, and 6,960,563, each of which is specifically incorporated by reference.

It is to be appreciated that there is overlap between the above-listed additives used in the aqueous dispersions or suspensions described herein, since a given additive is often classified differently by different practitioners in the field, or is commonly used for any of several different functions. Thus, the above-listed additives should be taken as merely exemplary, and not limiting, of the types of additives that can be included in formulations described herein. The amounts of such additives can be readily determined by one skilled in the art, according to the particular properties desired.

### Injectable Formulations

In some embodiments, formulations suitable for intramuscular, subcutaneous, or intravenous injection include physiologically acceptable sterile aqueous or non-aqueous solutions, dispersions, suspensions or emulsions, and sterile powders for reconstitution into sterile injectable solutions or dispersions. Examples of suitable aqueous and non-aqueous carriers, diluents, solvents, or vehicles including water, ethanol, polyols (propyleneglycol, polyethylene-glycol, glycerol, cremophor and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions, and by the use of surfactants. In some embodiments, formulations suitable for subcutaneous injection also contain additives such as preserving, wetting, emulsifying, and dispensing agents. Prevention of the growth of microorganisms can be ensured by various antibacterial and antifungal agents, such as parabens, chlorobutanol, phenol, sorbic acid, and the like. In some embodiments, it is also desirable to include isotonic agents, such as sugars, sodium chloride, and the like. Prolonged absorption of the injectable pharmaceutical form can be brought about by the use of agents delaying absorption, such as aluminum monostearate and gelatin.

In some embodiments, for intravenous injections, compounds described herein are formulated in aqueous solutions, preferably in physiologically compatible buffers such as Hank's solution, Ringer's solution, or physiological saline buffer. For transmucosal administration, penetrants appropriate to the barrier to be permeated are used in the formulation. Such penetrants are generally known in the art. In some embodiments, for other parenteral injections, appropriate formulations include aqueous or nonaqueous solutions, preferably with physiologically compatible buffers or excipients. Such excipients are generally known in the art.

In some embodiments, parenteral injections involve bolus injection or continuous infusion. In some embodiments, formulations for injection are presented in unit dosage form, e.g., in ampoules or in multi-dose containers, with an added preservative. In some embodiments, the pharmaceutical composition described herein is in a form suitable for parenteral injection as a sterile suspensions, solutions or emulsions in oily or aqueous vehicles, and contains formulatory agents such as suspending, stabilizing and/or dispersing agents. Pharmaceutical formulations for parenteral administration include aqueous solutions of the active compounds in water-soluble form. Additionally, in some embodiments, suspensions of the active compounds are prepared as appropriate oily injection suspensions. Suitable lipophilic solvents or vehicles include fatty oils such as sesame oil, or synthetic fatty acid esters, such as ethyl oleate or triglycerides, or liposomes. In some embodiments, aqueous injection suspensions contain substances which increase the viscosity of the suspension, such as sodium carboxymethyl cellulose, sorbitol, or dextran. Optionally, in some embodiments, the suspension also contains suitable stabilizers or agents which increase the solubility of the compounds to allow for the preparation of highly concentrated solutions. Alternatively, in some embodiments, the active ingredient is in powder form for constitution with a suitable vehicle, e.g., sterile pyrogen-free water, before use.

### Intranasal Formulations

Intranasal formulations are known in the art and are described in, for example, U.S. Pat. Nos. 4,476,116, 5,116,817 and 6,391,452, each of which is specifically incorporated by reference. Formulations that include a Btk inhibitor (e.g. ibrutinib), which are prepared according to these and other techniques well-known in the art are prepared as solutions in saline, employing benzyl alcohol or other suitable preservatives, fluorocarbons, and/or other solubilizing or dispersing agents known in the art. See, for example, Ansel, H. C. et al., Pharmaceutical Dosage Forms and Drug Delivery Systems, Sixth Ed. (1995). Preferably these compositions and formulations are prepared with suitable nontoxic pharmaceutically acceptable ingredients. These ingredients are known to those skilled in the preparation of nasal dosage forms and some of these can be found in Remington: The Science and Practice of Pharmacy, 21st edition, 2005, a standard reference in the field. The choice of suitable carriers is highly dependent upon the exact nature of the nasal dosage form desired, e.g., solutions, suspensions, ointments, or gels. Nasal dosage forms generally contain large amounts of water in addition to the active ingredient. In some embodiments, minor amounts of other ingredients such as pH adjusters, emulsifiers or dispersing agents, preservatives, surfactants, gelling agents, or buffering and other stabilizing and solubilizing agents are also present. The nasal dosage form should be isotonic with nasal secretions.

In some embodiments, for administration by inhalation described herein, the pharmaceutical compositions are in a form as an aerosol, a mist or a powder. Pharmaceutical compositions described herein are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In some embodiments, in the case of a pressurized aerosol, the dosage unit is determined by providing a valve to deliver a metered amount. In some embodiments, capsules and cartridges of, such as, by way of example only, gelatin for use in an inhaler or insufflator are formulated containing a powder mix of the compound described herein and a suitable powder base such as lactose or starch.

### Buccal Formulations

In some embodiments, buccal formulations are administered using a variety of formulations known in the art. For example, such formulations include, but are not limited to, U.S. Pat. Nos. 4,229,447, 4,596,795, 4,755,386, and 5,739,136, each of which is specifically incorporated by reference. In addition, the buccal dosage forms described herein can further include a bioerodible (hydrolysable) polymeric carrier that also serves to adhere the dosage form to the buccal mucosa. The buccal dosage form is fabricated so as to erode gradually over a predetermined time period, wherein the delivery is provided essentially throughout. Buccal drug delivery, as will be appreciated by those skilled in the art, avoids the disadvantages encountered with oral drug administration, e.g., slow absorption, degradation of the active agent by fluids present in the gastrointestinal tract and/or first-pass inactivation in the liver. With regard to the bioerodible (hydrolysable) polymeric carrier, it will be appreciated that virtually any such carrier can be used, so long as the desired drug release profile is not compromised, and the carrier is compatible with ibrutinib, and any other components that are present in the buccal dosage unit. Generally, the polymeric carrier comprises hydrophilic (water-soluble and water-swellable) polymers that adhere to the wet surface of the buccal mucosa. Examples of polymeric carriers useful herein include acrylic acid polymers and co, e.g., those known as “carbomers” (Carbopol®, which can be obtained from B.F. Goodrich, is one such polymer). In some embodiments, other components are also incorporated into the buccal dosage forms described herein include, but are not limited to, disintegrants, diluents, binders, lubricants, flavoring, colorants, preservatives, and the like. In some embodiments, for buccal or sublingual administration, the compositions are in the form of tablets, lozenges, or gels formulated in a conventional manner.

### Transdermal Formulations

In some embodiments, transdermal formulations described herein are administered using a variety of devices which have been described in the art. For example, such devices include, but are not limited to, U.S. Pat. Nos. 3,598,122, 3,598,123, 3,710,795, 3,731,683, 3,742,951, 3,814,097, 3,921,636, 3,972,995, 3,993,072, 3,993,073, 3,996,934, 4,031,894, 4,060,084, 4,069,307, 4,077,407, 4,201,211, 4,230,105, 4,292,299, 4,292,303, 5,336,168, 5,665,378, 5,837,280, 5,869,090, 6,923,983, 6,929,801 and 6,946,144, each of which is specifically incorporated by reference in its entirety.

In some embodiments, the transdermal dosage forms described herein incorporate certain pharmaceutically acceptable excipients which are conventional in the art. In some embodiments, the transdermal formulations described herein include at least three components: (1) a formulation of a compound of ibrutinib; (2) a penetration enhancer; and (3) an aqueous adjuvant. In addition, transdermal formulations can include additional components such as, but not limited to, gelling agents, creams and ointment bases, and the like. In some embodiments, the transdermal formulation can further include a woven or non-woven backing material to enhance absorption and prevent the removal of the transdermal formulation from the skin. In other embodiments, the transdermal formulations described herein can maintain a saturated or supersaturated state to promote diffusion into the skin.

In some embodiments, formulations suitable for transdermal administration of compounds described herein employ transdermal delivery devices and transdermal delivery patches and can be lipophilic emulsions or buffered, aqueous solutions, dissolved and/or dispersed in a polymer or an adhesive. In some embodiments, such patches are constructed for continuous, pulsatile, or on demand delivery of pharmaceutical agents. Still further, transdermal delivery of the compounds described herein can be accomplished by means of iontophoretic patches and the like. Additionally, transdermal patches can provide controlled delivery of ibrutinib. The rate of absorption can be slowed by using rate-controlling membranes or by trapping the compound within a polymer matrix or gel. Conversely, absorption enhancers can be used to increase absorption. An absorption enhancer or carrier can include absorbable pharmaceutically acceptable solvents to assist passage through the skin. For example, transdermal devices are in the form of a bandage comprising a backing member, a reservoir containing the compound optionally with carriers, optionally a rate controlling barrier to deliver the compound to the skin of the host at a controlled and predetermined rate over a prolonged period of time, and means to secure the device to the skin.

### Other Formulations

In certain embodiments, delivery systems for pharmaceutical compounds are employed, such as, for example, liposomes and emulsions. In certain embodiments, compositions provided herein can also include an mucoadhesive polymer, selected from among, for example, carboxymethylcellulose, carbomer (acrylic acid polymer), poly(methylmethacrylate), polyacrylamide, polycarbophil, acrylic acid/butyl acrylate copolymer, sodium alginate and dextran.

In some embodiments, the compounds described herein are administered topically and can be formulated into a variety of topically administrable compositions, such as solutions, suspensions, lotions, gels, pastes, medicated sticks, balms, creams or ointments. Such pharmaceutical compounds can contain solubilizers, stabilizers, tonicity enhancing agents, buffers and preservatives.

In some embodiments, the compounds described herein are formulated in rectal compositions such as enemas, rectal gels, rectal foams, rectal aerosols, suppositories, jelly suppositories, or retention enemas, containing conventional suppository bases such as cocoa butter or other glycerides, as well as synthetic polymers such as polyvinylpyrrolidone, PEG, and the like. In suppository forms of the compositions, a low-melting wax such as, but not limited to, a mixture of fatty acid glycerides, optionally in combination with cocoa butter is first melted.

### Kits/Articles of Manufacture

Described herein are kits for preventing or delaying the onset of Type 1 Diabetes, inhibiting the maturation of anti-insulin B cells, or decreasing the population of anti-insulin B cells, in an individual in need thereof comprising administering to the individual a composition comprising a therapeutically-effective amount of a covalent Btk inhibitor (e.g., an irreversible covalent Btk inhibitor, e.g., ibrutinib).

For use in the therapeutic applications described herein, kits and articles of manufacture are also described herein. In some embodiments, such kits include a carrier, package, or container that is compartmentalized to receive one or more containers such as vials, tubes, and the like, each of the container(s) including one of the separate elements to be used in a method described herein. Suitable containers include, for example, bottles, vials, syringes, and test tubes. The containers can be formed from a variety of materials such as glass or plastic.

The articles of manufacture provided herein contain packaging materials. Examples of pharmaceutical packaging materials include, but are not limited to, blister packs, bottles, tubes, inhalers, pumps, bags, vials, containers, syringes, bottles, and any packaging material suitable for a selected formulation and intended mode of administration and treatment. A wide array of formulations of the compounds and compositions provided herein are contemplated as are a variety of treatments for any disorder that benefit by inhibition of Btk, or in which Btk is a mediator or contributor to the symptoms or cause.

The container(s) optionally have a sterile access port (for example the container is an intravenous solution bag or a vial having a stopper pierceable by a hypodermic injection needle). Such kits optionally comprising a compound with an identifying description or label or instructions relating to its use in the methods described herein.

A kit will typically include one or more additional containers, each with one or more of various materials (such as reagents, optionally in concentrated form, and/or devices) desirable from a commercial and user standpoint for use of a compound described herein. Non-limiting examples of such materials include, but not limited to, buffers, diluents, filters, needles, syringes; carrier, package, container, vial and/or tube labels listing contents and/or instructions for use, and package inserts with instructions for use. A set of instructions will also typically be included.

In some embodiments, a label is on or associated with the container. A label can be on a container when letters, numbers or other characters forming the label are attached, molded or etched into the container itself; a label can be associated with a container when it is present within a receptacle or carrier that also holds the container, e.g., as a package insert. A label can be used to indicate that the contents are to be used for a specific therapeutic application. The label can also indicate directions for use of the contents, such as in the methods described herein.

In certain embodiments, a pharmaceutical composition comprising the covalent Btk inhibitor (e.g., an irreversible covalent Btk inhibitor, e.g., ibrutinib) is presented in a pack or dispenser device which can contain one or more unit dosage forms. The pack can for example contain metal or plastic foil, such as a blister pack. The pack or dispenser device can be accompanied by instructions for administration. The pack or dispenser can also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, can be the labeling approved by the U.S. Food and Drug Administration for prescription drugs, or the approved product insert. Compositions containing a compound provided herein formulated in a compatible pharmaceutical carrier can also be prepared, placed in an appropriate container, and labeled for treatment of an indicated condition.

### EXAMPLES

The following specific and non-limiting examples are to be construed as merely illustrative, and do not limit the present disclosure in any way whatsoever.

### Example 1

### Materials and Methods

Site-Directed Anti-Insulin BCR Targeting and Mouse Generation

Anti-insulin Vk125 was targeted to the Igklocus to generate Vk125SD^{Neo} mice as follows. The pSVG-125Vk vector used to generate Vk125Tg mice was digested with EcoRI and SacII to liberate the Vk125/Jk5 fragment which was subcloned into the pBluescript SK(−) vector (Stratagene) and was verified through sequencing. Vk125/Jk5 was then amplified from this

NotI FWD
5′ AAGCGGCCGCAGGGTCTGTCGAT

vector using the primers and SalI REV 5′AAAGTCGACGGATGAGTCTCCTTCTC. The 1.6 kbVk125/Jk5 fragment was subsequently cloned into NotI and SalI sites in the pVKR3-83-2^{Neo} targeting vector used to generate 3-83k mice that lack downstream Jk segments on the targeted allele and the pVKR-Vk125SD^{Neo} targeting construct was verified through sequencing.

The pVKR-V.125SD^{Neo} targeting vector was linearized through digestion with ClaI and electroporated into mouse TL1 embryonic stem (ES) cells (129 strain). ES cell clones were selected with 200 μg/mL G418 and 2 μM Gancyclovir. Double-resistant colony DNA was SacI digested and the endogenous allele (5.5 kb) and the targeted allele (6.3 kb) were identified in Southern blot using the 1.6 kb EcoRI probe. Positive clones (33 of 458 ESC clones screened) were subsequently verified through PCR using FWD 5′ TCTGCAAATGTCTGATGAGT and REV 5′ CTCGTGCTTTACGGTATCGC primers. Two clones were injected into blastocysts that were transplanted into pseudopregnant females. ESC electroporation, selection, expansion, DNA isolation and digestion for initial Southern blot screening, and injection of ES cells into blastocysts were performed by the Vanderbilt Transgenic Mouse/Embryonic Stem Cell Shared Resource.

Chimeric male offspring were intercrossed with C57BL/6 females and progeny were screened through PCR and Southern blot analysis as above to confirm the presence of the targeted allele. PCR was also used to confirm that the intact Vk125/Jk5 was present in targeted mice (FWD 5′ AATGGATTTTCAGGTGCAGAT and REV 5′ GCTCCAGCTTGGTCCCAGCA). Six chimeric founder males were backcrossed to WT C57BL/6 females to generate mice carrying the targeted Vk125 allele. The six independent lines of progeny were all found to contain the targeted allele, based on PCR and Southern blot analysis. Vk125SD^{Neo} mice were intercrossed with VH125Tg C57BL/6 females [Cg-Tg(Igh-6/Igh-V125)2Jwt/JwtJ] to generate VH125/Vk125SD^{Neo} offspring. Progeny from all six founder lines exhibited similar percentages of “edited” non-insulin-binding B cells as assessed by flow cytometry, which remained consistent at various stages of backcrossing. One line was subsequently used for continued backcrossing To C57BL/6 and NOD and in experiments shown.

Animals and Disease Studies.

125Tg/NOD mice were crossed with Btk-deficient NOD mice to produce 125Tg/NOD Btk−/− mice that were backcrossed onto the NOD strain ≧10 generations. Female mice were monitored for blood glucose levels weekly and considered diabetic at the first of two consecutive readings above 200 mg/dL. Vk125SD^{Neo} mice were intercrossed with VH125Tg mice and Btk−/− mice to generate V_{H}125Tg/Vk125SD^{Neo} Btk-sufficient and V_{H}125Tg/Vk125SD^{Neo} Btk-deficient mice that were backcrossed onto the NOD strain ≧7 generations. All mice were housed under specific pathogen-free conditions.

BTK Inhibitor Treatment.

125Tg/NOD mice were fed Ibrutinib (PCI-32765, Pharmacyclics, 0.24 g Ibrutinib/kg chow) or placebo chow ad libitim for 5-10 weeks. An average of 38 mg/kg Ibrutinib inhibitor was consumed per day, based on food weight consumed throughout the duration of the study and the number of mice per cage. This is above the dosage that ensures 99% inhibitor activity. B cell subsets were assessed by flow cytometry in freshly isolated organs as outlined below.

Cell Isolation, Flow Cytometry, and Antibodies.

BM was eluted from long bones, and spleens and pancreatic draining lymph nodes (PLN) were macerated with HBSS (Invitrogen) plus 10% FBS (HyClone). Red blood cells were lysed using Tris-NH₄Cl. Freshly isolated pancreata were digested with 3 mL of 1 mg/mL collagenase P diluted in HBSS for 30 min at 37° C., then tissue was disrupted using an 18-gauge needle. Ice cold HBSS plus 10% FBS was immediately added to inhibit collagenase activity. Cells were directly analyzed by flow cytometry. Flow cytometry Ab reagents were reactive with B220 (6B2), IgM^{a} (DS-1), CD19 (1D3), CD21 (7G6), CD23 (B3B4), CD93 (AA4.1), or IgM chain-specific) (Life Technologies). Biotin N-hydroxysuccinimide ester (Sigma) was used to biotinylate human insulin (Sigma) at pH 8.0 in bicine buffer. Streptavidin reagents (BD Biosciences) were used to detect biotinylated reagents. 7AAD (BD Biosciences) was used to identify dead cells. Sample acquisition was performed using an LSR II flow cytometer (BD Biosciences) and FlowJo software (Treestar) was used for analysis.

Ca²⁺ Mobilization Assay.

Bone marrow cells were harvested and grown in bone marrow culture media with 15 ng/mL recombinant human IL-7 (PeproTech) for 5 d, then with no IL-7 for an additional 2 d to promote differentiation. Intracellular Ca²⁺ mobilization was measured by determining changes in the ratio of bound/free fura 2-AM fluorescence intensities using a FlexStation II fluorimeter (Molecular Devices). Basal readings were taken for 45 s prior to stimulation. Cells were stimulated with 1 μg/mL anti-IgM [F(ab′)₂ goat anti-mouse μ-chain, (Jackson ImmunoResearch Labs, Inc.)] and well fluorescence was monitored at 37° C.

In Vivo Labeling of Sinusoidal Bone Marrow B Cells.

Lateral tail veins of mice were injected with 1 μg CD19-PE (BD Pharmingen) in 200 sterile 1×PBS. Mice were sacrificed after 2 min and bone marrow was immediately eluted from femurs. Cells were isolated as described above and were subsequently incubated with indicated Abs to stain cell surface markers, together with CD19-APC to help delineate all CD19⁺ cells, including sinusoidal B cells preferentially labeled with CD19-PE.

BCR Internalization Assay.

Antigen-internalization was performed. Freshly isolated bone marrow and spleen cells were incubated with biotinylated insulin 30 min on ice to occupy BCR. After washing away excess biotinylated insulin, cells were incubated in complete media at 37° C. for 0-10 min, at which point cells were stained with streptavidin-fluorochrome, as well as other indicated Abs. The relative surface level of biotinylated insulin or IgM was determined by dividing the MFI at each time point by the MFI at t=0, such that 100% represents no change in surface expression.

Results

Anergic, Autoreactive B Cells Depend Upon BTK.

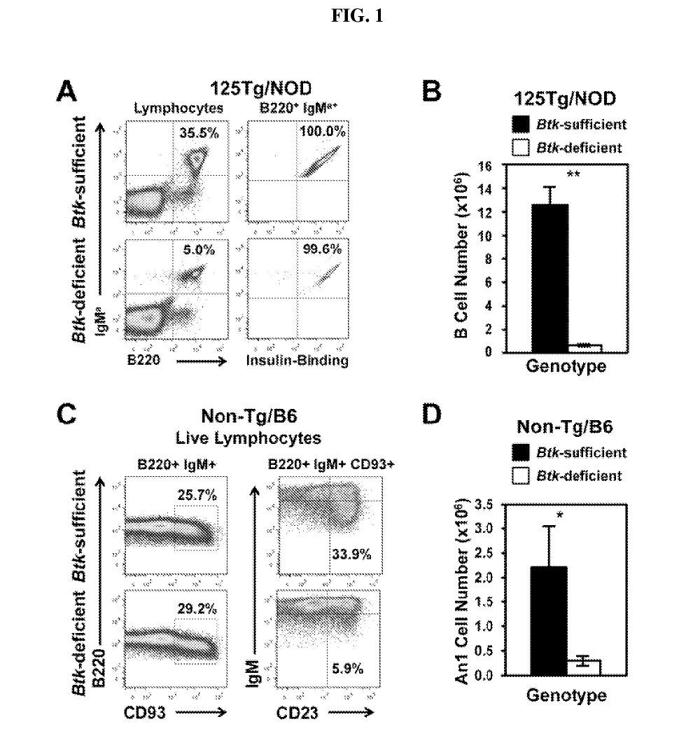

Btk-deficiency was crossed onto 125Tg mice, on both C57BL/6 and NOD backgrounds. FIG. 1A shows representative flow cytometry dotplots from Btk-deficient 125Tg/NOD mice vs. their Btk-sufficient counterparts. Btk-deficient 125Tg mice were found to have severely reduced spleen B cell compartments (0.63±0.10×10⁶ vs. 12.6±1.5×10⁶, p<0.001), retaining only 5% normal numbers of insulin-binding B cells (FIG. 1B and Table II). Results in C57BL/6 mice do not differ from those of NOD (not shown). To extend these findings to anergic B cells in a fully polyclonal repertoire, we also examined the effect of Btk-deficiency on the anergic, autoreactive-prone An1 subset in non-transgenic mice. The An1 subset is CD93⁺/CD23+/IgM^{lo}. This subset cannot be examined in NOD mice, due to technical issues with the AA4.1 (anti-CD93) antibody, so studies were performed using C57BL/6 mice. FIG. 1C shows representative dot-plots of B220⁺ IgM⁺ live lymphocytes (left) gated on CD93⁺ cells depicting the An1 subset (CD23⁺/IgM^{lo}), right, from wild type (WT) and Btk-deficient mice. FIG. 1D and Table III shows that Btk-deficient mice have significantly reduced percentages and numbers of An1 B cells (p<0.001). These data are similar to previously published findings in the Btk-deficient BALB.xid model, in which this subset, then defined as T3, was also found to be decreased. Thus, Btk-deficiency dramatically decreases the numbers of autoreactive-prone, anergic B cells, in both a naturally-occurring population, and in a well-studied anergic, anti-insulin transgenic model.

TABLE III
An1 B cell subset numbers (×10⁴ cells) and percentages.

| | | | t test p |
|---|---|---|---|
| Organ | Btk-Sufficient | Btk-Deficient | value |
| Anl Subset Percentage^{a,b} | 7.0 ± 1.2 | 1.7 ± 0.1 | <0.001 |
| Anl Subset Number^{b} | 2.2 ± 0.8 | 0.3 ± 0.1 | <0.001 |

^{a}Percentage of total splenocytes
^{b}An1 subset identified as B220⁺ CD93 (AA4.1)⁺ CD23⁺ IgM^{low}

BTK is Dispensable for Development of Immature Anti-Insulin B Cells.

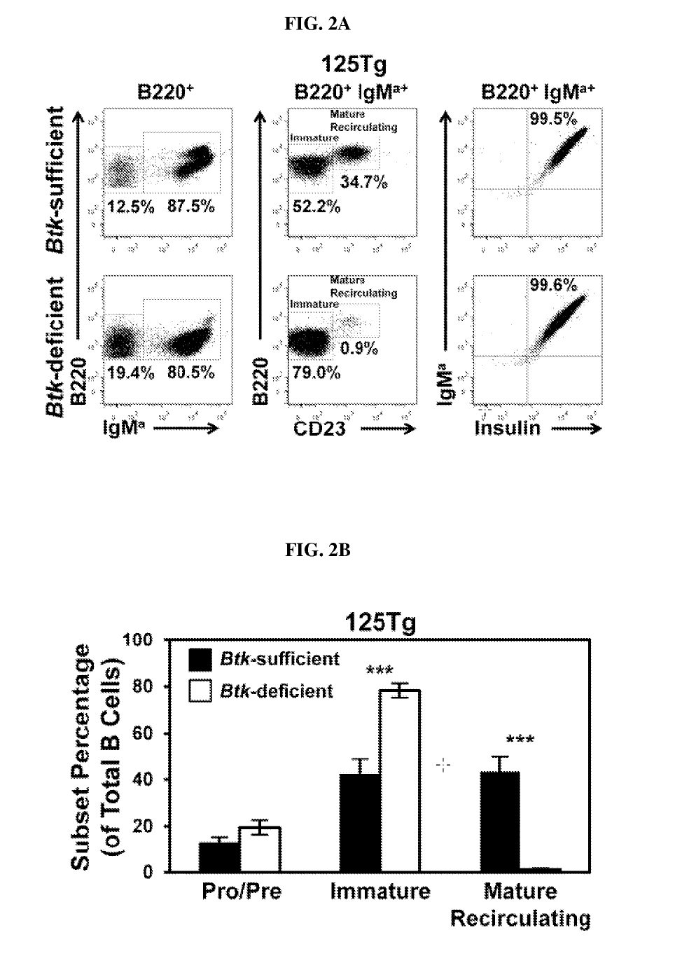

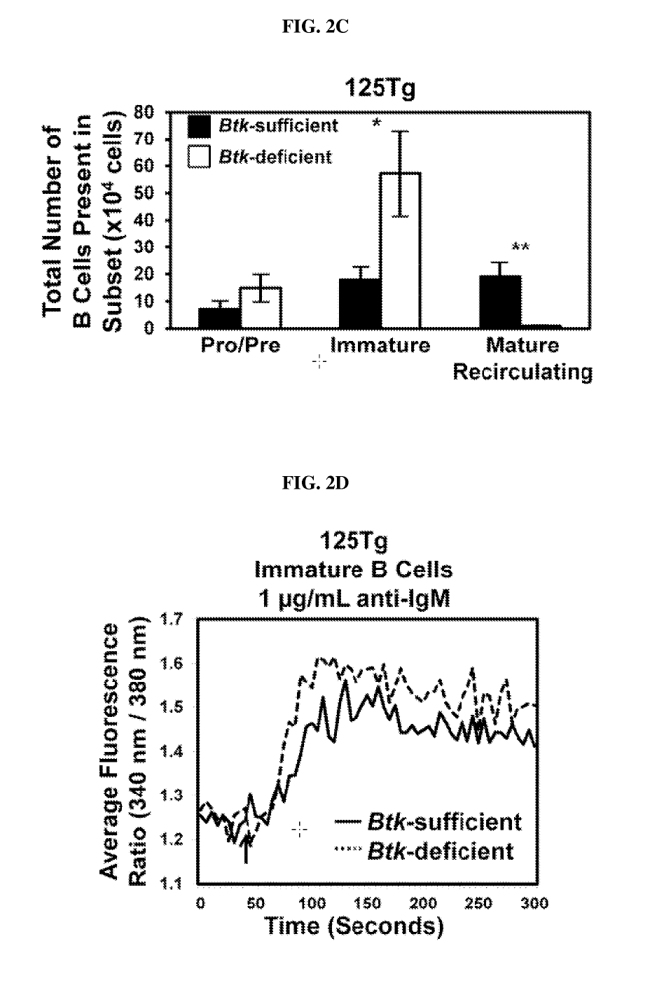

B cell developmental subsets were identified in freshly isolated bone marrow of Btk-sufficient or deficient 125Tg/NOD mice using flow cytometry to detect B220, IgM¹, and CD23 expression. Insulin binding specificity was confirmed with biotinylated insulin staining detected by a streptavidin-fluorochrome conjugate. Representative plots are shown in FIG. 2A, and the average frequency (FIG. 2B and Table I) or total number (FIG. 2C and Table II) ±SEM of pro and pre (B220⁺ IgM^{a-}), immature (B220^{mid} IgM^{a+} CD23⁻), or mature recirculating (B220^{high} IgM^{a+} CD23⁺) B cells is shown. Btk-deficiency confers comparable or elevated frequency and number of immature B cells in the bone marrow of 125Tg/NOD mice. In contrast, mature recirculating B cell numbers are significantly reduced (19.0±5.1×10⁴ vs. 0.9±0.2×10⁴, p=0.008).

TABLE I
125Tg B cell subset percentages

| | | Btk- | Btk- | t test p |
|---|---|---|---|---|
| Organ | B Cell Subset | Sufficient | Deficient | value |
| Bone Marrow | Total | 2.0 ± 0.4 | 2.7 ± 0.5 | 0.35 |
| Bone Marrow | Pro/Pre | 13.2 ± 1.9 | 19.5 ± 2.9 | 0.08 |
| Bone Marrow | Immature | 42.4 ± 6.0 | 78.3 ± 2.8 | <0.001 |
| Bone Marrow | Mature | 43.4 ± 6.1 | 1.2 ± 0.2 | <0.001 |
| | Recirculating | | | |
| Spleen | Total | 14.0 ± 1.5 | 1.1 ± 0.1 | <0.001 |
| Spleen | T1 | 2.6 ± 0.5 | 16.5 ± 2.7 | <0.001 |
| Spleen | T2 | 10.0 ± 1.8 | 22.2 ± 2.8 | 0.002 |
| Spleen | Follicular | 25.1 ± 3.2 | 3.9 ± 0.3 | <0.001 |
| Spleen | Pre-Marginal Zone | 4.9 ± 0.9 | 2.5 ± 0.5 | 0.02 |
| Spleen | Marginal Zone | 57.5 ± 3.2 | 55.0 ± 4.2 | 0.65 |
| PLN | Total | 8.2 ± 4.0 | 0.4 ± 0.3 | <0.001 |
| Pancreas | Total | 0.3 ± 0.1 | 0.01 ± 0.00 | 0.03 |

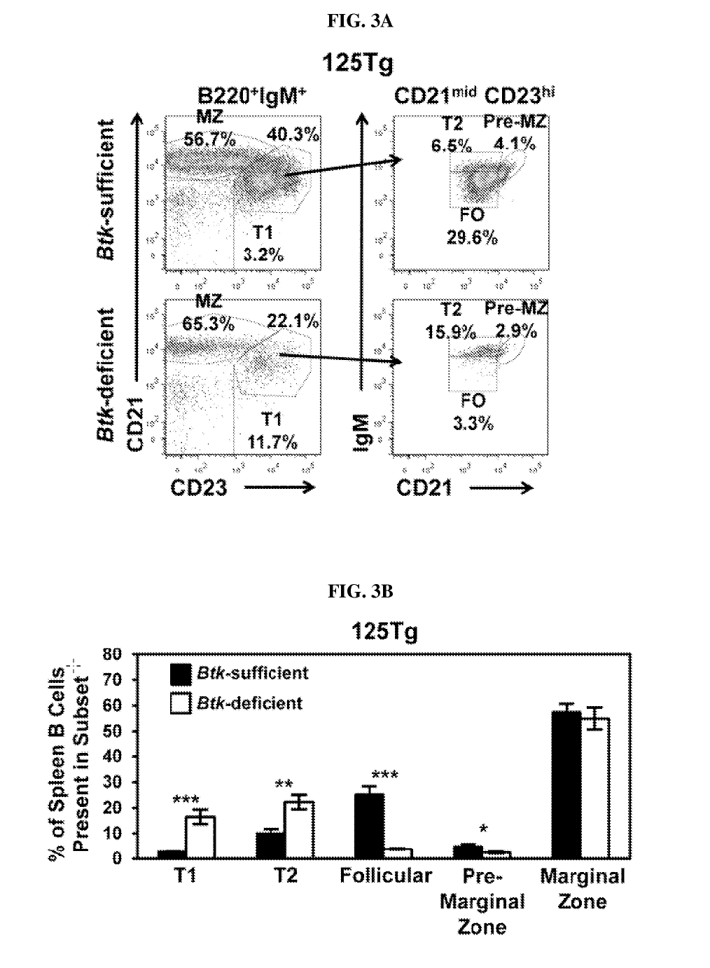

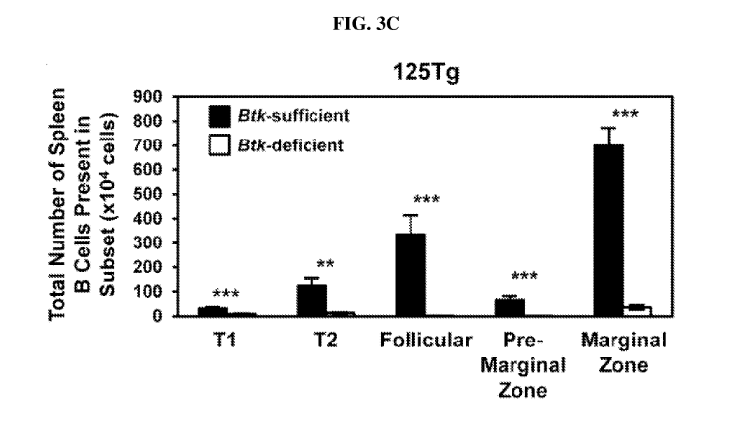

^{a}“Total” indicates B lymphocyte percentage of total cells in the indicated organ
^{b}B cell subsets identified as in FIG. 2-3

TABLE II
125Tg B cell subset numbers (×10⁴ cells)

| | | Btk- | Btk- | t test p |
|---|---|---|---|---|
| Organ | B Cell Subset | Sufficient | Deficient | value |
| Bone Marrow | Total | 44.4 ± 10.6 | 73.8 ± 20.5 | 0.19 |
| Bone Marrow | Pro/Pre | 7.1 ± 2.8 | 14.9 ± 4.9 | 0.16 |
| Bone Marrow | Immature | 17.9 ± 4.6 | 57.3 ± 15.6 | 0.013 |
| Bone Marrow | Mature | 19.0 ± 5.1 | 0.9 ± 0.2 | 0.008 |
| | Recirculating | | | |
| Spleen | Total | 1256.7 ± 152.5 | 62.8 ± 10.1 | <0.001 |
| Spleen | T1 | 31.9 ± 5.4 | 9.2 + 1.4 | <0.001 |
| Spleen | T2 | 124.1 ± 30.3 | 13.0 + 1.8 | 0.0011 |
| Spleen | Follicular | 335.1 ± 79.1 | 2.2 ± 0.3 | <0.001 |
| Spleen | Pre-Marginal | 66.0 ± 16.0 | 1.7 ± 0.4 | <0.001 |
| | Zone | | | |
| Spleen | Marginal Zone | 699.6 ± 72.4 | 36.7 ± 8.2 | <0.001 |
| PLN | Total | 33.9 ± 19.7 | 1.2 ± 1.1 | <0.001 |
| Pancreas | Total | 4.1 ± 1.3 | 0.3 ± 0.1 | 0.014 |

^{a}B cell subsets identified as in FIG. 2-3

BCR-Mediated Calcium Flux in Immature Anti-Insulin B Cells does not Require BTK.

BCR signaling is known to be impaired in mature Btk-deficient B cells. However, the fact that immature B cell development is unimpeded in Btk-deficient 125Tg/NOD mice raises the question of whether BCR signaling in anti-insulin B cells may occur independently of BTK at this developmental stage. To test this, naïve immature 125Tg B cells were generated using IL-7-driven culture. BCR-induced calcium mobilization was then measured in Btk-sufficient and deficient immature cells following stimulation with anti-IgM. Interestingly, comparable calcium mobilization was observed in 125Tg/NOD immature B cells regardless of BTK status (FIG. 2D). Impaired calcium flux was observed in mature Btk-deficient 125Tg B cells, as expected (not shown). Consistent with the above data on B cell development, these data show that Btk-deficiency does not impair calcium mobilization following BCR stimulation in immature 125Tg B cells, highlighting a major difference in signaling between immature and mature anti-insulin B cells.

Btk-Deficiency Results in Loss of Anti-Insulin B Cells at Every Developmental Stage in the Spleen.

Btk-deficiency in wild type NOD mice confers a 5% reduction in splenic B cell numbers. In 125Tg/NOD mice, however, Btk-deficiency results in >90% loss of B cells, as shown in FIG. 1A. In NOD mice with endogenous BCRs, Btk-deficiency causes a partial block at the T2 to follicular B cell transition, as well as a small reduction in marginal zone B cell numbers. To address whether Btk-deficiency affects anti-insulin B cell development differently, spleen B cell subsets were compared in Btk-sufficient and deficient 125Tg/NOD mice. FIG. 3A shows the flow cytometry gating scheme to detect T1 (CD21^{low} CD23^{low}), T2 (CD21^{mid} CD23^{high} IgM^{high}), follicular (CD21^{mid} CD23^{high} IgM^{mid}), pre-marginal zone (CD21^{high} CD23^{high} IgM^{high}), and marginal zone (CD21^{high} CD23^{mid}) B cells. Quantification of subset proportions for each genotype (FIG. 3B and Table I) shows that both early and late transitional components are relatively overrepresented in the setting of Btk-deficiency, suggesting that there is a block in maturation through both of these checkpoints.

Quantification of total cell numbers, however, shows that anti-insulin B cells are lost at all phases of development (FIG. 3C, and Table II). The number of follicular B cells in 125Tg/NOD Btk-deficient mice was reduced 99% compared to 125Tg/NOD Btk-sufficient mice (2.2±0.3×10⁴ vs. 335.1±79.1×10⁴, p<0.001). While the frequency of marginal zone B cells was not different, the total number of marginal zone B cells was reduced by 95% in Btk-deficient 125Tg/NOD mice (36.7±8.2×10⁴ vs. 699.6±72.4×10⁴, p<0.001). The cell populations from which these mature subsets arise are also markedly reduced: the early transitional T1 subset by 71% (9.2±1.4×10⁴ vs. 31.9±5.4×10⁴, p<0.001), late transitional T2 subset by 90% (13.0±1.8×10⁴ vs. 124.1±30.3×10⁴, p=0.0011), and pre-marginal zone by 89% (1.7±0.4×10⁴ vs. 66.0±16.0×10⁴, p<0.001). These data show that anti-insulin B cell maturation and/or survival is more profoundly impaired by loss of BTK than is observed in the polyclonal repertoire of WT NOD mice.

Kinase Inhibition of BTK Reduces Mature, but not Transitional, Anti-Insulin B Cells.

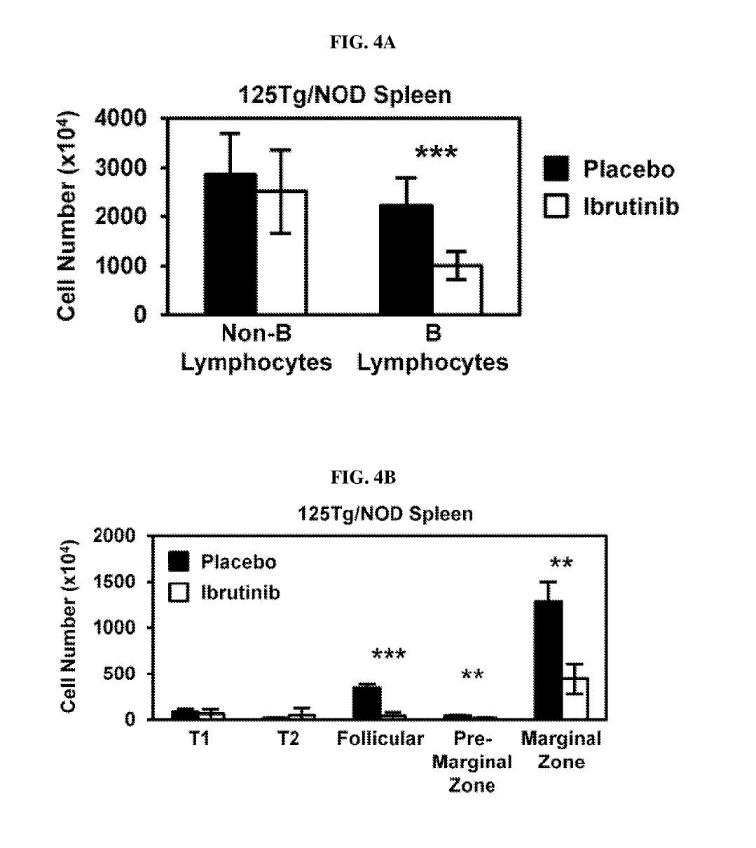

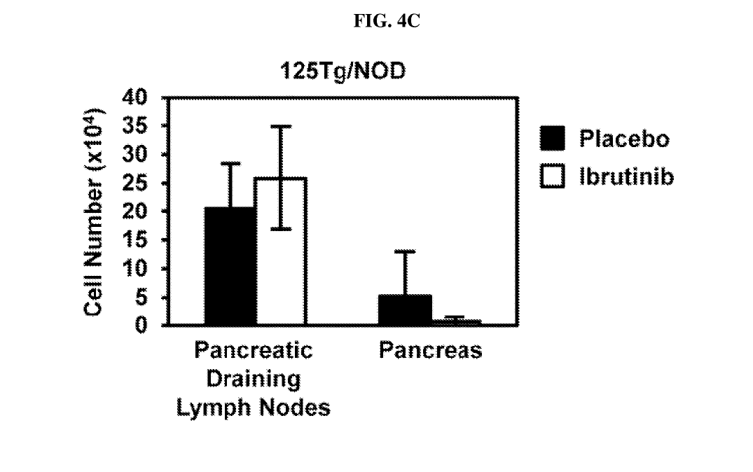

BTK is a complex protein with adapter as well as kinase functions that have independent effects on various aspects of B cell development and function. Therefore, a kinase inhibitor, Ibrutinib (PCI-32765) was used to test the effects of kinase inhibition on anti-insulin B cell development and survival. Ibrutinib chow or placebo chow was fed to 125Tg/NOD mice for 5-10 weeks. The average inhibitor dosage was 38 mg/kg (sufficient to elicit 99% inhibitor function). Flow cytometry analysis of freshly isolated splenocytes was used to identify B cell subsets as in FIG. 3. These data mirror the developmental alteration seen in 125Tg/NOD Btk-deficient mice (FIG. 4). The number of B lymphocytes was decreased in ibrutinib vs. placebo chow fed mice (10.1±2.8×10⁶ vs. 22.2±5.8×10⁶, p<0.001), whereas no difference was observed in non-B lymphocyte numbers (25.1±8.5×10⁶ vs. 28.6±8.3×10⁶, FIG. 4A). As shown in FIG. 4B, no difference was observed in T1 (63±47×104 vs. 83±32×10⁴, p=0.57) or T2 (49±76×10⁴ vs. 17±3×10⁴, p=0.51) B cell subset numbers in the spleen. However, an 88% reduction in follicular (41±37×10⁴ vs. 345±38×10⁴, p<0.001), 60% reduction in pre-marginal zone (17±5×10⁴ vs. 43±6×10⁴, p=0.0015), and 65% reduction in marginal zone (443±164×10⁴ vs. 1283±217×10⁴, p=0.002) B cell numbers was observed. In contrast, no significant difference was observed in the number of B cells in the pancreatic draining lymph nodes (26±9×10⁴ vs. 21±8×10⁴, p=0.49) or pancreas (0.66±0.80×10⁴ vs. 5.2±7.7×10⁴, p=0.36, FIG. 4C) of 125Tg/NOD mice treated with Ibrutinib. These data show that inhibition of BTK kinase function impairs the maturation and/or survival of anti-insulin B lymphocytes, but that Ibrutinib does not block their trafficking to pancreatic draining lymph nodes or pancreas

Anti-Insulin B Cells are Preferentially Susceptible to Btk-Deficiency.

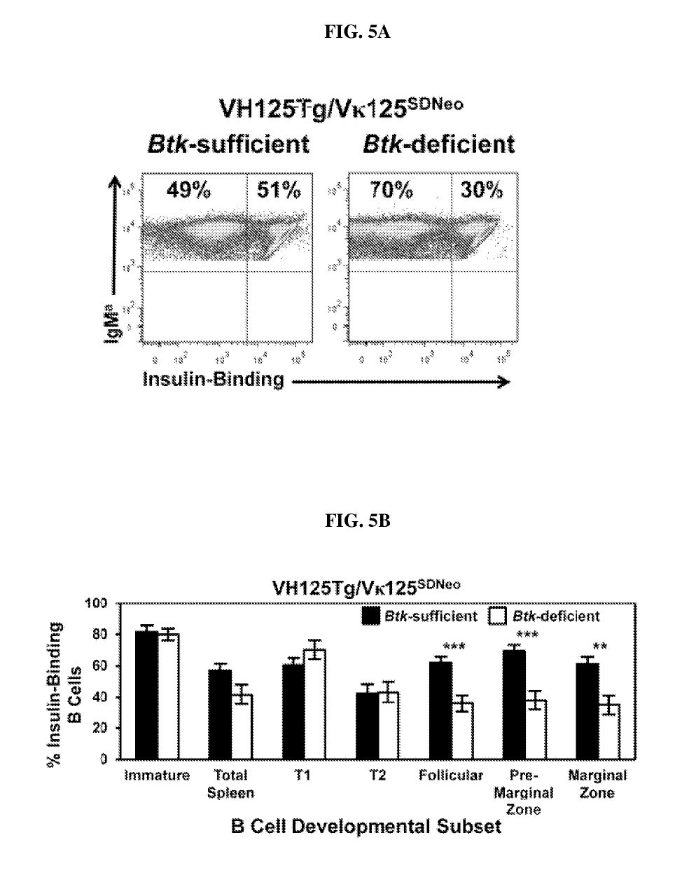

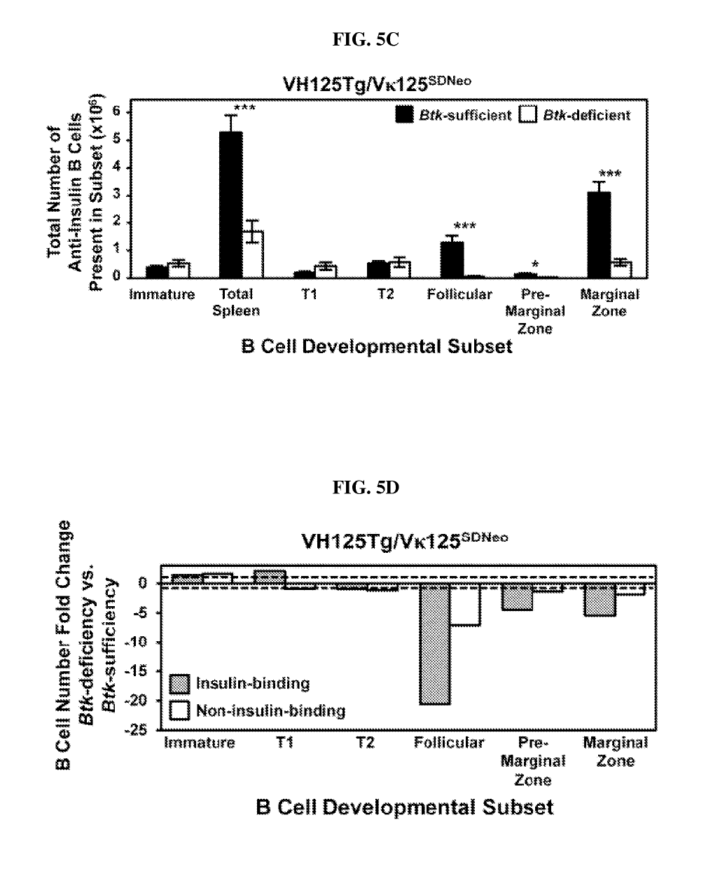

The dramatic reduction in mature B cell subsets in 125Tg/NOD compared to WT/NOD Btk-deficient mice suggests that autoreactive (anti-insulin) B cells rely more heavily on BTK-mediated signaling than do non-autoreactive B cells. However, the presence of a BCR transgene may have unrecognized effects that are unrelated to antigen-specificity. Therefore, we used a novel BCR transgenic model, in which an anti-insulin light chain is targeted to the Igκ locus to allow receptor editing. This model, VH125Tg/Vκ125^{SDNeo}, provides the unique opportunity to track a substantial population of anti-insulin B cells (˜50%) that develop alongside an equally large competing repertoire harboring the same BCR transgene (FIG. 5A, freshly isolated spleen is shown). Developmental subsets were characterized in the bone marrow and spleens of V_{H}125Tg/Vκ125^{SDNeo}/NOD Btk-deficient or sufficient mice using flow cytometry (FIG. 5 and Tables IV-VI). Btk-deficiency has no effect on the proportion or number of insulin-binding B cells found among immature cells in the bone marrow (FIG. 5B-C, Tables IV and VI). Interestingly, the proportion and number of insulin-binding B cells in the transitional T1 and T2 stages in the spleen is unchanged by BTK loss in this model. However, the proportions and numbers of insulin-binding Btk-deficient B cells drop markedly with BTK loss in the follicular (42%, 95%, respectively), pre-marginal zone (45%, 78%), and marginal zone subsets (43%, 82%) (FIG. 5B-C, Tables IV and VI).

TABLE IV
VH125Tg/V_{K}125^{SDNeo} B cell subset percentage

| | | | | | Brk- | Brk- | |
|---|---|---|---|---|---|---|---|
| | | Btk- | Btk- | | Sufficient | Deficient | |
| | B Celt | Sufficient | Deficient | t test | Insulin- | Insulin- | t test p |
| Organ | Subset | Total | Total | p value | Binding | Binding | value |
| Bone | Total | 4.7 ± 0.2 | 5.5 ± 0.6 | 0.26 | | | |
| Marrow | | | | | | | |
| Bone | Pro/Pre | 29.9 ± 3.3 | 38.9 ± 4.8 | 0.14 | | | |
| Marrow | | | | | | | |
| Bone | Immature | 46.7 ± 4.1 | 49.0 ± 4.1 | 0.70 | 81.6 ± 3.9 | 79.8 ± 3.6 | 0.75 |
| Marrow | | | | | | | |
| Bone | Mature | 15.6 ± 3.3 | 6.2 ± 1.2 | 0.02 | 63.3 ± 5.7 | 44.8 ± 5.4 | 0.03 |
| Marrow | Recirculating | | | | | | |
| Spleen | Total | 13.2 ± 1.0 | 6.4 ± 0.7 | <0.001 | 56.9 ± 4.4 | 41.5 ± 6.3 | 0.057 |
| Spleen | T1 | 4.1 ± 0.8 | 15.6 ± 2.7 | <0.001 | 60.5 ± 4.2 | 70.2 ± 5.9 | 0.19 |
| Spleen | T2 | 14.8 ± 0.8 | 30.1 ± 3.2 | <0.001 | 42.3 ± 5.9 | 43.1 ± 6.7 | 0.93 |
| Spleen | Follicular | 21.9 ± 3.0 | 4.4 ± 0.6 | >0.001 | 61.9 ± 3.6 | 35.7 ± 5.3 | >0.001 |
| Spleen | Pre-Marginal | 2.5 ± 0.7 | 2.8 ± 0.5 | 0.72 | 69.4 ± 3.9 | 38.0 ± 6.1 | >0.001 |
| | Zone | | | | | | |
| Spleen | Marginal | 54.2 ± 3.0 | 42.3 ± 3.8 | 0.02 | 61.2 ± 4.6 | 34.7 ± 6.3 | 0.003 |
| | Zone | | | | | | |
| Pancreatic | Total | 5.2 ± 0.7 | 1.9 ± 0.5 | <0.001 | 43.9 ± 1.6 | 44.1 ± 7.2 | 0.98 |
| Draining | | | | | | | |
| Lymph | | | | | | | |
| Nodes | | | | | | | |
| Pancreas | Total | 0.13 ± 0.04 | 0.02 ± 0.01 | 0.03 | 45.8 ± 2.1 | 47.2 ± 8.6 | 0.87 |

^{a}“Total” indicates B lymphocyte percentage of total cells in the indicated organ
^{b}B cell subsets identified as in FIG. 2-3

TABLE V
VH125Tg/V_{K}125^{SDNeo} B cell subset numbers (×10⁴ cells)

| | | Btk- | Btk- | t test p |
|---|---|---|---|---|
| Organ | B Cell Subset | Sufficient | Deficient | value |
| Bone Marrow | Total | 102.2 ± 4.9 | 132.2 ± 19.3 | 0.15 |
| Bone Marrow | Pro/Pre | 31.0 ± 4.3 | 49.1 ± 8.5 | 0.07 |
| Bone Marrow | Immature | 47.2 ± 3.8 | 66.4 ± 12.2 | 0.15 |
| Bone Marrow | Mature | 16.1 ± 3.5 | 9.0 ± 2.4 | 0.11 |
| | Recirculating | | | |
| | Total | 948.7 ± 114.6 | 393.0 ± 78.2 | 0.0011 |
| Spleen | | | | |
| Spleen | T1 | 35.5 ± 7.1 | 57.6 ± 13.4 | 0.15 |
| Spleen | T2 | 136.0 ± 16.1 | 125.0 ± 35.3 | 0.77 |
| Spleen | Follicular | 213.0 ± 42.5 | 18.2 ± 6.1 | <0.001 |
| Spleen | Pre-Marginal | 22.4 ± 7.2 | 9.2 ± 2.5 | 0.12 |
| | Zone | | | |
| Spleen | Marginal Zone | 516.6 ± 67.6 | 166.2 ± 32.4 | <0.001 |
| | Total | 17.4 ± 5.6 | 5.0 ± 1.0 | 0.03 |
| Pancreatic | | | | |
| Draining | | | | |
| Lymph Nodes | | | | |
| Pancreas | Total | 1.9 ± 0.8 | 0.2 ± 0.1 | 0.08 |

^{a}B cell subsets identified as in FIG. 2-3

TABLE VI
VH125Tg/V_{K}125^{SDNeo} non-insulin-binding and insulin-binding B cell subset
numbers (×10⁴ cells)

| | | Blk- | Btk- | | Btk- | Btk- | |
|---|---|---|---|---|---|---|---|
| | | Sufficient | Deficient | | Sufficient | Deficient | |
| | B Cell | Non-Insulin- | Non-Insulin- | t test | Insulin- | Insulin- | t teet |
| Organ | Subset | Binding | Binding | p value | Binding | Binding | p value |
| Bore | Immature | 6.9 ± 1.4 | 11.1 ± 2.4 | 0.16 | 39.5 ± 4.9 | 54.1 ± 11.9 | 0.27 |
| Marrow | | | | | | | |
| Bone | Maitre | 6.8 ± 1.7 | 4.8 ± 1.5 | 0.39 | 9.1 ± 1.9 | 4.0 ± 1.0 | 0.03 |
| Marrow | Recirculating | | | | | | |
| Spleen | Total | 420.0 ± 72.4 | 224.9 ± 52.4 | 0.047 | 528.7 ± 61.7 | 168.1 ± 40.1 | <0.001 |
| Spleen | T1 | 15.3 ± 3.7 | 17.1 ± 3.7 | 0.73 | 20.8 ± 3.7 | 43.7 ± 13.3 | 0.10 |
| Spleen | T2 | 88.4 ± 13.9 | 77.6 ± 25.7 | 0.71 | 54.1 ± 7.0 | 57.8 ± 17.5 | 0.84 |
| Spleen | Follicular | 90.8 ± 21.4 | 12.8 ± 4.6 | 0.004 | 128.2 ± 24.6 | 6.2 ± 2.1 | <0.001 |
| Spleen | Pre- | 8.4 ± 3.2 | 6.3 ± 1.8 | 0.58 | 13.9 ± 3.9 | 3.1 ± 0.7 | 0.02 |
| | Marginal | | | | | | |
| | Zone | | | | | | |
| Spleen | Marginal | 221.4 ± 40.1 | 118.5 ± 26.9 | 0.053 | 311.7 ± 39.2 | 57.2 ± 12.1 | <0.001 |
| | Zone | | | | | | |
| Pancreatic | Total | 10.0 ± 3.6 | 3.4 ± 0.7 | 0.06 | 7.2 ± 2.1 | 1.6 ± 0.3 | 0.007 |
| Draining | | | | | | | |
| Lymph | | | | | | | |
| Nodes | | | | | | | |
| Pancreas | Total | 1.0 ± 0.4 | 0.1 ± 0.1 | 0.08 | 0.9 ± 0.3 | 0.1 ± 0.1 | 0.08 |

^{a}B cell subsets identified as in FIG. 2-3

The fold change in B cell numbers was calculated for each developmental subset, differentiating between insulin-binding and non-insulin-binding B cells from the same mice, e.g. the average number of insulin-binding Btk-sufficient follicular B cells was divided by the average number of insulin-binding Btk-deficient follicular B cells (or the reverse for fold increase). The same was also calculated for non-insulin-binding B cells. Btk-deficiency preferentially reduced insulin-binding follicular (21-fold), pre-marginal zone (4-fold), and marginal zone (5-fold) B cell numbers, compared with the reduction in non-insulin-binding follicular (7-fold), pre-marginal zone (no change), and marginal zone (2-fold) B cell numbers (FIG. 5D). These data confirm that self-reactive anti-insulin B cells depend more heavily on BTK than non-autoreactive B cells, and that this differential dependency emerges in the follicular, pre-marginal zone and marginal zone subsets, rather than at the earlier transitional stages.

Btk-Deficient Insulin-Binding B Cells are Normally Positioned for Bone Marrow Exit in the Sinusoids.

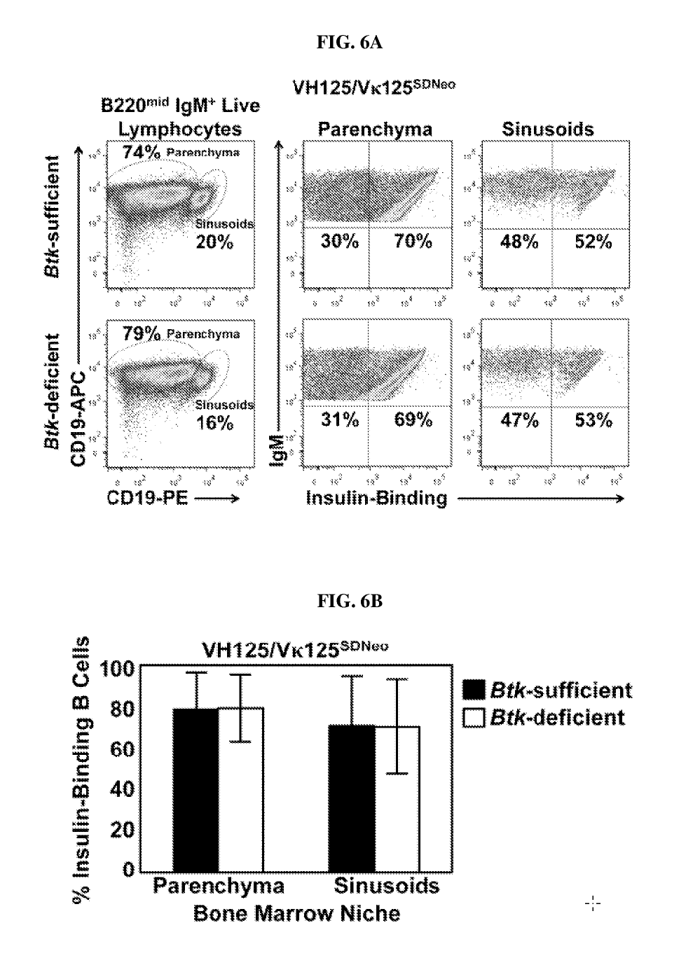

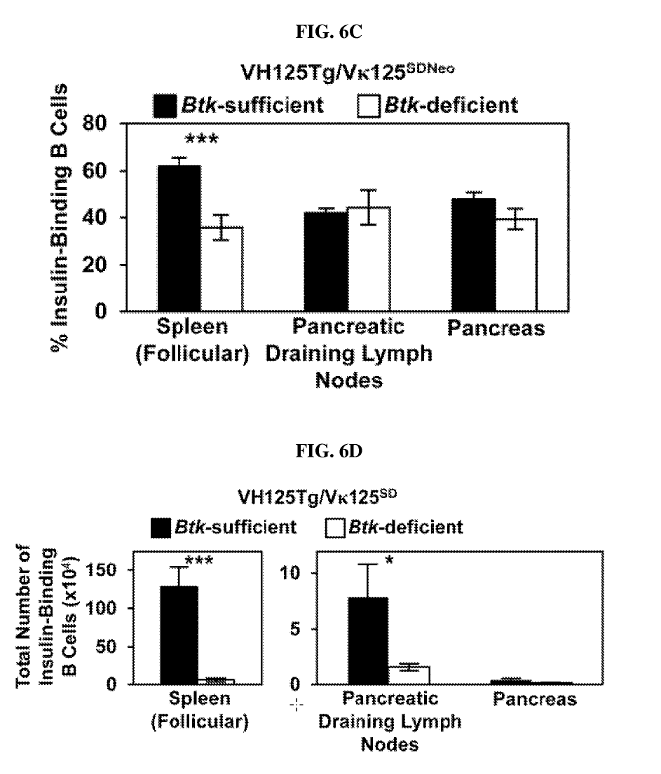

The VH125Tg/Vκ125^{SDNeo} model also allows comparison of factors governing migration of insulin-binding and non-insulin-binding B cells from bone marrow to spleen, and their level of dependency on BTK. As BTK has been shown to support chemotactic responses, we evaluated whether anti-insulin B cells had altered reliance on BTK compared with nonautoreactive B cells. Cell surface levels of CXCRS, which governs homing to secondary lymphoid follicles, was reduced in all BTK-deficient B cells, regardless of specificity (not shown). Functional analysis related to bone marrow egress by immature B cells was also performed. B lymphocytes in the bone marrow initially localize to the parenchyma, but migrate to the sinusoids prior to egress to the periphery. To investigate whether BTK supports this process in insulin-specific B cells, we labeled sinusoid B cells by injecting anti-CD19-PE intravenously, followed by euthanasia after 2 min. This preferentially labels B cells already in contact with blood flow in the sinusoids over those positioned away from the blood in the parenchyma. B cells were then harvested from bone marrow and stained with antibodies to delineate immature B cells, as well as anti-CD19-APC to help delineate the populations. Increased CD19-PE staining identifies B cells positioned in the sinusoids, compared with parenchymal B cells that stain with lower levels of CD19-PE. FIG. 6A shows immature gated (IgM⁺/B220^{mid}/CD23^{neg}) sinusoidal and parenchymal populations from Btk-sufficient and Btk-deficient V_{H}125Tg/Vκ125^{SDNeo}/NOD mice. The data show that Btk-deficiency does not affect the proportion of insulin-binding immature B cells (FIG. 6B) in the sinusoids, suggesting that BTK-mediated signaling does not contribute preferentially to their exit from the bone marrow.

Btk-Deficiency does not Preferentially Prevent Anti-Insulin B Cells from Reaching the Pancreatic Draining Lymph Nodes and Infiltrating the Pancreas.

Btk-deficiency reduces anti-insulin B cells at mature B cell stages in the spleen. To investigate whether Btk-deficiency further impacts the ability of anti-insulin B cells to home to the draining pancreatic lymph nodes and to infiltrate the pancreas in NOD mice, the average frequency of anti-insulin B cells present in these organs was compared among V_{H}125Tg/Vκ125^{SDNeo}/NOD Btk-sufficient and deficient mice. As shown in FIG. 6C-D, Btk-deficiency does not impair the ability of anti-insulin B cells to traffic to the pancreatic draining lymph nodes. Furthermore, anti-insulin B cells infiltrate the pancreas with similar frequency in the absence of BTK signaling (FIG. 6C). Thus, the significant reduction of mature Btk-deficient anti-insulin B cells in the spleen relative to non-autoreactive counterparts is not reflected at the sites of inflammation. Rather, these Btk-deficient autoreactive B cells are still able to respond to inflammatory signals and infiltrate the site of autoimmune attack.

BCR Internalization of Insulin is Btk-Independent.

To define the role of BTK in anti-insulin B cell function that promotes T1D, factors related to disease outcomes in the 125Tg/NOD model were analyzed. BCR internalization is critical for autoantigen processing and presentation to T cells. Loss of BTK reduces BCR internalization following anti-IgM stimulation. To identify whether BTK controls BCR internalization of a small, physiologic autoantigen by anergic B cells, BCR internalization of insulin was assessed in Btk-deficient or sufficient 125Tg/NOD mice. Surprisingly, the large majority of insulin was internalized with comparable kinetics in Btk-deficient and Btk-sufficient 125TgNOD mice within 10 min among all B cell subsets characterized (FIG. 7A). Surface IgM levels remained relatively constant during this time period in all subsets (FIG. 7A). Similar studies were performed using anti-IgMa. Consistent with previously published results, Btk-deficient follicular B cells showed diminished BCR internalization following stimulation with biotinylated anti-IgM^{a} (not shown). These data indicate that insulin internalization through the BCR is not altered by Btk-deficiency and suggest that the mechanism of internalization of a small, soluble, low affinity antigen may differ from that incurred by higher affinity crosslinking antigenic stimulation.

Btk-Deficient 125Tg B Cells Support Diabetes Development in NOD Mice.

Btk-deficiency protects against T1D in WT NOD mice. Disease is restored in that model when an anti-insulin BCR heavy chain transgene (V_{H}125Tg) is introduced. V_{H}125Tg/NOD/Btk-deficient mice have reduced, but measurable, numbers of anti-insulin B cells, as well as a large B cell population with a broad repertoire of non-insulin-specific B cells that may include other autoantigen specificities. By contrast, the 125Tg/NOD/Btk-deficient mice described in this report have very few B cells remaining, nearly all of them specific for insulin, providing the opportunity to determine directly whether anergic, anti-insulin B cells require BTK-mediated signaling to support development of T1D. The number of anti-insulin B cells was reduced in both Btk-deficient pancreatic draining lymph nodes (1.2±1.1 vs. 33.9±19.7×10⁴ cells, p<0.001) and pancreata (0.3±0.1 vs. 4.1±1.3, p=0.014), FIG. 7, Table II, commensurate with reductions seen in the spleen. As shown in FIG. 7C, diabetes onset is somewhat delayed in Btk-deficient 125Tg/NOD mice, but by age 30 weeks, the proportion with disease approaches that of their Btk-sufficient 125Tg/NOD littermates (60% vs. 69%, p=0.235). In contrast, despite having such low numbers of B cells, Btk-deficient 125Tg/NOD mice have significantly higher levels of disease than Btk-deficient non-transgenic NOD controls expressing endogenous BCR repertoires (12%, p<0.05). Since the mechanism of T1D promotion by 125Tg B cells is most likely antigen-presentation, these data imply that antigen-presenting function is intact in Btk-deficient 125Tg B cells.

### Example 2

Study Type: Interventional

Study Design:
Allocation: Randomized
Endpoint Classification: Safety/Efficacy Study
Intervention Model: Parallel Assignment
Masking: Double Blind (Subject, Caregiver, Investigator, Outcomes Assessor)
Primary Purpose: Treatment

Primary Outcome Measures:

Criteria are met for diabetes onset as defined by the American Diabetes Association (ADA) based on glucose testing or the presence of unequivocal hyperglycemia with acute metabolic decompensation.

Secondary Outcome Measures:

Effects on ibrutinib based on age, gender, race/ethnicity, weight, BMI, immunologic, genetic, demographic, and lifestyle factors.

Active Comparator: ibrutinib

Oral ibrutinib is given for once daily.

Placebo Comparator: Placebo sugar pill

The placebo sugar pill is given once daily.

The main study objective is to determine whether intervention with ibrutinib will prevent or delay the development of type 1 diabetes in individuals with anti-insulin B-cells. Secondary outcomes are to include analyses of C-peptide and other measures from Oral Glucose Tolerance Testing (OGTT), safety, tolerability, and other mechanistic outcomes will be assessed during the study.

Eligibility

Ages Eligible for Study: 8 Years to 45 Years

Genders Eligible for Study: Both

Criteria

Inclusion Criteria:

Between ages of 8-45 years

Have a relative with type 1 diabetes

If first degree relative must be 8-45 years old (brother, sister, parent, offspring)

If second degree relative must be between 8-20 years old (niece, nephew, aunt, uncle, grandchild, cousin)

Abnormal glucose tolerance by OGTT confirmed with 7 weeks of baseline visit [fasting blood glucose greater than 110 mg/dL or and less than 126 mg/dL OR 2 hour glucose greater or equal to 140 mg/dL and less than 200 mg/dL OR 30, 60, or 90 minute value on OGTT greater than or equal to 200 mg/dL]

Presence of at least two confirmed diabetes autoantibodies

Exclusion Criteria:

Type 1 diabetes previously diagnosed or detected at screening [fasting glucose greater or equal to 126 mg/dL or 2 hour glucose greater or equal to 200 mg/dL]

Abnormalities in blood counts, liver enzymes, INR,

Positive PPD test

Vaccination with live virus within 6 weeks of randomization

Evidence of acute infection based on laboratory testing or clinical evidence

Serological evidence of past current or past HIV, hepatitis B, or hepatitis C infection

Be currently pregnant or lactating

Prior treatment with study drug

### Example 3

An individual presents with impaired fasting glycemia and impaired glucose tolerance. The individual is determined to be prediabetic. The individual is administered ibrutinib and cyclosporine A. The individual does not develop Type 1 Diabetes.

What is claimed is:

1. A method for preventing or delaying the onset of type 1 diabetes in an individual in need thereof comprising administering to the individual in need thereof a composition comprising a therapeutically-effective amount of a compound of Formula A: 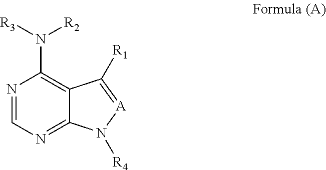
wherein
A is independently selected from N or CR₅;
R₁ is H, L₂-(substituted or unsubstituted alkyl), L₂-(substituted or unsubstituted cycloalkyl), L₂-(substituted or unsubstituted alkenyl), L₂-(substituted or unsubstituted cycloalkenyl), L₂-(substituted or unsubstituted heterocycle), L₂-(substituted or unsubstituted heteroaryl), or L₂-(substituted or unsubstituted aryl), where L₂ is a bond, O, S, —S(═O), —S(═O)₂, C(═O), -(substituted or unsubstituted C₁-C₆ alkyl), or -(substituted or unsubstituted C₂-C₆ alkenyl);
R₂ and R₃ are independently selected from H, lower alkyl and substituted lower alkyl;
R₄ is L₃-X-L₄-G, wherein,
L₃ is optional, and when present is a bond, optionally substituted or unsubstituted alkyl, optionally substituted or unsubstituted cycloalkyl, optionally substituted or unsubstituted alkenyl, optionally substituted or unsubstituted alkynyl;
X is optional, and when present is a bond, O, —C(═O), S, —S(═O), —S(═O)₂, —NH, —NR₉, —NHC(O), —C(O)NH, —NR₉C(O), —C(O)NR₉, —S(═O)₂NH, —NHS(═O)₂, —S(═O)₂NR₉—, —NR₉S(═O)₂, —OC(O)NH—, —NHC(O)O—, —OC(O)NR₉—, —NR₉C(O)O—, —CH═NO—, —ON═CH—, —NR₁₀C(O)NR₁₀—, heteroaryl, aryl, —NR₁₀C(═NR₁₁)NR₁₀—, —NR₁₀C(═NR₁₁)—, —C(═NR₁₁)NR₁₀—, —OC(═NR₁₁)—, or —C(═NR₁₁)O—;
L₄ is optional, and when present is a bond, substituted or unsubstituted alkyl, substituted or unsubstituted cycloalkyl, substituted or unsubstituted alkenyl, substituted or unsubstituted alkynyl, substituted or unsubstituted aryl, substituted or unsubstituted heteroaryl, substituted or unsubstituted heterocycle;
or L₃, X and L₄ taken together form a nitrogen containing heterocyclic ring;
G is 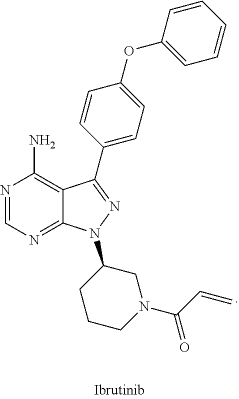
wherein,
R₆, R₇ and R₈ are independently selected from among H, lower alkyl or substituted lower alkyl, lower heteroalkyl or substituted lower heteroalkyl, substituted or unsubstituted lower cycloalkyl, and substituted or unsubstituted lower heterocycloalkyl;
R₅ is H, halogen, -L₆-(substituted or unsubstituted C₁-C₃ alkyl), -L₆-(substituted or unsubstituted C₂-C₄ alkenyl), -L₆-(substituted or unsubstituted heteroaryl), or -L₆-(substituted or unsubstituted aryl), wherein L₆ is a bond, O, S, —S(═O), S(═O)₂, NH, C(O), —NHC(O)O, —OC(O)NH, —NHC(O), or —C(O)NH;
each R₉ is independently selected from among H, substituted or unsubstituted lower alkyl, and substituted or unsubstituted lower cycloalkyl;
each R₁₀ is independently H, substituted or unsubstituted lower alkyl, or substituted or unsubstituted lower cycloalkyl; or
two R₁₀ groups can together form a 5-, 6-, 7-, or 8-membered heterocyclic ring; or
R₁₀ and R₁₁ can together form a 5-, 6-, 7-, or 8-membered heterocyclic ring; or
each R₁₁ is independently selected from H or alkyl; and
pharmaceutically active metabolites, pharmaceutically acceptable solvates,
pharmaceutically acceptable salts, or pharmaceutically acceptable prodrugs thereof.

2. The method of claim 1, wherein the compound of Formula A is (R)-1-(3-(4-amino-3-(4-phenoxyphenyl)-1H-pyrazolo[3,4-d]pyrimidin-1-yl)piperidin-1-yl)prop-2-en-1-one

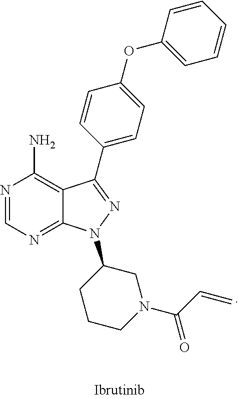 [SMILES: C=CC(=O)N1CCC[C@@H](n2nc(-c3ccc(Oc4ccccc4)cc3)c3c(N)ncnc32)C1]

3. The method of claim 1, wherein the method prevents the onset of Type 1 Diabetes or delays the onset of Type 1 Diabetes.

4. The method of claim 1, wherein the number of anti-insulin B cells in the individual is reduced.

5. The method of claim 1, wherein the individual presents with active autoimmunity to at least two of insulin, GAD65, IA-2, and Znt8.

6. The method of claim 1, wherein the individual presents with an elevated level of A1C.

7. The method of claim 1, wherein the individual presents with impaired fasting glycemia.

8. The method of claim 8, wherein the individual presents with fasting plasma glucose level from 5.6 mmol/L (100 mg/dL) to 6.9 mmol/L (125 mg/dL), or from 6.1 mmol/L (100 mg/dL) to 6.9 mmol/L (125 mg/dL).

9. The method of claim 1, wherein the individual presents with impaired glucose tolerance.

10. The method of claim 1, further comprising co-administering an additional therapeutic agent.

11. A method for preventing or delaying the onset of type 1 diabetes in an individual in need thereof comprising administering to the individual in need thereof a composition comprising a therapeutically-effective amount of (R)-1-(3-(4-amino-3-(4-phenoxyphenyl)-1H-pyrazolo[3,4-dlpyrimidin-1-yl]piperidin-1-yl)prop-2-en-1-one

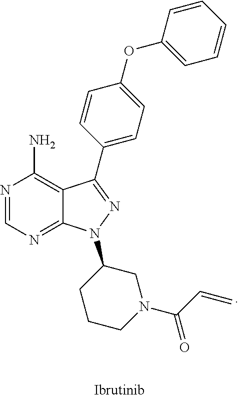 [SMILES: C=CC(=O)N1CCC[C@@H](n2nc(-c3ccc(Oc4ccccc4)cc3)c3c(N)ncnc32)C1]

12. A method for inhibiting the maturation of anti-insulin B cells in an individual in need thereof comprising administering to the individual in need thereof a composition comprising a therapeutically-effective amount of a compound of Formula A: 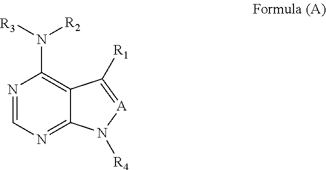
wherein
A is independently selected from N or CR₅;
R₁ is H, L₂-(substituted or unsubstituted alkyl), L₂-(substituted or unsubstituted cycloalkyl), L₂-(substituted or unsubstituted alkenyl), L₂-(substituted or unsubstituted cycloalkenyl), L₂-(substituted or unsubstituted heterocycle), L₂-(substituted or unsubstituted heteroaryl), or L₂-(substituted or unsubstituted aryl), where L₂ is a bond, O, S, —S(═O), —S(═O)₂, C(═O), -(substituted or unsubstituted C₁-C₆ alkyl), or -(substituted or unsubstituted C₂-C₆ alkenyl);
R₂ and R₃ are independently selected from H, lower alkyl and substituted lower alkyl;
R₄ is L₃-X-L₄-G, wherein,
L₃ is optional, and when present is a bond, optionally substituted or unsubstituted alkyl, optionally substituted or unsubstituted cycloalkyl, optionally substituted or unsubstituted alkenyl, optionally substituted or unsubstituted alkynyl;
X is optional, and when present is a bond, O, —C(═O), S, —S(═O), —S(═O)₂, —NH, —NR₉, —NHC(O), —C(O)NH, —NR₉C(O), —C(O)NR₉, —S(═O)₂NH, —NHS(═O)₂, —S(═O)₂NR₉—, —NR₉S(═O)₂, —OC(O)NH—, —NHC(O)O—, —OC(O)NR₉—, —NR₉C(O)O—, —CH═NO—, —ON═CH—, —NR₁₀C(O)NR₁₀—, heteroaryl, aryl, —NR₁₀C(═NR₁₁)NR₁₀—, —NR₁₀C(═NR₁₁)—, —C(═NR₁₁)NR₁₀—, —OC(═NR₁₁)—, or —C(═NR₁₁)O—;
L₄ is optional, and when present is a bond, substituted or unsubstituted alkyl, substituted or unsubstituted cycloalkyl, substituted or unsubstituted alkenyl, substituted or unsubstituted alkynyl, substituted or unsubstituted aryl, substituted or unsubstituted heteroaryl, substituted or unsubstituted heterocycle;
or L₃, X and L₄ taken together form a nitrogen containing heterocyclic ring;
G is 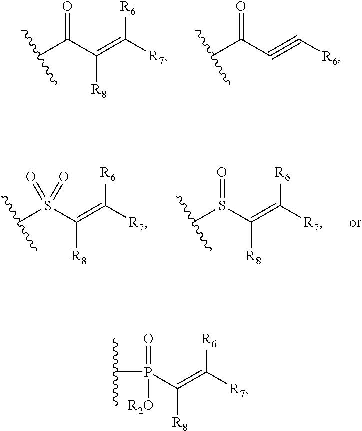
wherein,
R₆, R₇ and R₈ are independently selected from among H, lower alkyl or substituted lower alkyl, lower heteroalkyl or substituted lower heteroalkyl, substituted or unsubstituted lower cycloalkyl, and substituted or unsubstituted lower heterocycloalkyl;
R₅ is H, halogen, -L₆-(substituted or unsubstituted C₁-C₃ alkyl), -L₆-(substituted or unsubstituted C₂-C₄ alkenyl), -L₆-(substituted or unsubstituted heteroaryl), or -L₆-(substituted or unsubstituted aryl), wherein L₆ is a bond, O, S, —S(═O), S(═O)₂, NH, C(O), —NHC(O)O, —OC(O)NH, —NHC(O), or —C(O)NH;
each R₉ is independently selected from among H, substituted or unsubstituted lower alkyl, and substituted or unsubstituted lower cycloalkyl;
each R₁₀ is independently H, substituted or unsubstituted lower alkyl, or substituted or unsubstituted lower cycloalkyl; or
two R₁₀ groups can together form a 5-, 6-, 7-, or 8-membered heterocyclic ring; or
R₁₀ and R₁₁ can together form a 5-, 6-, 7-, or 8-membered heterocyclic ring; or
each R₁₁ is independently selected from H or alkyl; and
pharmaceutically active metabolites, pharmaceutically acceptable solvates,
pharmaceutically acceptable salts, or pharmaceutically acceptable prodrugs thereof.

13. The method of claim 12, wherein the compound of Formula A is (R)-1-(3-(4-amino-3-(4-phenoxyphenyl)-1H-pyrazolo[3,4-d]pyrimidin-1-yl)piperidin-1-yl)prop-2-en-1-one

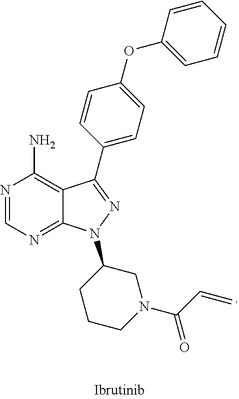 [SMILES: C=CC(=O)N1CCC[C@@H](n2nc(-c3ccc(Oc4ccccc4)cc3)c3c(N)ncnc32)C1]

14. The method of claim 12, wherein the number of anti-insulin B cells in the individual is reduced.

15. The method of claim 12, wherein the method prevents the onset of Type 1 Diabetes or delays the onset of Type 1 Diabetes.

16. The method of claim 12, wherein the individual presents with active autoimmunity to at least two of insulin, GAD65, IA-2, and Znt8.

17. The method of claim 12, wherein the individual presents with an elevated level of A1C.

18. The method of claim 12, wherein the individual presents with impaired fasting glycemia.

19. The method of claim 18, wherein the individual presents with fasting plasma glucose level from 5.6 mmol/L (100 mg/dL) to 6.9 mmol/L (125 mg/dL), or from 6.1 mmol/L (100 mg/dL) to 6.9 mmol/L (125 mg/dL).

20. The method of claim 12, wherein the individual presents with impaired glucose tolerance.
